(19) United States
(12) Patent Application Publication
(10) Pub. No.: US 2020/0237867 A1
(43) Pub. Date: Jul. 30, 2020

(54) METHODS AND DEVICES FOR PROMOTING NERVE GROWTH AND REGENERATION

(71) Applicant: Board of Regents, The University of Texas System, Austin, TX (US)

(72) Inventors: Mario I ROMERO-ORTEGA, Richardson, TX (US); Sanjay ANAND, Richardson, TX (US); Nesreen ALSMADI, Richardson, TX (US); Geetanjali S BENDALE, Richardson, TX (US)

(21) Appl. No.: 16/755,089

(22) PCT Filed: Oct. 12, 2018
(86) PCT No.: PCT/US2018/055556

§ 371 Date: Apr. 9, 2020

Related U.S. Application Data
(60) Provisional application No. 62/571,425, filed on Oct. 12, 2017.

(51) Int. Cl. A61K 38/18; A61K 38/17; A61L 27/38; A61B 17/11

(52) U.S. Cl. CPC A61K 38/185; A61K 38/1709; A61L 2430/32; A61B 17/11; A61L 27/3878

(57) ABSTRACT

In one aspect, methods of promoting asymmetric nerve growth and/or regeneration are described herein. In some embodiments, such a method comprises exposing a population of transected or severed nerves to a first molecular growth cue and to a second molecular growth cue. The population of transected nerves comprises one or more nerves of a first nerve type and one or more nerves of a second nerve type differing from the first nerve type. Additionally, the first molecular growth cue preferentially promotes growth of the first nerve type, as compared to the second nerve type. Similarly, the second molecular growth cue preferentially promotes growth of the second nerve type, as compared to the first nerve type. Moreover, the first molecular growth cue is spatially separated from the second molecular growth cue.

### CROSS REFERENCE TO RELATED APPLICATIONS

This application claims priority pursuant to 35 U.S.C. § 119 to U.S. Provisional Patent Application No. 62/571,425, filed on Oct. 12, 2017, which is hereby incorporated by reference in its entirety.

### STATEMENT REGARDING FEDERALLY SPONSORED RESEARCH

This invention was made with government support under grant N66001-11-1 and grant N66001-11-C-4168 awarded by the Defense Advanced Research Projects Agency (DARPA) Microsystems Technology Office; and under grant R21NS072955-01A1 awarded by the National Institutes of Health/National Institute of Neurological Disorders and Stroke (NIH/NINDS). The government has certain rights in the invention.

### FIELD

This invention relates to methods and devices for promoting nerve growth and nerve regeneration.

### BACKGROUND

Prosthetic devices have advanced from traditional mechanical hooks performing simple open/close tasks to anthropomorphic robotic hands capable of complex movements with up to 22 degrees of freedom and equipped with multiple sensors and embedded controllers for implementing automatic grasp and providing sensory feedback. Despite such progress, current prostheses are controlled through surface electromyography (EMG) signals and are operated by visual or surrogate sensory feedback which complicates the use of the robotic limbs and contributes to the eventual abandonment of these devices due to lack of embodiment. Decoding motor intent for robotic limb control, and conveying specific sensory modalities from the electronic skin to the user have been proposed as viable alternatives. However, realizing these goals has been a challenge. Various proposals for decoding motor intent have failed to provide the desired benefits and/or suffer from one or more disadvantages. For example, some approaches are highly invasive for the patient. In some approaches, cuff electrodes are used on an amputated nerve, and electrical impulses are used to convey sensation. However, patients implanted with these devices have perceived abnormal paresthesia including tingling and burning sensations believed to be related to indiscriminate depolarization of multiple sensory modalities axons, including pain and temperature C-fibers. Therefore, there exists a need for improved devices and methods for decoding and/or distinguishing between nerves/axons of varying types.

Another current challenge in the area of nerve growth is related to the repair of large nerve lesions. Despite the regenerative capacity of the adult peripheral nervous system and the routine repair of small nerve defects (<3 cm) using decellularized nerve grafts or nerve conduits, the surgical repair of critical lesions larger than 4 cm remains a significant difficulty, with limited expectations for recovery of function even with the use of autologous grafts. Some existing approaches to repairing large nerve defects suffer from one or more disadvantages. For example, some approaches fail to match the regenerative capacity offered by autologous grafts. A continued need thus exists for improved devices and methods for repairing large nerve lesions, and for functionally restoring damaged nerves.

### SUMMARY

In one aspect, methods of promoting asymmetric nerve growth are described herein. In some embodiments, such a method comprises exposing a population of transected nerves to a first molecular growth cue and to a second molecular growth cue. The population of transected nerves, in some cases, comprises one or more nerves of a first nerve type and one or more nerves of a second nerve type differing from the first nerve type. In some instances, the first molecular growth cue preferentially promotes growth of the first nerve type, as compared to the second nerve type, and the second molecular growth cue preferentially promotes growth of the second nerve type, as compared to the first nerve type. Additionally, in some cases, the first molecular growth cue is spatially separated from the second molecular growth cue.

In some embodiments, nerves of the first nerve type preferentially grow toward a first spatial region comprising the first molecular growth cue, and nerves of the second nerve type preferentially grow toward a second spatial region comprising the second molecular growth cue. In another embodiment, the first spatial region is defined by a first lumen and the second spatial region is defined by a second lumen differing from the first lumen. In one preferred embodiment, the nerves of the first nerve type are motor nerves, and the nerves of the second nerve type are sensory nerves. For example, in some cases, the population of transected nerves comprises peripheral nerves, axons from neurons in the central nervous system, or somatic nerves.

In some instances, the first molecular growth cue comprises an attractive molecular growth cue for the first nerve type, and the second molecular growth cue comprises an attractive molecular growth cue for the second nerve type. In other instances, the first molecular growth cue comprises an attractive molecular growth cue for the first nerve type, and the second molecular growth cue comprises a repulsive molecular growth cue for the first nerve type. In still further instances, the first molecular growth cue comprises a repulsive molecular growth cue for the second nerve type, and the second molecular growth cue comprises a repulsive molecular growth cue for the first nerve type. It is also possible for the first molecular growth cue to comprise an attractive molecular growth cue for the first nerve type, and the second molecular growth cue to comprise a repulsive molecular growth cue for the second nerve type.

The first molecular growth cue, in some embodiments, comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).

The second molecular growth cue, in some embodiments, comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).

In still another embodiment, the first molecular growth cue and/or the second molecular growth cue comprises a netrin, Slit protein, ephrin, semaphorin, cell adhesion molecule, or a combination of two or more of the foregoing. For example, the first molecular growth cue or the second molecular growth cue can comprise Semaphorin 3A. Alternatively, the first molecular growth cue comprises GDNF, and the second molecular growth cue comprises PTN.

In another embodiment, methods of promoting asymmetric nerve growth further comprises exposing the nerves of the first nerve type and/or the nerves of the second nerve type to a third molecular growth cue. The third molecular growth cue promotes myelination of the nerves of the first nerve type and/or the nerves of the second nerve type. In some cases, the third molecular growth cue stimulates Schwann cells. Moreover, in some instances, the third molecular growth cue is provided in microparticles. The microparticles can have an average diameter between 0.5 and 3500 μm, between 1000 and 3500 μm, or between 1500 and 3500 μm. In some embodiments, exposing the nerves of the first nerve type and/or the nerves of the second nerve type to a third molecular growth cue comprises using a sustained release profile for the third molecular growth cue, such as for at least 20 days, or for some other time period needed to maintain a physiologically relevant concentration of the growth factor for a time period sufficient for completion of the desired nerve growth.

In some embodiments, the third molecular growth cue comprises a neuregulin (NRG). For example, the third molecular growth cue, in some embodiments, comprises neuregulin 1. In some preferred embodiments, the third molecular growth cue comprises neuregulin 1 type III (NRG1-III).

In another aspect, devices for promoting asymmetric nerve growth are described herein. In some embodiments, such a device comprises a lumen having a proximal end and a distal end, and a matrix material disposed in the lumen. In some instances, the proximal end of the lumen comprises a proximal opening operable to receive nerve tissue, and the distal end of the lumen comprises a distal opening operable to receive nerve tissue. In some embodiments, the distal opening is capped. In other instances, the matrix material defines one or more first microchannels and one or more second microchannels extending from the proximal end of the lumen toward the distal end of the lumen, and the first microchannels differ from the second microchannels.

Devices for promoting asymmetric nerve growth, in some cases, comprise a first molecular growth cue disposed within the first microchannels and a second molecular growth cue disposed within the second microchannels. The first molecular growth cue differs from the second molecular growth cue. Moreover, the first molecular growth cue preferentially promotes growth of a first nerve type, whereas the second molecular growth cue preferentially promotes growth of a second nerve type, as compared to the first nerve type. In addition, in some embodiments, the distal end of the lumen is bifurcated into a first branch and a second branch. For example, the first microchannels, in some cases, are disposed in the first branch and the second microchannels are disposed in the second branch. Alternatively, in other instances, the distal end of the lumen is not bifurcated (e.g., the lumen is a single, straight, unbranched lumen), and the first microchannels are disposed in a first region of the lumen (e.g., on the left half of the lumen), and the second microchannels are disposed in a second region of the lumen (e.g., on the right half of the lumen).

The first molecular growth cue of devices described herein, in some embodiments, comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).

The second molecular growth cue of devices described herein, in some embodiments, comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).

In some preferred embodiments, the first molecular growth cue comprises GDNF, and the second molecular growth cue comprises PTN. In another preferred embodiment, the first molecular growth cue comprises GDNF, and the second molecular growth cue comprises BDNF or PTN and Sema3A. In some cases, as disclosed herein, specific combinations of neurotropic factors and pleitropic factors provide synergistic nerve growth. In such instances, the amount of desired nerve regeneration can be enhanced compared to the use of individual growth factors.

In addition, devices for promoting asymmetric nerve growth, in some cases, further comprise a third molecular growth cue disposed within the first microchannels and/or second microchannels. The third molecular growth cue, in some embodiments, promotes remyelination of the first nerve type and/or the second nerve type. In some instances, the third molecular growth cue comprises a neuregulin (NRG). In one preferred embodiment, the third molecular growth cue comprises neuregulin 1 type III (NRG1-III). The use of a third molecular growth cue specifically designated to promote remyelination, when used in combination with other growth cues as described herein, can be particularly effective for achieving functional repair of regenerating nerves.

### BRIEF DESCRIPTION OF THE FIGURES

FIG. 1A illustrates a schematic of Y-conduit containing microparticles with a neurotrophic factor in one compartment and BSA neutral control in the other. The transected mixed sciatic nerve is placed in the common arm of the Y-conduit and distal ends are capped with 1.5% agarose.

FIG. 1B illustrates a timeline of the experimental procedures following implantation.

FIG. 1C shows a PTN cumulative release curve over 30 day period and scanning electron microscope images of PTN-MP with size ranging from 0.3-1.5 μm (inset; Scale bar=3 μm).

FIG. 1D shows a comparison of average axonal length. Compared to control and BSA MP, the average axonal length for PTN and PTN MP was significantly higher (N=3/ group; One-way ANOVA; F (3, 263)=33.52; R²=0.2766; *** indicates P≤0.001).

FIG. 1E is a representative image of DRGs exposed to PTN

FIG. 1F is a representative image of DRGs exposed to PTN-MP.

FIG. 2 illustrates the diffusion of PLGA microparticles loaded with NTF and BSA over a 30 day time period. At Day 1 there is a burst release followed by a gradient formation in the subsequent days. The top compartment represents PTN-MP release and BSA-MP release in the bottom compartment. Arrow heads indicate the approximate position of nerve regeneration.

FIG. 3A is an image of a Y-split nerve regeneration using neurotrophic factor loaded microparticles (NTF-MP) with no distal targets.

FIG. 3B is bar graph comparing the regenerative nerve arm diameter in control and experimental arms. Data represented as mean±SEM; N=3-6 animals/group.

FIG. 4A illustrates the setup for measuring Compound Nerve Action Potential (CNAP). Bipolar hook electrodes provided stimulus pulses and the response recorded distally from the Y-split regenerated nerve fascicles.

FIG. 4B shows a graph illustrating that the regenerated split fascicles are electrically conductive. Representative recording showing the stimulus artifact followed by A, B, and C peak-responses.

FIG. 4C is a scatter plot of individual CNAP peaks with Conduction Velocity (CV) evoked by each treatment. The dotted lines indicate slow, medium, and fast responses from conducting fibers.

FIG. 4D is a scatter plot corresponding to individual area under the curve (AUC). Black bars represent the median evoked response per treatment. (N=3-6 animals/group; data represents evoked peaks).

FIG. 5A is a schematic of the longitudinal section of the ventral spinal cord showing fluorogold positive cells (white arrows).

FIG. 5B is a bar graph quantifying the number of fluorogold positive (FG+) cells in each of the split Y-nerve. GDNF had a significantly higher number of regenerated motor neurons compared to BSA.

FIG. 5C is a schematic of DRG soma size distribution and representative image of fluorogold positive cells of varying size.

FIG. 6 is a collection of electron microscopy images (EM) of the regenerated split Y-nerve fascicles showing normal axonal morphology with apparent large myelinated axons in the nerve-caps. Medium size axons in the GDNF, PTN, BSAs, whereas NGF shows higher density of unmyelinated axons. Large and unmyelinated axons are represented by * and (arrow), respectively. Scale bar=10 μm.

FIG. 7 is a bar graph of unmyelinated axon and myelinated axon count. Data represented as mean±SEM. n=number of sampled EM pictures per group. (*P≤0.05, ****P≤0.0001)

FIG. 8 is a collection of representative SEM images of dual neurotrophic factors for motor (tibial, muscle-cap, and BDNF+GDNF) and sensory (sural, skin-cap, PTN, and NFG) targets showing normal axonal morphological composition. Large and unmyelinated axons are represented by * and (arrow) respectively. Scale bar=10 μm.

FIG. 9A is a bar graph quantifying the number of unmyelinated axons.

FIG. 9B is a bar graph quantifying the number of myelinated axons.

FIG. 9C is a bar graph of the myelinated axon fiber diameter.

FIG. 9D is a collection of scatter plots of G-ratio as a function of myelinated axon diameter. Dotted lines represent distribution of small, medium, and large axons. Data represented at mean±SEM. n=number of sampled EM pictures per group. (*P≤0.05, ***P≤0.001, ****P≤0.0001).

FIG. 9E is a collection of scatter plots of G-ratio as a function of myelinated axon diameter. Dotted lines represent distribution of small, medium, and large axons. Data represented at mean±SEM. n=number of sampled EM pictures per group. (*P≤0.05, ***P≤0.001, ****P≤0.0001).

FIG. 10A is a bar graph of FG+motor neurons quantified from the ventral spinal cord.

FIG. 10B is a bar graph of FG+sensory neurons from the DRG.

FIG. 10C is a bar graph of sensory to motor neuron ratio of the FG+cells. Data represented as mean±SEM. (N=3-6 animals/group; *P≤0.05).

FIG. 11 is a schematic of the in vitro Y-template PDMS mold. The 6.0 mm holes contain NTF-MP mixed in collagen and the cell chamber (bottom hole) is where the DRG will be place. The canals serve as a pathway for the regenerating axons.

FIG. 12 is a schematic representation of Y-conduit containing attractants and repellent to increased efficacy in axon subtype enrichment.

FIG. 13A is a bright field image of the DRG axonal extension in the NGF (a1) and Sema3A (a2) chamber.

FIG. 13B is a bar graph of normalized axonal length.

FIG. 13C is a bar graph of change in axon turning from the NGF and Sema3A compartments. ** indicates significant difference between the chambers (P≤0.01). Data present at mean±SEM.

FIG. 14 is a bar graph of sensory/motor neuron ratio showing Sema3A was not effective in altering the ratio in the presence of dual choice molecular attractants.

FIG. 15 is a bar graph of percent distribution of myelinated axon diameter less than 1 BDNF+Sema3A show significant decrease compared to BDNF only arm. * indicates significant difference between the chambers (P≤0.05, Bonferroni). Data present at mean±SEM.

FIG. 16A is a bar graph of the release profile of PTN-MPs and SEM images of the MPs and confocal images of DRG axonal growth (β-tubulin labeled in red) demonstrating the bioactivity of PTN-MPs.

FIG. 16B is a photograph illustrating the BNI fabrication diagrams: (top) placement of metal rods in a perforated silicon conduit which is filled with agarose, (middle) after gelation the rods inserted in a chamber with Ws/collagen are pulled out, casting the microchannels and simultaneously filling the lumen, (bottom) producing a final implantable device.

FIG. 16C is a photograph of BNI showing 8 microchannels with the MP-collagen solution (insert).

FIG. 16D is a collection of images of the implanted BNI sutured to both ends of the injured common peroneal (CP) nerve. Tib=tibial nerve. Scale bars: B) 5 μm (SEM), 1 mm (DRG). D) 250 μm (channel), 10 μm (insert). E) 2 mm.

FIG. 17A is a collection of photographs of regenerated nerves 12 weeks after implantation. In contrast to the failure observed in collagen-filled conduits, BNIs with collagen, VEGF-MPs or PTN-MPs showed effective nerve repair. Scale bars: 0.5 cm

FIG. 17B is a collection of photographs of H&E histology revealing robust axonal regeneration in the BNI microchannels. Scale bars: 250 μm.

FIG. 17C is a collection of photographs of NFP immunocytochemistry confirming lack of axonal growth in the nerve conduit, and successful nerve regeneration in those with a BNI implant. Scale bars: 50 μm.

FIG. 17D is a collection of photographs showing double labeling of axons (β-tubulin, green) and myelin (P0, red) distal to the implant showed successful axonal regeneration and re-myelination particularly in the VEGF and PTN groups. Scale bar: 0.5 mm.

FIG. 17E is a bar graph quantifying axonal growth, which showed a significant increase in number of axons per channel in those with pleiotrophic support, and bar graph illustrating a trend for nerve regeneration distal to the implant. ***=p<0.001

FIG. 18A is a photograph of functional recovery in animals. Animals were unable to show digit abduction (black arrow) up to 6 weeks after injury repair.

FIG. 18B is a photograph of functional recovery in animals. By 7 weeks, animals implanted with VEGF or PTN Ws showed improvements in toe spreading (red arrows).

FIG. 18C is a line graph of toe spread index. Nine weeks after repair, those with BNI PTN showed significant improvement compared to those implanted with collagen filled conduits.

FIG. 18D is a bar graph of muscle mass. None of the groups showed a significant recovery in the total mass of the tibialis anterior muscle at this time. ***=p<0.001

FIG. 19A is a bar graph showing quantitative analysis of 50 axons per explant in triplicate revealed that PTN, alone or in combination, doubled the axonal length.

FIG. 19B is a bar graph of percent axonal length. Conversely, the combination of PTN with GDNF or NT-3 significantly increased axon density compared to control. ***=p<0.001, **=p<0.01, *=p<0.05.

FIG. 20 is a collection of photographs of regenerated nerves across a critical gap. Those with sustained release of GDNF showed larger axons but with limited myelination. In contrast, those with PTN and with PTN-GDNF showed large myelinated axons and clusters of unmyelinated axons similar to those observed in the cut-resuture controls.

FIG. 21 is a box and whisker plot of axon diameter distribution, which confirmed the synergistic effect of GDNF-PTN compared BSA and individual growth factor treatments, but not better than the cut-resuture control. Scale bar: 5 μm.

FIG. 22A is a line graph of action potentials. Compound action potentials were evoked in the cut-resuture controls, but failed in all animals implanted with BSA, GDNF or PTN BNIs. Only 1 of 5 rabbits repaired with PTN-GDNF showed a CMAP with an approximately 10% of the amplitude generated in the controls.

FIG. 22B is a scatter plot of muscle mass. The tibialis anterior muscle mass did not improved compared to cut-resuture controls.

FIG. 22C is a line graph of toe spread index. Evaluation of toe-spread index after 5 months post injury confirmed that animals implanted with PTN-GDNF BNIs were significantly better compared to those implanted with BSA. ***=p<0.001, *=p<0.05.

FIG. 23A is a scatter plot illustrating the release profile of the of NRG1-SMDF encapsulated PLGA microspheres.

FIG. 23B is a table of NRG1-SMDF encapsulated PLGA microspheres characteristics.

FIG. 24 is a collection of photographs of histological sections on the distal portion of the regenerated nerve comparing the effective re-myelination (dark blue structures inside the green circles) in the autograft and the NRG groups.

FIG. 25A is a line graph of evoked compound action potential thresholds across the 4 cm regenerated nerve.

FIG. 25B is a line graph of evoked compound action potential thresholds across the 4 cm regenerated nerve.

FIG. 25C is a line graph of evoked compound action potential thresholds across the 4 cm regenerated nerve.

FIG. 25D is a line graph of evoked compound action potential thresholds across the 4 cm regenerated nerve.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. Similarly, a stated range of “1 to 10” should be considered to include any and all subranges beginning with a minimum value of 1 or more and ending with a maximum value of 10 or less, e.g., 1 to 5, or 4 to 10, or 3 to 7, or 5 to 8.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

I. Methods and Devices for Asymmetric Nerve Growth

In one aspect, methods and devices for promoting asymmetric nerve growth and/or nerve regeneration are described herein. In some embodiments, such a method comprises exposing a population of transected or severed nerves to a first molecular growth cue and to a second molecular growth cue. The population of transected nerves comprises one or more nerves of a first nerve type and one or more nerves of a second nerve type differing from the first nerve type. Additionally, the first molecular growth cue preferentially promotes growth of the first nerve type, as compared to the second nerve type. Similarly, the second molecular growth cue preferentially promotes growth of the second nerve type, as compared to the first nerve type. Moreover, the first molecular growth cue is spatially separated from the second molecular growth cue. Further, nerves of the first nerve type can preferentially grow toward a first spatial region comprising the first molecular growth cue, and nerves of the second nerve type can preferentially grow toward a second spatial region comprising the second molecular growth cue. It is to be understood that the first spatial region and the second spatial region differ from each other. For example, in some implementations, the first spatial region is, or is defined by, a first lumen and the second spatial region is, or is defined by, a second lumen differing from the first lumen. Such an arrangement of spatial regions may be obtained, in some instances, by carrying out the method using a bifurcated or Y-shaped nerve growth conduit or device described hereinbelow. However, a desired arrangement of spatial regions may be obtained in other manners as well. For example, in some instances, both the first and second spatial regions are present in the same straight, unbranched lumen, and the first spatial region is defined by first microchannels within the lumen (e.g., disposed on the left half of the lumen), and the second spatial region is defined by second microchannels within the lumen (e.g., disposed on the right half of the lumen).

A method described herein may be used to selectively, preferentially, or asymmetrically grow and/or regenerate various nerve types. Generally, the nerves can be either somatic nerves or autonomic nerves. In some cases, a method described herein is particularly used for somatic nerves, not autonomic nerves. Transected nerves that can be grown and/or regenerated by a method described herein can be peripheral nerves or nerve tracts in the central nervous system. In some especially preferred embodiments, the nerves of the first nerve type are motor nerves, and the nerves of the second nerve type are sensory nerves.

Turning again to the molecular growth cues of a method described herein, it is to be understood that a “molecular growth cue” can comprise or consist of a small molecule, a polymeric or oligomeric species (including a naturally-occurring or artificial polypeptide, protein, or nucleic acid), or any other chemical species that is operable as a signal or factor for positive or negative nerve growth, when exposed to neurons, nerves, or axons. Moreover, a given “molecular growth cue” can comprise or consist of a single chemical species or a mixture or combination of a plurality of separate and distinct chemical species (e.g., a mixture of different small molecules).

Additionally, the first and/or second molecular growth cue of a method described herein can be or comprise an attractive molecular growth cue, where an “attractive” cue is understood to refer to a cue that promotes nerve growth, including in a direction toward a spatial region comprising the attractive cue, as opposed to inhibiting nerve growth or having no effect on nerve growth. Moreover, such an “attractive” cue can be attractive for either the first nerve type or the second nerve type. For example, in some cases, the first molecular growth cue comprises an attractive molecular growth cue for the first nerve type, and the second molecular growth cue comprises an attractive molecular growth cue for the second nerve type.

Further, in some cases, the attractive molecular growth cue for the first nerve type is not an attractive molecular growth cue for the second nerve type, or is a less attractive molecular growth cue for the second nerve type, as compared to the first nerve type. Additionally, it is also possible for the attractive molecular growth cue for the first nerve type to be a repulsive nerve growth cue for the second nerve type. Similarly, it is to be understood that the attractive molecular growth cue for the second nerve type can be a non-attractive molecular growth cue for the first nerve type or a less attractive molecular growth cue for the first nerve type, as compared to the second nerve type. Moreover, in some cases, the attractive molecular growth cue for the second nerve type is a repulsive nerve growth cue for the first nerve type.

In contrast to an “attractive” molecular growth cue, it is to be understood that a “repulsive” nerve growth cue inhibits or discourages growth of a given nerve, including in a direction toward a spatial region containing the repulsive nerve growth cue. In general, either the first molecular growth cue or the second molecular growth cue of a method described herein can be a repulsive growth cue, particularly for a nerve type that is not desired to be grown in the direction of the repulsive molecular growth cue, or that is desired to be grown in the direction of the repulsive molecular growth cue to a lesser degree than other nerve types. In some specific implementations described herein, the first molecular growth cue comprises an attractive molecular growth cue for the first nerve type, and the second molecular growth cue comprises a repulsive molecular growth cue for the first nerve type.

Non-limiting examples of “attractive” molecular growth cues that may be used as the first molecular growth cue or the second molecular growth cue include the following species: neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), persephin (PSPN), and combinations of two or more of the foregoing.

Non-limiting examples of “repulsive” molecular growth cues that may be used as the first molecular growth cue or the second molecular growth cue include the following species or classes of species: netrins, Slit (or Sli) proteins, ephrins, semaphorins, cell adhesion molecules (CAMs), and combinations of two or more of the foregoing. Netrins include secreted molecules that can act to attract or repel axons by binding to their receptors, DCC and UNC5. Slit proteins include secreted proteins that normally repel growth cones by engaging Robo (Roundabout) class receptors. Ephrins are cell surface molecules that activate Eph receptors on the surface of other cells. This interaction can be attractive or repulsive. In some cases, ephrins can also act as receptors by transducing a signal into the expressing cell, while Ephs act as the ligands. Signaling into both the Ephrin- and Eph-bearing cells is called “bi-directional signaling.” The many types of semaphorins primarily include axonal repellents. Additionally, semaphorins can activate complexes of cell-surface receptors called plexins and neuropilins. CAMs include integral membrane proteins mediating adhesion between growing axons and eliciting intracellular signalling within the growth cone. CAMs are a major class of proteins mediating correct axonal navigation of axons growing on axons (fasciculation). There are two CAM subgroups: IgSF-CAMs (belonging to the immunoglobulin superfamily) and cadherins (Ca-dependent CAMs).

In some preferred embodiments, the first molecular growth cue of a method described herein comprises GDNF, and the second molecular growth cue comprises PTN. Such a “pair” of molecular growth cues is especially preferred when the nerves of the first nerve type are motor nerves, and the nerves of the second nerve type are sensory nerves.

Additionally, it is further to be understood that a method described herein can be extended beyond distinguishing or differentiating only two nerve types. A method described herein may particularly include the use of more than two distinct molecular nerve growth cues. In general, “n” different molecular growth cues can be used, wherein the n different cues are spatially separated from one another. In this manner, n different nerve types (which may coexist in a “mixed” nerve bundle) can be selectively, preferentially, or asymmetrically grown in n different directions.

In some embodiments, a method of promoting asymmetric nerve growth and/or regeneration further comprises exposing the nerves of the first nerve type and/or the nerves of the second nerve type to a third molecular growth cue that is distinct from the first and second molecular growth cue. In some instances, a step comprises exposing the nerves to a third molecular growth cue after exposing the population of transected or severed nerves to the first molecular growth cue and to the second molecular growth cue. The third molecular growth cue can be disposed in the first spatial region or lumen and/or in the second spatial region or lumen. The third molecular growth cue, in some embodiments, promotes myelination of the nerves of the first nerve type and/or the nerves of the second nerve type. In some cases, the third molecular growth cue can recruit or stimulate myelin-forming or myelin-producing cells. For example, in some instances, the third molecular growth cue promotes differentiation of precursor myelin-forming cells. In other instances, the third molecular growth cue stimulates or activates a differentiated myelin-forming or myelin-producing cell, including glial cells such as a Schwann cell or an oligodendrocyte. Stimulating or activating a myelin-forming cell can include stimulating the cell to produce or generate a myelin sheath. In some instances stimulating can include inducing differentiation of a progenitor cell into a myelin-forming cell. In other instances, stimulating can include recruitment of a myelin forming cell to the first nerve type and/or the second nerve type. In still further instances, stimulating can include inducing production of myelin.

A third molecular growth cue in some embodiments comprises one or more myelination-promoting factors. For example, myelination-promoting factors can be growth factors or proteins known to comprise the myelin sheath, stimulate formation of a myelin sheath, and/or activate myelin-forming cells. Non-limiting examples of myelination-promoting factors include, sox 10, myelin protein zero (P0), krox-20, nab1, nab2, SREBP, NFATc4, YY1, Pou3f1/2, Brahma-associated factor (BAF), HDACs, myelin basic protein (MBP), myelin-associated glycoprotein, nectin-like protein 2, nectin-like protein 4, NDRG-1, proteolipid protein 1 (PLP1), peripheral myelin protein 22 (PMP22), maltose-binding protein, tetraspanins such as PMP-22, Par3, and neuregulin family proteins, including neuregulin 1, neuregulin 1 type III (NRG1-III) or sensory and motor neuron-derived factor (SMDF), laminin, laminin receptors, Gpr 126, Adam 22, Lgi4, BDNF, NT-3, epidermal growth factor (EGF), fibroblast growth factor (FGF), insulin-like growth factor 1 (IGF-1), insulin-like growth factor-2 (IGF-2), semaphorin 3A, semaphoring 3F, semaphorin 4F, and steroids such as thyroxine (T4).

In some embodiments, the third molecular growth cue of a method described herein comprises one or more extrinsic myelination-promoting factors. For example, an extrinsic myelination-promoting factor can comprise a secreted protein or signaling molecule derived from a non-myelin-forming cell, such as neuron, that acts on a myelin forming cell, such as a Schwann cell. Some non-limiting examples of extrinsic myelination-promoting factors include neuregulin 1 type III (NRG1-III) or sensory and motor neuron-derived factor (SMDF), laminin, Gpr 126, Adam 22, Lgi4, BDNF, NT-3, epidermal growth factor (EGF), fibroblast growth factor (FGF), insulin-like growth factor 1 (IGF-1), insulin-like growth factor-2 (IGF-2), semaphorin 3A, semaphorin 3F, semaphorin 4F, and steroids such as thyroxine (T4).

In another aspect, devices for promoting asymmetric nerve growth are described herein. In some cases, such a device comprises a lumen having a proximal end and a distal end, and a matrix material disposed in the lumen. The proximal end of the lumen comprises a proximal opening operable to receive nerve tissue (e.g., a proximal portion of a severed or transected nerve or nerve bundle). Additionally, the distal end of the lumen comprises a distal opening operable to receive nerve tissue (e.g., a distal portion of the severed or transected nerve or nerve bundle). Further, the matrix material defines one or more first microchannels extending from the proximal end of the lumen toward the distal end of the lumen. The matrix material also defines one or more second microchannels extending from the proximal end of the lumen toward the distal end of the lumen. The first microchannels differ from the second microchannels. Additionally, in some cases, the first microchannels are grouped or clustered in a first spatial region of the lumen, and the second microchannels are grouped or clustered in a second spatial region of the lumen, such that the first and second microchannels are spatially separated from one another, as opposed to being intermingled or intermixed on a single microchannel-by-microchannel basis. Further, in some instances, a first molecular growth cue is disposed within the first microchannels of the device, and a second molecular growth cue is disposed within the second microchannels of the device.

As described above for methods of promoting asymmetric nerve growth, the first molecular growth cue differs from the second molecular growth cue. In particular, in some cases, the first molecular growth cue preferentially promotes growth of a first nerve type, as compared to a second nerve type differing from the first nerve type, and the second molecular growth cue preferentially promotes growth of the second nerve type, as compared to the first nerve type. The nerve types and molecular growth cues can be any of the nerve types and molecular growth cues described herein for methods of promoting nerve growth and/or regeneration. For example, in some instances, the nerves of the first nerve type comprise motor nerves; the nerves of the second nerve type comprise sensory nerves; the first molecular growth cue comprises GDNF; and the second molecular growth cue comprises PTN.

Moreover, in some embodiments of a device described herein, the distal end of the lumen is bifurcated into a first branch and a second branch. Such a device can thus be a “Y-shaped” device. A device having such a structure, in some cases, can be used to receive a “mixed” nerve bundle at the proximal end and then at least partially “segregate” or “enrich” nerves of various types using differing molecular growth cues, such that two distinct nerve fascicles or “sub-bundles” emerge from the distal end of the tube, where at least one of the exiting fascicles is substantially enriched or depleted in a given nerve type, as compared to the original mixed nerve bundle. Such “enrichment” or “depletion” can comprise an enrichment or depletion of a given nerve type by an amount of at least 10%, at least 15%, at least 20%, at least 25%, at least 30%, at least 40%, or at least 50% compared to the original mixed nerve bundle, the percentage being based on using, as the denominator, a ratio or amount of the given nerve in the original mixed nerve bundle. In some cases, the enrichment or depletion of a given nerve type is 5-60%, 5-50%, 5-40%, 5-30%, 5-25%, 5-15%, 10-60%, 10-50%, 10-40%, 15-60%, 15-50%, 15-40%, 20-60%, or 20-50%. It is further to be understood that a method described herein that does not necessarily use a device described herein can nevertheless achieve the same segregation or enrichment of nerve types as described above for devices.

In addition, as described above for methods of promoting asymmetric nerve growth and/or regeneration, a device described herein can be used to segregate or differentiate more than two differing nerve types. In general, n different nerve types can be segregated or differentiated. In some such embodiments, a device described herein includes n spatially distinct and separated groups of microchannels, the groups of microchannels comprising n differing molecular growth cues. Moreover, in some cases, the n groups of microchannels are disposed in n distinct branches at the distal end of the device. In some implementations, for example, first microchannels are disposed in a first branch and second microchannels are disposed in a second branch.

Furthermore, as described above for methods of promoting asymmetric nerve growth and/or regeneration, a device described herein can comprise a third molecular growth cue. In some instances, the third molecular growth cue is disposed within the device. The third molecular growth cue can be disposed within the first microchannels and/or the second microchannels. The third molecular growth cue preferentially promotes myelination or re-myelination of the first nerve type, the second nerve type, and/or the “nth” nerve type. Thus, the third molecular growth cue differs from the first molecular growth cue and the second molecular growth cue. For example, in some instances, the third molecular growth cue comprises a myelination-promoting factor. A myelination-promoting factor of a device described herein includes any myelination-promoting factor described above.

In some embodiments, the third molecular growth cue of a device described herein comprises one or more extrinsic myelination-promoting factors. For example, an extrinsic myelination-promoting factor can comprise a secreted protein or signaling molecule that acts on a myelin-forming cell, such as a Schwann cell. Some non-limiting examples of extrinsic myelination-promoting factors include neuregulin 1 type III (NRG1-III) or sensory and motor neuron-derived factor (SMDF), laminin, Gpr 126, Adam 22, Lgi4, BDNF, NT-3, epidermal growth factor (EGF), fibroblast growth factor (FGF), insulin-like growth factor 1 (IGF-1), insulin-like growth factor-2 (IGF-2), semaphorin 3A, semaphorin 3F, semaphorin 4F, and steroids such as thyroxine (T4).

A device described herein can also have one or more features in addition to those described above. For example, in some cases, a device described herein further comprises a fluid, such as a saline solution, disposed in one or more microchannels of the device. Additionally, in some instances, one or more microparticles are disposed in the fluid, the microparticles comprising one or more molecular growth cues or other factors, including a myelination-promoting factor.

In some instances, the microparticles facilitate using or achieving a sustained-release profile of the one or more molecular growth cues or other factors. It should be understood that a sustained-release profile releases an amount of the one or more molecular growth cues from the microparticles, the amount being within a desired concentration range, for a desired period of time or time frame. The release of the one or more molecular growth cues from the microparticles is continuous, sustained, maintained or otherwise relatively constant over the course of the desired time frame. For example, in some cases the sustained-release profile comprises a continuous release of the one or more molecular growth cues for at least 4 hours, at least 8 hours, at least 12 hours, at least 24 hours, at least 2 days, at least 3 days, at least 5 days, at least 7 days, at least 10 days, at least 14 days, at least 15 days, at least 20 days, at least 25 days, or at least 30 days. In other cases, the sustained-release profile comprises a continuous release of the one or more molecular growth cues for 1-35 days, 1-30 days, 1-25 days, 1-20 days, 1-15 days, 1-14 days, 1-10 days, 1-7 days, 1-5 days, 1-3 days, 1-2 days, or 0.25-1 days, 10-180 days, 10-90 days, 10-60 days, 10-40 days, 10-30 days, 20-180 days, 20-90 days, 20-60 days, 20-40 days, 20-30 days, 30-180 days, 30-90 days, 30-60 days, or 40-180 days.

Furthermore, a sustained release profile continuously releases a desired amount. The desired amount is within a desired concentration range. That is, the continuous release of the one or more molecular growth cues is maintained within a concentration range, wherein the rate of release can be based on the total amount of the one or more molecular growth cues disposed within or disposed on the microparticles. Additionally, the rate of release can be based on the composition of the microparticle. It should be understood that a sustained-release profile continuously releases an amount of the one or more molecular growth cues that does not depart from the desired concentration range until the expiration of the desired time frame. Moreover, in some embodiments, a molecular growth cue can be provided in more than one concentration, wherein the two or more concentrations can be contained within the same microchannel or different microchannels. For example, in some cases, about 1 ng/ml to about 1000 ng/ml of the one or more molecular growth cues is continuously released from the microparticles during the desired time frame. In other cases about 50 ng/ml to 1000 ng/ml, 50 ng/ml to 500 ng/ml, 100 ng/ml to 500 ng/ml, 100 ng/ml to 300 ng/ml of the one or more molecular growth cues is continuously released from the microparticles during the desired time frame. It should be understood that the amount of the one or more molecular growth cues released from the microparticles can vary at different time points within the desired time frame, but the amount does not depart from the desired concentration range until the expiration of the desired time frame.

Turning now to some specific components of devices, devices described herein comprise a lumen or tube having a proximal end and a distal end, wherein the proximal end of the lumen comprises an opening operable to receive nerve tissue and the distal end of the lumen comprises an opening operable to receive nerve tissue. The lumen and the openings of the lumen can have any size, shape, and structure not inconsistent with the objectives of the present invention. In some embodiments, for instance, the lumen has a substantially cylindrical shape. Alternatively, in other instances, the lumen has a branched shape, including a branched shape formed from a plurality of substantially cylindrical branches. Further, in some cases, a lumen described herein (or portion thereof) has an inner diameter between about 100 μm and about 50 mm, between about 1 mm and about 10 mm, or between about 1 mm and about 5 mm. In some cases, the lumen has a diameter greater than about 50 mm or less than about 100 μm. An opening of a lumen described herein, in some embodiments, can have the same inner diameter as the lumen. Alternatively, in other instances, the opening can have a smaller size than the inner diameter of the lumen. Further, in some cases, a lumen described herein has a length between about 1 mm and about 200 mm, between about 5 mm and about 100 mm, between about 10 mm and about 30 mm, or between about 50 mm and about 150 mm.

Additionally, a lumen described herein can comprise or be formed from any material not inconsistent with the objectives of the present invention. In some embodiments, for instance, the lumen is formed from a polymeric material such as a polyurethane, a polyester, a polycarbonate, a polycaprolactone, a polylactic acid (PLA), a collagen, a polytetrafluoroethylene (PTFE), a polymethylmethacrylate (PMMass.), an ethylene-vinylacetate copolymer (EVA), a polydimethylsiloxane (PDMS), a polyether polyurethane, a polyethyleneterephthalate (PET), a polysulfone (PS), a polyethyleneoxide (PEO) or polyethylene glycol (PEG), a polyethylene oxide-polypropylene oxide copolymer (PEO-PPO), a polyolefin such as polyethylene (PE) or polypropylene (PP), or a combination of one or more of the foregoing. In some instances, the lumen comprises a segment of implantation or catheter tubing, such as Micro-Renathane implantation tubing. Other materials may also be used.

Devices described herein also comprise a matrix material disposed in the lumen of the device, the matrix material comprising or defining one microchannel or a plurality of microchannels. The matrix material of a device described herein can comprise any number of microchannels not inconsistent with the objectives of the present invention. In some cases, for example, a matrix material comprises between 1 and 10 microchannels or between 1 and 5 microchannels. In other implementations, a matrix material comprises or defines more than 10 microchannels. In addition, the microchannels can have any size not inconsistent with the objectives of the present invention. In some embodiments, for instance, the microchannels have an average diameter between about 100 μm and about 2000 μm, between about 100 μm and about 1000 μm, or between about 300 μm and about 800 μm. In some cases, the microchannels have an average diameter of less than about 100 μm or greater than about 2000 μm. Further, the microchannels can have a length up to about 100% of the length of the lumen of the device (or portion thereof, such as a branch). Moreover, the size and/or number of microchannels in a device described herein, in some cases, can be selected based on the size of a nerve or nerve bundle to be treated by the device, wherein a larger nerve or nerve bundle may require a larger number and/or a larger size of microchannels for effective treatment. For example, in some cases, a device for the treatment of a nerve or nerve bundle having a diameter of about 1.5 mm may comprise three microchannels having a diameter of about 600 μm each.

In addition, the matrix material of a device described herein can comprise or be formed from any material not inconsistent with the objectives of the present invention. In some cases, for instance, a matrix material comprises or is formed from a polymeric material. In some embodiments, a matrix material comprises or is formed from a hydrogel, such as, for example, a biodegradable hydrogel. A “biodegradable” material, for reference purposes herein, comprises a material that can decompose within a biological environment, and may provide a non-toxic decomposition product. In some embodiments, a biodegradable material described herein comprises one or more ester bonds. A matrix material described herein can also comprise or be formed from a non-biodegradable material, including a non-biodegradable polymeric material. In some instances, a matrix material described herein comprises an agarose gel. Any agarose gel not inconsistent with the objectives of the present invention may be used. In some cases, for example, a matrix material comprises an agarose gel comprising at least about 3 weight percent agarose, at least about 4 weight percent agarose, or at least about 5 weight percent agarose, based on the total weight of the agarose gel. In some embodiments, a matrix material comprises an agarose gel comprising between about 3 weight percent and about 10 weight percent agarose, between about 3 weight percent and about 8 weight percent agarose, or between about 3 weight percent and about 4 weight percent agarose, based on the total weight of the agarose gel. In other cases, a matrix material comprises an agarose gel comprising less than about 3 weight percent or less than about 2 weight percent agarose, based on the total weight of the agarose gel. In some instances, a matrix material comprises an agarose gel comprising between about 1 weight percent and about 2.5 weight percent agarose, based on the total weight of the agarose gel. Additional non-limiting examples of matrix materials suitable for use in some embodiments of devices described herein include polylactic-co-glycolic acid (PLGA), polylactic acid (PLA), polycaprolactone, polyurethane, polyester, polycarbonate, collagen, polytetrafluoroethylene (PTFE), polymethylmethacrylate (PMMass.), an ethylene-vinylacetate copolymer (EVA), a polydimethylsiloxane (PDMS), polyether-polyurethane, a polyethyleneterephthalate (PET), a polysulfone (PS), a polyethyleneoxide (PEO) or polyethylene glycol (PEG), a polyethylene oxide-polypropylene oxide copolymer (PEO-PPO), a polyolefin such as polyethylene (PE) or polypropylene (PP), or a combination of one or more of the foregoing. Other matrix materials can also be used, alone or in combination.

Devices described herein, in some embodiments, further comprise a fluid disposed in one or more microchannels of the device. Any fluid not inconsistent with the objectives of the present invention may be used. In some cases, the fluid comprises a saline solution such as a sterile solution of sodium chloride in water. Non-limiting examples of saline solutions suitable for use in some embodiments described herein include normal saline (about 0.90% w/v NaCl) and hypertonic saline (about 3-7% w/v NaCl). Other saline solutions may also be used.

In addition, in some cases, one or more microparticles are disposed in the fluid of a device described herein. Moreover, the microparticles can comprise one or more molecular growth cues disposed within the interior and/or on the exterior surface of the microparticles. A molecular growth cue can be present in a microparticle described herein in any amount not inconsistent with the objectives of the present invention. In some embodiments, for instance, a molecular growth cue is present in a microparticle in an amount between about 0.0001 and about 1 weight percent, based on the total weight of the microparticle.

Further, a microparticle can have any size and shape and be formed from any material not inconsistent with the objectives of the present invention. In some embodiments, for example, a microparticle is a spherical or substantially spherical microparticle having a diameter between about 0.5 μm and about 5000 μm or between about 1 μm and about 3500 μm. In other embodiments a microparticle has an average diameter of about 0.5 μm to about 4500 μm, about 1 μm to 4000 μm, about 1 μm to 5 μm, 1 μm to about 10 μm, about 1000 μm to about 4000 μm, or about 2000 μm to about 3500 μm. Microparticles having other sizes and shapes may also be used. In addition, in some cases, a microparticle is formed from a polymeric material, including any polymeric material described hereinabove for a lumen or matrix material. Alternatively, in other instances, a microparticle is formed from an inorganic material such as silicon dioxide and/or titanium dioxide. Other materials may also be used. The use of microparticles described herein, in some embodiments, can permit the time-delayed release or sustained-release profile of molecular growth cues into the microchannels of a device described herein.

Various components of devices have been described herein. It is to be understood that a device according to the present invention can comprise any combination of components and features not inconsistent with the objectives of the present invention. For example, in some cases, a device described herein comprises any lumen described herein in combination with any matrix material described herein and any molecular growth cues described herein.

Further, a device described herein can be made in any manner not inconsistent with the objectives of the present invention. In some instances, for example, a casting and/or negative extrusion process is used to form a lumen or matrix material comprising one or more microchannels or lumens. In other embodiments, a device described herein is formed by 3D printing.

II. Methods and Devices for Nerve Regeneration Using Pleiotrophic and Neurotrophic Factors

In yet another aspect, methods and devices for regenerating a transected nerve are described herein that are not necessarily asymmetric or do not necessarily include promoting asymmetric nerve growth as between differing nerve types. In some such embodiments, the method comprises exposing a transected nerve to a combination of a pleiotrophic growth factor and a neurotrophic growth factor. As described further hereinbelow, combining both of the foregoing types of factors can provide unexpectedly good nerve growth and/or regeneration, as compared to providing only one or more neurotrophic factors. In some preferred embodiments, the pleiotrophic growth factor comprises PTN, and the neurotrophic growth factor comprises GDNF.

As described above with reference to asymmetric nerve growth, it is to be understood that a “pleiotrophic growth factor” can include a single chemical species or a mixture or combination of differing chemical species, where each species is a pleiotrophic growth factor or where the overall mixture or combination has a pleiotrophic growth effect. Similarly, it is to be understood that a “neurotrophic growth factor” can include a single chemical species or a mixture or combination of differing chemical species, where each species is a neurotrophic growth factor or where the overall mixture or combination has a neurotrophic growth effect. Specific combinations of pleiotrophic and neurotrophic growth factors are further described in the detailed description hereinbelow.

In addition, a method of regenerating a transected nerve, in some embodiments, further comprises exposing the transected nerve to a myelination-promoting factor. In some instances, a step comprises exposing a transected nerve to a myelination promoting factor after exposing the transected nerve to the combination of a pleiotrophic growth factor and a neurotrophic growth factor. A myelination-promoting factor can include any myelination-promoting factor as described above. Exposing the transected nerve to a myelination-promoting factor can recruit, differentiate, stimulate, or activate any myelin forming cell, such as a Schwann cell or an oligodendrocyte, or a glial progenitor cell. In some embodiments, the myelination-promoting factor comprises one or more extrinsic myelination-promoting factors. For example, an extrinsic myelination-promoting factor can comprise a secreted protein or signaling molecule that acts on a myelin-forming cell, such as a Schwann cell. Some non-limiting examples of extrinsic myelination-promoting factors include neuregulin 1 type III (NRG1-III) or sensory and motor neuron-derived factor (SMDF), laminin, Gpr 126, Adam 22, Lgi4, BDNF, NT-3, epidermal growth factor (EGF), fibroblast growth factor (FGF), insulin-like growth factor 1 (IGF-1), insulin-like growth factor-2 (IGF-2), semaphorin 3A, semaphoring 3F, semaphorin 4F, and steroids such as thyroxine (T4).

In some embodiments, a method of regenerating a transected nerve further comprises functionally restoring the transected nerve. Functionally restoring a transected nerve, as understood by one of ordinary skill in the art, can be defined by measuring the electrochemical conduction of the nerve, i.e., by measuring the nerve's ability to elicit an action potential. In addition, function recovery can be defined by measuring the recovery of sensory modalities and/or voluntary movement of the denervated muscles. For example, the electrochemical conduction of the restored nerve can be compared to the electrochemical conduction of a non-resected nerve or other “control” nerve capable of electrochemical conduction. Additionally, various known tests for examining sensory and motor function can be used. In some instances the transected nerve is functionally restored by at least 10%, at least 15%, at least 20%, at least 30%, at least 40%, at least 50%, at least 60%, at least 70%, or at least 80% of the non-resected or control nerve.

In still another aspect, devices for regenerating a transected nerve are described herein, wherein the device may or may not promote asymmetric nerve regeneration in the manner described above. In some cases, such a device comprises a lumen having a proximal end and a distal end, and a matrix material disposed in the lumen. The proximal end of the lumen comprises a proximal opening operable to receive nerve tissue (e.g., a proximate portion of a severed or transected nerve or nerve bundle). Similarly, the distal end of the lumen comprises a distal opening operable to receive nerve tissue (e.g., a distal portion of a severed or transected nerve or nerve bundle). Additionally, the matrix material defines one or more microchannels extending from the proximal end of the lumen toward the distal end of the lumen. A combination of a pleiotrophic growth factor, a myelination-promoting factor, and a neurotrophic growth factor is disposed within the microchannels. The factors can be disposed in the microchannels in any manner not inconsistent with the objectives of the present disclosure, including in a manner described hereinabove for asymmetric nerve growth. For example, in some cases, the factors are encapsulated or disposed within biodegradable microspheres or microparticles located within the microchannels. Further, the lumen, matrix material, and microchannels can have any of the general compositional and/or structural features described hereinabove for devices for asymmetric nerve growth.

Moreover, it is to be understood that a method described herein can be carried out using a device described herein, or without using such a device.

III. Methods and Devices for Promoting Myelination and/or Re-Myelination of Nerves

In yet another aspect, methods and devices for promoting myelination of a nerve are described herein. Methods and devices described in the present section can, in some instances, comprise de novo myelination, wherein myelination occurs on a previously unmyelinated nerve, either arising from new nerve growth or a previously unmyelinated mature nerve. In other instances, methods and devices for promoting myelination of a nerve comprise restoring myelination or re-myelination, wherein a method of promoting myelination of a nerve is performed on a previously myelinated nerve having damaged myelin.

A method of promoting nerve myelination is described herein, which, in some embodiments, comprises exposing a population of unmyelinated nerves to a myelination-promoting factor. A population of unmyelinated nerves can include one or more nerves of varying types, including any nerve type described hereinabove. The unmyelinated nerve can be a healthy nerve, a diseased nerve, a transected nerve, an injured nerve, or a damaged nerve. For example, an unmyelinated nerve can comprise a nerve substantially free of myelin, a nerve having damaged or injured myelin, a nerve having a missing section of myelin, a nerve that was previously myelinated and is now lacking myelin, or a nerve having new growth in need of myelin at the location of the new growth, including new growth from axonal sprouting or axonal elongation. A damaged or injured nerve can comprise a nerve having damaged myelin from any demyelinating disease, including autoimmune disease, an infectious disease, or exposure to a chemical or toxic substance. Alternatively, a damaged or injured nerve can comprise a nerve having damaged myelin from a trauma-related injury, including blunt force, laceration, or amputation.

In some embodiments, a myelination-promoting factor comprises any myelination promoting factor, as described hereinabove. The myelination-promoting factor, in some embodiments, promotes myelination of the unmyelinated nerves. In some cases, the myelination-promoting factor can recruit or stimulate myelin-forming or myelin-producing cells. For example, in some instances, the myelination-promoting factor promotes differentiation of precursor myelin-forming cells. In other instances, the myelination-promoting factor stimulates or activates a myelin-forming or myelin-producing cell, including glial cells such as a Schwann cell or an oligodendrocyte. Stimulating or activating a myelin-forming cell, in some instances, comprises stimulating the myelin-forming cell to produce or generate a myelin sheath. In some instances, stimulating a myelin-forming cell comprises inducing differentiation of a progenitor cell into a myelin-forming cell. In other instances, stimulating a myelin-forming cell comprises recruiting a myelin forming cell to the unmyelinated nerves.

A myelination-promoting factor, in some embodiments, comprises one or more growth factors or proteins known to comprise the myelin sheath, stimulate formation of a myelin sheath, and/or activate myelin-forming cells. Non-limiting examples of myelination-promoting factors include, sox 10, myelin protein zero (P0), krox-20, nab1, nab2, SREBP, NFATc4, YY1, Pou3f1/2, Brahma-associated factor (BAF), HDACs, myelin basic protein (MBP), myelin-associated glycoprotein, nectin-like protein 2, nectin-like protein 4, NDRG-1, proteolipid protein 1 (PLP1), peripheral myelin protein 22 (PMP22), maltose-binding protein, tetraspanins such as PMP-22, Par3, neuregulin family proteins including neuregulin 1, neuregulin 1 type III (NRG1-III) or sensory and motor neuron-derived factor (SMDF), laminin, laminin receptors, Gpr 126, Adam 22, and Lgi4.

In some embodiments, the myelination-promoting factor of a method described herein comprises one or more extrinsic myelination-promoting factors. For example, an extrinsic myelination-promoting factor comprises a secreted protein or signaling molecule derived from a non-myelin-forming cell, such as a neuron, that acts on a myelin-forming cell, such as a Schwann cell. Some non-limiting examples of extrinsic myelination-promoting factors include neuregulin 1 type III (NRG1-III) or sensory and motor neuron-derived factor (SMDF), laminin, Gpr 126, Adam 22, Lgi4, BDNF, NT-3, epidermal growth factor (EGF), fibroblast growth factor (FGF), insulin-like growth factor 1 (IGF-1), insulin-like growth factor-2 (IGF-2), semaphorin 3A, semaphoring 3F, semaphorin 4F, and steroids such as thyroxine (T4).

In some embodiments, a method of promoting nerve myelination may comprise providing continuous exposure of the population of unmyelinated nerves to the myelination-promoting factor. Continuous exposure includes providing an amount of the myelination-promoting factor, the amount being within a desired concentration range, to the unmyelinated nerves over a desired period of time. For example, in some cases, the population of unmyelinated nerves are continuously exposed to about 1 ng/ml to about 1000 ng/ml of the myelination-promoting factor during the desired time frame. In other cases the population of unmyelinated nerves are continuously exposed to about 50 ng/ml to 1000 ng/ml, 50 ng/ml to 500 ng/ml, 100 ng/ml to 500 ng/ml, 100 ng/ml to 300 ng/ml of the myelination-promoting factor during the desired time frame. In another example, the population of unmyelinated nerves are continuously exposed to the myelination-promoting factor for a desired period of time of at least 4 hours, at least 8 hours, at least 12 hours, at least 24 hours, at least 2 days, at least 3 days, at least 5 days, at least 7 days, at least 10 days, at least 14 days, at least 15 days, at least 20 days, at least 25 days, or at least 30 days. In other cases, the desired period of time for continuous exposure comprises for 1-30 days, 1-25 days, 1-20 days, 1-15 days, 1-14 days, 1-10 days, 1-7 days, 1-5 days, 1-3 days, 1-2 days, or 0.25-1 day. It should be understood that the exposure amount of the myelination-promoting factor can vary at different time points within the desired time frame, but the measured amount does not depart from the desired concentration range until the expiration of the desired time frame.

In other embodiments, a method of promoting nerve myelination may share one or more features, properties, or characteristics of methods and devices described hereinabove. For example, exposing a population of unmyelinated nerves to a myelination-promoting factor, in some embodiments, comprises using one or more devices, as described hereinabove. For example, in some embodiments, the myelination-promoting factor is provided in a microparticle to facilitate using a sustained-release profile of the myelination-promoting factor. Moreover, it is to be understood that a method described herein can be carried out using a device described herein, or without using such a device.

In another aspect, a device for promoting myelination of a nerve is described herein. Such a device, in some embodiments, can share one or more features, properties, or characteristics of any device described hereinabove. Such a device, in some embodiments, comprises a lumen having a proximal end and a distal end and a matrix material disposed in the lumen. In some instances, the proximal end of the lumen comprises a proximal opening operable to receive nerve tissue, and the distal end of the lumen comprises a distal opening operable to receive nerve tissue. The matrix material, in other embodiments of the device, defines one or more microchannels extending from the proximal end of the lumen toward the distal end of the lumen, and a myelination-promoting factor is disposed within the microchannels. The myelination-promoting factor can be disposed in the microchannels in any manner not inconsistent with the objectives of the present disclosure, including in a manner described hereinabove for asymmetric nerve growth or nerve regeneration. For example, in some cases, the myelination-promoting factor is encapsulated or disposed within biodegradable microspheres or microparticles located within the microchannels. Further, the lumen, matrix material, and microchannels can have any of the general compositional and/or structural features described hereinabove for devices for asymmetric nerve growth or nerve regeneration.

Some specific examples of such methods and devices will now be described in more detail.

### EXAMPLE 1

### Asymmetric Nerve Growth

### A. Introduction

Neural interfaces are designed to decode motor intent and evoke sensory precepts in amputees. In peripheral interfaces, decoding movement intent is challenging because motor axons are only a small fraction compared to sensory fibers and are heterogeneously mixed particularly at proximal levels. Herein, the differential potency of NGF, glial cell line-derived neurotrophic factor (GDNF), brain-derived neurotrophic factor (BDNF), pleiotrophin (PTenn.), and NT-3 is evaluated for asymmetrically guiding the regeneration of sensory and motor neurons. In the absence of distal target organs, molecular guidance cues can result in electrically conductive Y-nerves with normal microanatomy. Compared to Y-tube compartments with bovine serum albumin (BSA), GDNF and NGF increased the motor and sensory axon content, respectively. In addition, the sensory to motor ratio was significantly increased by PTN (12.7:1) when compared to a BDNF+GDNF choice. The differential content of motor and sensory axons modulated by selective guidance cues provides a strategy to better define axon types in peripheral nerve interfaces, as described herein by the present inventors.

Prosthetic devices have advanced from traditional mechanical hooks performing simple open/close tasks to anthropomorphic robotic hands capable of complex movements with up to 22 degrees of freedom and equipped with multiple sensors and embedded controllers for implementing automatic grasp and providing sensory feedback^{1,2}. Despite such progress, current prostheses are controlled through surface electromyography (EMG) signals and are operated by visual or surrogate sensory feedback which complicates the use of the robotic limbs and contributes to the eventual abandonment of these devices due to lack of embodiment³. Decoding motor intent for robotic limb control, and conveying specific sensory modalities from the electronic skin to the user have been proposed as viable alternatives. Cortical interfaces that are tailored towards individuals with spinal cord injury have shown great promise in achieving volitional control of a prosthetic limb and eliciting sensory precepts through microstimulation of the sensory cortex⁴⁻⁶. For upper limb amputees, peripheral nerve stimulation offers a less invasive alternative to cortical interfaces and a direct access to functional motor and sensory pathways in the residual limb⁷.

Several electrode configurations have been developed to interface with peripheral nerves, including extraneural cuffs, intrafascicular electrode arrays, and regenerative based electrodes^{8,9}. The external cuff and intrafascicular electrodes have been used successfully to elicit sensory feedback as well as recording motor intent in amputees. Typical precepts elicited include digit flexion, constant pressure, natural tapping, and vibration. Variations in stimulus parameters such as pulse width, amplitude, and frequency modulate the percept type and quality¹⁰⁻¹³. However, patients also perceived abnormal paresthesia including tingling and burning sensations^{10,12,14}, which has been related to indiscriminate electrical depolarization of multiple sensory modalities, including pain and temperature C-fibers, by the different electrodes¹⁵. This is believed to be due to the fact that axons of similar biophysical characteristics such as the large myelinated proprioceptive and mechanoreceptive fibers or medium myelinated slow and rapidly adapting mechanoreceptors have overlapping depolarizing thresholds¹⁶. Furthermore, recording selectively from motor axons from somatic nerves is challenging due to the small number compared to sensory axons and their heterogeneous distribution¹⁷.

The ability of transected nerves to regrow through multi-electrode arrays forming regenerative interfaces was demonstrated more than 20 years ago¹⁸. Using this approach, such nerves can be directed to grow through a 18-electrode array placed in the lumen of a nerve conduit^{19,20}. This regenerative multi-electrode interface (REMI) records single units as early as 7 days from both motor and sensory sub-modality axons from animals in which the interfaced nerves are allowed to reinnervate their original target in the skin and muscle²¹. There is an important role of neurotrophic factors (NTFs) from Schwann cells, muscle, and skin targets, both during early development and post-injury in the adult peripheral nerve system (PNS). Herein is described the use of such guidance cues in regenerative neural interfaces to influence the selective interaction of motor and sensory subtype-axons with distinct electrodes.

Motor axons follow chemoattractant gradients of glial cell line-derived neurotrophic factor (GDNF), through tyrosine kinase Ret and GFRα2 receptors²⁵. They also integrate membrane bound signals like Celsr3 cadherin in their path toward muscle targets^{26,27}. Conversely, pain and temperature related sensory fibers are attracted by gradients of nerve growth factor (NGF), mechanoceptive neurons are influenced by brain-derived neurotrophic factor (BDNF), and neurotrophin-3 (NT-3) is critical for reinnervation of proprioceptive neurons to muscle spindles²⁸⁻³². Furthermore, adding NGF to injured nerves can selectively guide the regeneration of TrkA nociceptive neurons^{33,34}, while NT-3 guides TrkC+ proprioceptors³⁵⁻³⁷, and BDNF stimulates TrkB-mechanoceptive fibers³⁸. Pleiotrophin (PTenn.), another neurotrophic factor, has also been shown to be expressed in ventral roots after injury and promotes spinal motor neuron regeneration³⁹. Indeed, in the adult PNS, exogenous NGF effectively guides the regeneration of nociceptive axons into the dorsal spinal cord after dorsal rhizotomy³³ and doubles the ratio of sensory to motor (S/M) axons innervating the NGF-expressing branch in the femoral nerve bifurcation injury model³⁴.

Herein, the present inventors describe the effect of single and combined NTFs in selectively guiding sensory and motor neurons into separate and closed compartments of a Y-shaped conduit after nerve transection in vivo. First, the effect of individual NTFs was evaluated against that of BSA, a neutral cue, in the other compartment of a Y-shaped conduit. Second, an in vivo assay was used to evaluate the pathway favored by regenerating axons when presented with competing choices in the form of multiple NTFs loaded in opposite compartments. The results demonstrate that GDNF had a stronger effect than PTN, NT3, BDNF, NGF to attract motor axons compared to BSA. Furthermore, the sensory-to-motor (S/M) ratio of regenerated axons was significantly increased in the PTN loaded side of the Y-conduit compared to the BDNF+GDNF compartment

### B. Material and Methods

### 1. Microencapsulation of Neurotrophic Factors (NTFs)

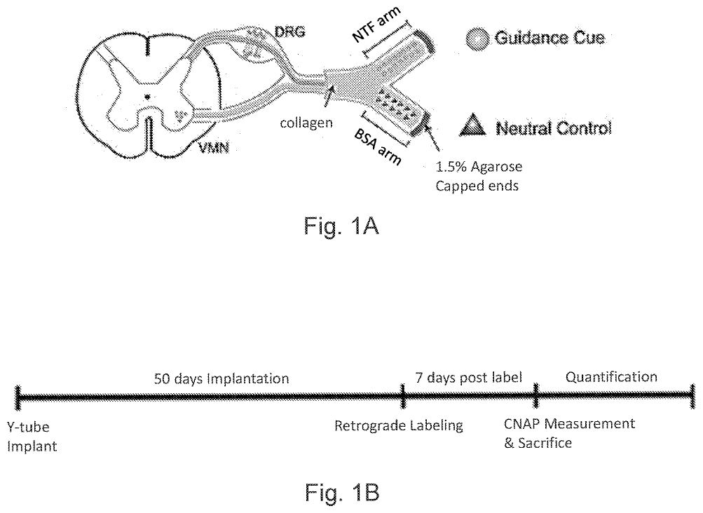

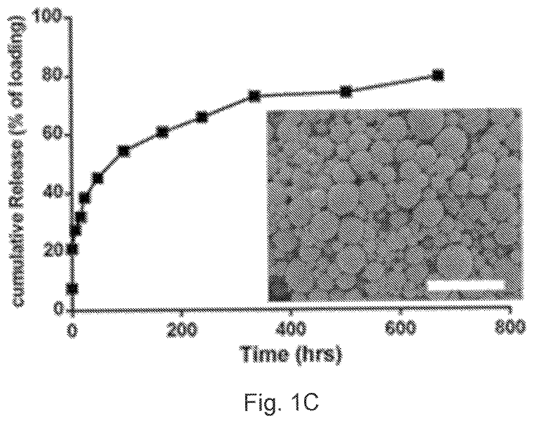

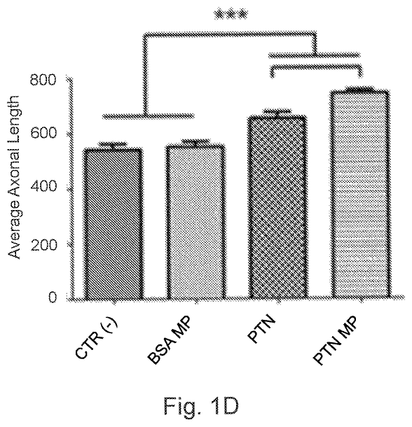

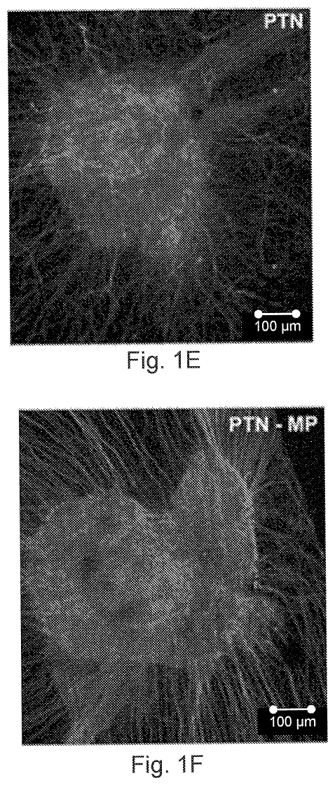

Biodegradable microparticles (MPs) were made with poly(DL-lactic-co-glycolic acid (PLGA) using double emulsion as reported previously⁴¹. Briefly, PLGA 50:50 (Lakeshore Biomaterial, St. Louis, Mo.) was dissolved in dichloromethane (DCM) 200 mg/ml (Sigma-Aldrich, St. Louis, Mo.), and mixed with aqueous solutions of human recombinant NGF (7S, 13.5 kD; Invitrogen), BDNF (27 kD), NT-3 (13.6 kD), GDNF (15 kD), or PTN (15.4 kD) (20 μg/ml; Prepotech Inc, N.J.) or BSA (20 μg/ml; Sigma-Aldrich, St. Louis, Mo.). The solutions were added to polyvinyl alcohol (20 mg/ml) and emulsified. The resulting MPs were freeze-dried for 48 hours and stored at −20° C. Loading efficacy was calculated at 67±5% from release studies. The particles were evaluated by scanning electron microscope (SEM, Hitachi S-3000 N), and their size distribution was estimated at 800 nm wavelength using a Zeta Potential Analyzer. Released NGF and PTN into phosphate buffer saline solution (PBS) from the MPs were evaluated at 37° C. in a shaker incubator at hourly intervals, daily for a week, and weekly after that for 4 weeks, and quantified by ELISA (PTN; TSZ ELISA, HU9951) (FIG. 1B). BSA release was quantified using the BCA assay (Thermo Scientific, Rockford, Ill.) and read at 562 nm.

### 2. DRG Bioactivity Assay

Neonatal (P0-P4) mice (CD1) were used to obtain dorsal root ganglia (DRGs) and collected in Leibovitz's L-15 Medium (Sigma-Aldrich, St. Louis, Mo.). The DRGs were cleaned of connective tissue and placed in poly-D-lysine (PDL) coated glass-bottom wells suspended in 10 μl of atelomeric chicken collagen (85% type I, 15% type II; Millipore; Temecula, Calif.). The explants were incubated at 37° C. with 5% CO₂ for 15 minutes to allow gelation before adding 200 μl of Neurobasal A media (Sigma-Aldrich, St. Louis, Mo.) supplemented with 2% B27, 0.5% penicillin/streptomycin, and 0.75% L-glutamine. NTF MPs were compared to recombinant proteins at previously reported physiological concentrations: NGF, BDNF and PTN were tested at a 100 ng/ml, GDNF at 50 ng/ml and NT-3 at 5 ng/ml. The NTFs and NTF-MPs were added 24 hours after plating the DRGs. Control DRG explants were incubated in BSA-MPs. After 5 days in culture, the DRGs were fixed for 15 minutes in 4% PFA, rinsed and stored at 4° C.

### 3. DRG In Vitro Immunocytochemistry

Fixed DRGs were permeabilized in 0.5% PBS-Triton X100 for 5 minutes. Non-specific staining was blocked with 4% normal donkey serum for 1 hour, followed by incubation with a mouse anti-β tubulin III antibody (1:400; Sigma-Aldrich, St. Louis, Mo.) overnight at 4° C. After rinsing, the tissue was incubated for 1 hour with a Cy2-conjugated donkey anti mouse antibody (1:400; Sigma-Aldrich, St. Louis, Mo.). The stained tissue was imaged on a Zeiss confocal microscope (Zeiss Axioplan 2 LSM 510 META). Axonal growth was estimated from 3 DRG samples per treatment. Z-stacks were imaged at 20× magnification (20 images each at 15.4 ␣m slice thickness) and individual axons were traced using ImageJ software measuring from the edge of the DRGs to the axon terminals (FIG. 1D-F).

### 4. Y-Tube Conduits

Poly(ester urethane) was synthesized from polycaprolactone, hexamethylene diisocyanate, and putrescine⁴². The poly(ester urethane) Y-shaped tubes were made using Y-shaped molds made with dental wax (Polysciences Inc., Warrington, Pa.). The molds were dip coated 20-30 times to achieve a wall thickness of approximate 0.25 mm in 5% polyurethane/hexafluoro isopropanol solution. The coated tubes were dried overnight at room temperature, and the dental wax was removed by immersion in hexane. The Y-tube then was dried in air at room temperature. The Y-conduit common arm measured 5 mm and each of the two compartments measured 5-7 mm from the bifurcation point, with 1.5 mm ID and total length of 10 -12 mm.

Y-tube conduits were disinfected with 70% ethanol followed by UV light irradiation. Collagen type VIII (EMD Millipore, Billerica, Mass.) was then used to fill the lumen of the Y-conduit. The common arm was filled with collagen using a 28-gauge insulin syringe up to the bifurcation and allowed to polymerize at 37° C. for 10 minutes. The “Y” compartments were then filled with 10 μL of NTF-MP or BSA-MP, mixed with collagen, and polymerized at 37° C. for 10 minutes before implantation.

### 5. Modeling Release of Neurotrophic Factors in Y-Shaped Conduits

The amount of NTF-MPs needed was estimated to provide sustained release for 30 days in each compartment of the Y-conduit by modeling the protein release and diffusion of the NTF MP as previously described⁴³. Briefly, finite element analysis (COMSOL, Inc.) was used to model the NTF concentration and diffusion in the collagen-filled lumen. One compartment of the Y-conduit was modeled for PTN release and another for BSA, according to the parameters specified in Table 1.

TABLE 1
Dimension and diffusivity values for protein
release from PLGA microparticles in collagen.

| Parameter | Value | | Description |
|---|---|---|---|
| D_{PTN} | 7.6 × 10⁻¹² m2/s ⁴³ | | Diffusivity of PTN in 0.1% |
| | | | collagen |
| D_{BSA} | 2.2 × 10^{-11 44} | | Diffusivity of BSA in 0.1% |
| | | | collagen |
| ID | 1.5 | mm | Channel internal diameter |
| L | 5 | mm | Channel length |
| v_{microchannel} | 10 | μl | Volume within conduit |
| | | | compartment |
| d_{microparticle} | 2 | μm | Diameter of PLGA micro- |
| | | | particles |
| Tₜₒₜₐₗ | 1.34 | ng | Total amount of releasable |
| | | | PTN in the compartment |

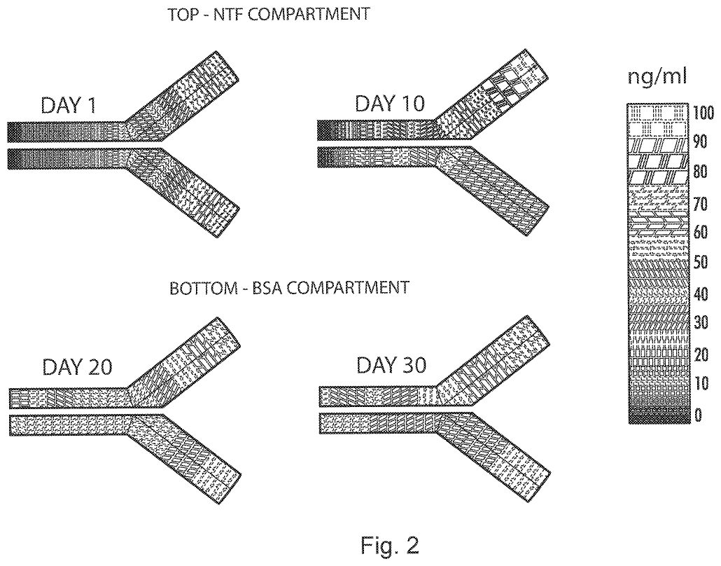

The model considers 2 μm diameter PLGA-MP, and assumes no degradation of collagen, and isotropic protein diffusion. Using this model, separate chemotactic gradients could be established in both compartments of the Y-conduits in the first 10 days (FIG. 2).

### 6. Animal Implantation

Two separate cohorts of adult female Lewis rats were included in this study. In the first cohort, (n=42), the effect of single molecular attractants in six experimental and one control group were evaluated (n=6 each): Mixed-nerve-cap (+control), and Y-conduits with NTF in one compartment (NGF, BDNF, NT-3, GDNF, and PTN, and BSA in the opposite side (Table 2). The nerve-cap was a 2 mm distal segment of the transected sciatic nerve ligated at one end and sutured to arms of the Y-conduit with the open end facing the lumen. The nerve-cap secretes multiple growth-factors and will have a non-specific effect on axonal regeneration. The sciatic nerve was used as the non-injured control to determine the baseline number of retrograde traced motor and sensory neurons. The second cohort of 60 rats was used to assess the dual molecular guidance in a Y-choice assay in two control and three experimental groups (n=12 each): 1) Tibial (motor related)−sural (sensory) nerve-cap targets (positive control), 2) Muscle (motor-related−Skin (sensory) tissue-cap targets (positive control), 3) BDNF+GDNF−BSA, 4) BDNF+GDNF−PTN, and 5) BDNF+GDNF−NGF.

TABLE 2
Experimental groups for single and combination
neurotrophic factors in a Y-choice assay.

| | Compart- | Compart- | Number of | |
|---|---|---|---|---|
| Cohort | ment 1 | ment 2 | implants | Objective |
| 1 | BDNF | BSA | 6 | Effect of individual |
| 1 | GDNF | BSA | 6 | Neurotrophic factors |
| 1 | NT-3 | BSA | 6 | against neutral |
| 1 | PTN | BSA | 6 | guidance cue BSA |
| 1 | NGF | BSA | 6 | |
| 1 | Nerve-cap | Nerve-cap | 6 | |
| 2 | BDNF + GDNF | BSA | 12 | Effect of |
| 2 | BDNF + GDNF | PTN | 12 | combinatorial |
| 2 | BDNF + GDNF | NGF | 12 | Neurotrophic |
| 2 | Tibial | Sural | 12 | factors in a |
| 2 | Muscle-cap | Skin-cap | 12 | Y-choice assay |

The animals were anesthetized using isoflurane (2-2.5%) in 100% oxygen prior to surgical procedure. Then the sciatic nerve was exposed by muscle-sparing incision between the semitendinosus and the bicep femoris muscles and transected before the trifurcation as described in reference⁴⁵. The distal portion of the nerve was removed to avoid trophic effect from the distal nerves and/or end targets. The sciatic nerve was then secured into the proximal arm of the Y-conduits using 9.0 nylon sutures. The distal ends of the Y-tube were capped by adding 1.5% agarose, placed under the muscle and closed using 4.0 silk suture. The skin was then closed using staples and topical antibiotic ointment was applied. All animals received antibiotic (cephazolin; 5 mg/kg, IM) and pain control (sustained release Buprenorphine; 0.1 mg/kg, SC) treatment post-surgery. All animal procedures were approved by the Institutional Animal Care and Use Committees (IACUC) of the University of Texas at Arlington and The University of Texas at Dallas, in accordance to the NIH Guide for the Care and Use of laboratory Animals.

### 7. Retrograde Labeling of Motor and Sensory Neurons and Quantification

Retrograde labeling from the distal end of the fascicles that regenerated into each of the Y-conduit compartments was used to trace the axon sub-types to its origin in the spinal cord and DRG. Briefly, the Y-tube was re-exposed 45 days-post implantation. With the Y-tube in place, sterile Vaseline was used to make a reservoir at the distal end of the Y-tube arm and injected at the bifurcation site to prevent leakage of the retrograde label into the common arm. Thereafter, 5 of 4% fluorogold (FG, Fluorochrome LLC, Denver, Colo.) was added to one arm and 5 μL of 10% fluororuby (FR, Fluorochrome LLC, Denver, Colo.) to the other arm of the exposed nerve for 1 hour. The Y-tube was then placed back under the muscle and closed using resorbable sutures and the skin stapled. The skin surface was coated with antibiotic ointment and the animal was given antibiotics (cephazolin; 5 mg/kg, IM) and pain control (sustained release Buprenorphine; 0.1 mg/kg, SC) treatment post-surgery. The neuronal cell bodies of the regenerated axons were visualized in the ventral spinal cord and DRG. FG tracing labeled 953±30.2 motor and 6914±603.9 sensory axons in the non-injured sciatic control nerves, with an estimated tracing efficiency of 60% for VMNs and 66% for somatic DRG neurons from a total 1600 VMN and 10,500 DRG neurons respectively^{46,47.} In contrast, FR labeling showed inefficient uptake compared to FG and therefore was omitted from the study.

### 8. Compound Nerve Action Potential (CNAP) Measurements

Seven days following the retrograde labeling procedure, the implant site was surgically reopened while the animal was anesthetized. The sciatic nerve and its regenerated arms were exposed, carefully freed from surrounding connective tissue, and a Parafilm® tape was placed underneath the nerves to ensure electrical isolation. The sciatic nerve was gently placed on a bipolar hook microelectrode (FHC Inc., Bowdoin, Me.) close to the pelvic foramen for stimulation. A second pair of hook electrodes was used to record evoked CNAP responses distally from the individual regenerated arms. Mineral oil covered the contact between the electrodes and the nerve. The sciatic nerve was stimulated with 30 μs wide biphasic pulses 2 Hz frequency for about 2 min duration using a A-M Systems (Sequim, Wash.) optically isolated instrument (model 2100). The CNAP from each regenerated arm of the Y-conduit was recorded in response to supra maximal stimulation amplitude, i.e. three times the threshold to activate onset-response, in a bandwidth of 3-5000 Hz and a 20×gain preamplifier using Omniplex Data Acquisition System (Plexon Inc., Dallas, Tex.). For offline time-synchronization, a copy of the stimulation output was directly fed into the data acquisition system. The recorded data was analyzed offline using MATLAB. A composite CNAP response was generated from 200-300 stimulation pulses using the Stimuli-triggered Averaging (STA) process. Briefly, signals were extracted in time-windows triggered 50 ms post and 15 ms prior to stimulus, overlapped and averaged to form a STA-CNAP waveform. Evoked peak responses were defined as an increase in amplitude larger than 10% from the baseline noise level which ranged from −0.003 to +0.003 mV. The peak latency was calculated by measuring duration between the positive phase of the stimulation pulse to the maximum amplitude of the individual peaks. The corresponding conduction velocities were obtained by dividing the distance (range of 15-25 mm) between the stimulating and recording electrodes over peak latencies. The area under curve for each peak was calculated from onset of the peak to the trough using the “trapz” inbuilt MATLAB's mathematical function, which approximates the integral using the trapezoidal method.

### 9. Animal Perfusion and Tissue Preparation

Animals were sacrificed by overdose injection of sodium pentobarbital (120 mg/kg, IP) and transcardially perfused with 4% paraformaldehyde (PFA). The regenerated Y-nerve were harvested and post fixed in 4% PFA/2.5% glutaraldehyde in 0.1 M Cacodylate buffer for electron microscopy (EM). The spinal cord and the L4 and L5 Dorsal root ganglion were isolated, post fixed and cryoprotected in 30% sucrose prior to OCT embedding for cryosectioning. Sagittal sections were obtained at 20 μm thickness and DRG cross sections at 10 μm thickness. The sections were mounted onto glass slides serially and stored at −20C until used.

### 10. Quantification of Retrograde Labeled Motor and Sensory Neurons

All slides were rinsed in PBS to remove the OCT and treated for lipofuscin reduction by incubating the tissue in 750 μM cupric sulfate/50 mM ammonium acetate for 40 minutes as reported⁴⁸. The tissue was then rinsed in PBS and cover-slipped using immuno-mount (Fisher Scientific, Waltham, Mass.). The FG+ and FR+ cells in the spinal cord and DRG were identified with a Zeiss fluorescence microscope using a wide band ultraviolet (UV) excitation filter. To prevent double counting, only positive retrograde labeled cells with a distinct nucleolus were included. Positively labeled DRG were also counted and the area was analyzed using the analyze-particle option of Image J, processing software. The DRG soma area was categorized into small (<300 μm²), medium (300-700 μm²), and large (<700 μm²). Labeled cell quantification was corrected by accounting for section thickness and split nuclei count using methods described⁴⁹.

### 11. Electron Microscopy Morphometry Analysis

A subset of groups (nerve-cap control, GNDF, NGF, PTN, BDNF+GDNF and respective BSA compartments) were processed for EM (n=3 animals per group; 3 random areas were imaged; 100-300 axons per area) to evaluate the axon type composition and myelination. The fixed tissue was embedded in resin and sectioned at 1 μm thickness using an ultra-microtome. The thin sections were stained with toluidine blue and photographed. Osmium stained sections were visualized using a JEOL LEM 1200 EX II microscope. Three fields of view per section with an area of 1520 μm² were imaged (4000× magnification) and analyzed for the number of unmyelinated and myelinated axons, fiber (axon+myelin) diameter, axon diameter, and g-ratio using Image J software.

### 12. Statistical Analysis

The groups were compared using one-way ANOVA and Bonferroni's ad-hoc multiple comparison test between the compartments of the Y-conduits using Prism 6 software (GraphPad Software Inc.). A p<0.05 was considered statistically significant. The data is presented as the mean±standard error of the mean.

### C. Results

### 1. Functional Splitting of Transected Peripheral Nerves

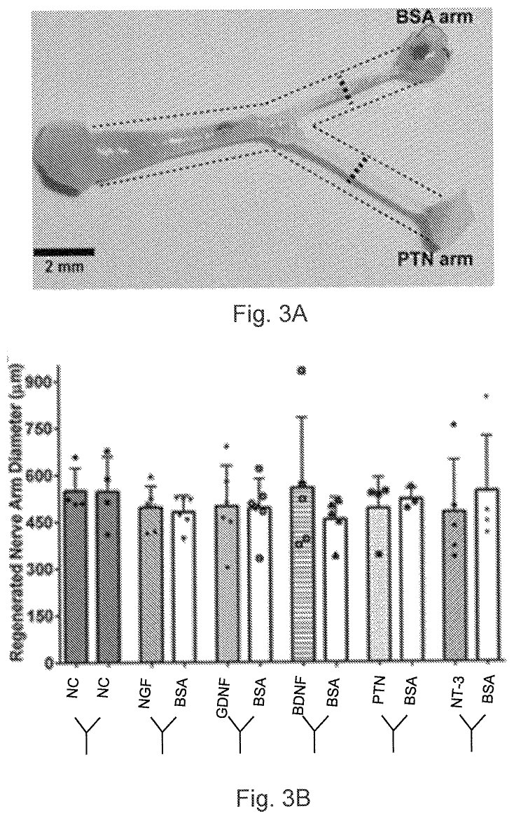

Gross evaluation of the regenerated nerves into the Y-shaped conduits 45 days after implantation confirmed that the injured nerves extended along the 5 mm common arm, and bifurcated into two similar size branches (4-5 mm long; FIG. 3A). The overall nerve growth into the two compartments was comparable in size (550 μm OD), filled 73.8±19.3% of the cross-sectional area (FIG. 3B), and elongated up to the agarose-capped end. Histological analysis confirmed the formation of perineurium and endoneurium in all groups. Qualitative evaluation of axonal composition in Toluidine-stained sections showed similar axonal growth among all groups. A cross-section of a nerve regenerated towards PTN (FIG. 3) or BSA in the same Y-conduit is was evaluated (FIG. 3).

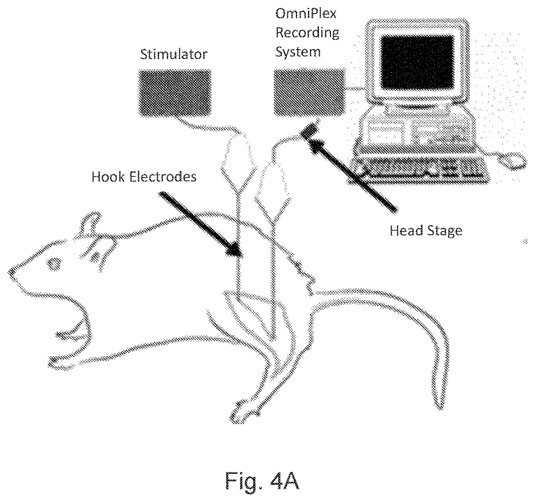

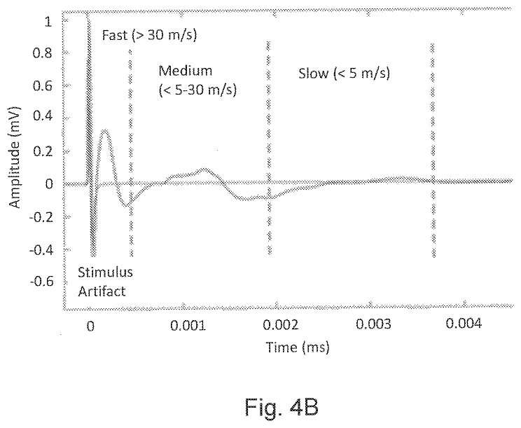

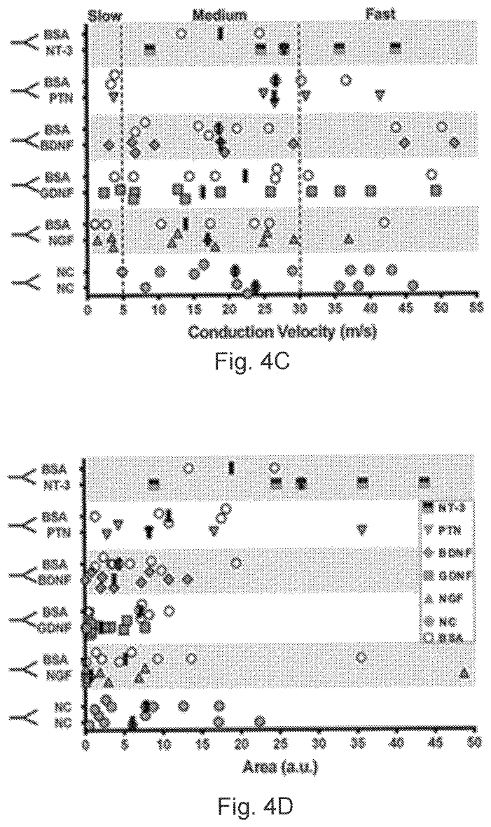

Ninety-seven percent of the split fascicles showed an evoked compound action potential, and only 3% had limited axonal growth and no CNAP response. Conduction velocity (CV) was calculated from the latencies of the peaks based on the distance from the stimulating and recording bipolar electrodes (FIG. 4A). Using 3× threshold potentials (1.5-3.0 V; FIG. 4B), slow (≤5 m/s), medium (5-30 m/s), and fast (>30 m/s) conducting CNAPs were evoked (FIG. 4C). The median responses were medium to fast in Y-tubes with GDNF (16.4 m/s), PTN (26.6 m/s), NT-3 (27.9 m/s), BDNF (18.9 m/s), and NGF (17.0 m/s), compared to nerve-caps (22.3 m/s) and averaged BSA (20.1 m/s). Quantification of the median peak areas showed small (NGF; 0.7 a.u.), medium (NT-3; 2.3, GDNF; 2.2, BDNF; 3.8 a.u.) and large (PTN; 6.0) peaks compared to nerve-caps (6.9 a.u.) and BSA (4.4 a.u.), demonstrating that the split fascicles are electrically conductive and providing some indication of the differential effect of the growth factors.

2. GDNF Increased the Regeneration of Both Motor and Sensory Neurons, while NGF and PTN Differentially Influence Sensory-Axon Regeneration.

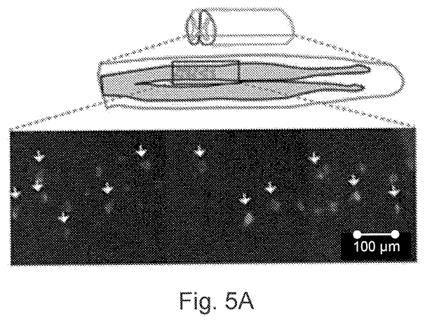

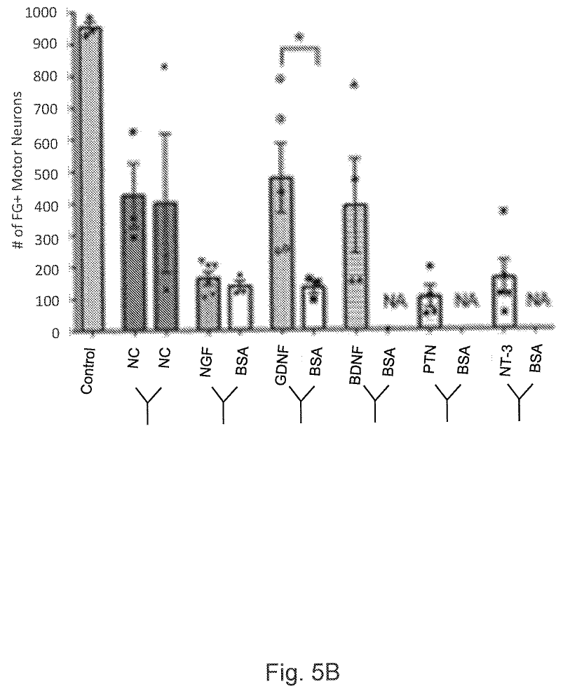

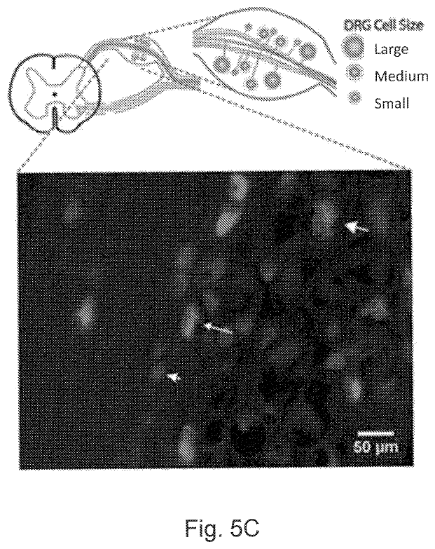

The number of ventral motor neurons that regenerated into the separated Y-conduit compartments in non-injured controls was approximately 1000, i.e. 62.5% of the expected 1600 total population (FIGS. 5A & B). Injured nerves attached to Y-conduits with nerve-caps as targets showed an even distribution of VMNs into both compartments (424±176.4 and 400.3±377.8). Conversely, when neurotrophic factors were used as distal targets, the different guidance cues significantly influenced the number of motor neurons innervating the two compartments (ANOVA: P≤0.05; F (6, 19)=8.824; R²=0.74). PTN (101.3±34.38), NGF (162.5±20.5), and NT-3 (160.0±54.5) only attracted approximately 30% of the motor neurons. In sharp contrast, those growing into BDNF (388.5±295.8), and GDNF (476.6±242.6) compartments, were comparable to the nerve-caps. GDNF specifically attracted significantly more motor neurons than BSA in the other compartment of the same conduit (133.7±33.9; P≤0.05).

Quantification of the traced sensory neurons in the DRG of non-injured sciatic nerve controls showed 6914±603.9 FG+cells. This number was reduced and highly variable in the compartments of Y-conduits with nerve-cap targets (1883±1042 and 1658±1353; FIG. 5C). Both GDNF (1819±1987) and BDNF (1540±1176) attracted similar number of DRG sensory neurons compared to nerve-cap. These numbers were reduced in those growing towards NGF (929±485.7), PTN (753.5±456.9), and NT-3 (306.8±362.9). To determine the effect of neurotrophic factors on sensory subtype regeneration, the DRG neurons were also evaluated based on perikaryal area (FIG. 5). However, no statistically significant difference was observed between the small, medium, and large DRG neurons attracted by each of the neurotrophic factors loaded compartment.

The sensory-to-motor ratio for each type of treatment was estimated to be 7.3±0.8 in non-injured sciatic nerves, which decreased slightly in Y-shaped conduits with nerve-cap controls (5.5±4.0 and 4.4±1.2) presumably due to the decreased sensory neuron regeneration following injury (FIG. 5)⁵⁰. The GDNF-compartment had the lowest sensory-to-motor ratio (3.1±2.1) while the PTN compartment had the highest (9.2±5.8) amongst all neurotrophic factor than BSA treatments and nerve-cap controls, but did not reach statistical significance. The sensory-to-motor ratios of compartments with NGF and GDNF, when compared to their respective BSA compartments, showed no significant effect. Together, these results suggest that specific neurotrophic factors can influence the axonal composition of split fascicles using a Y-conduit.

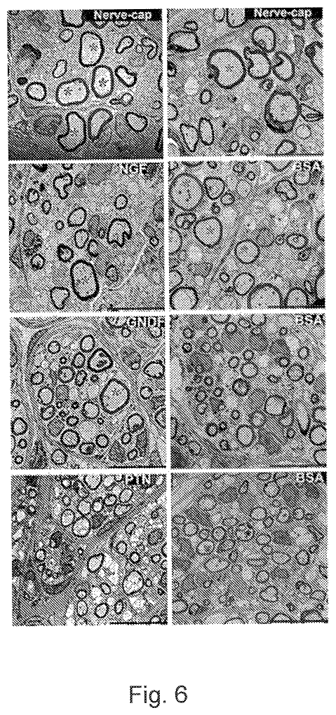

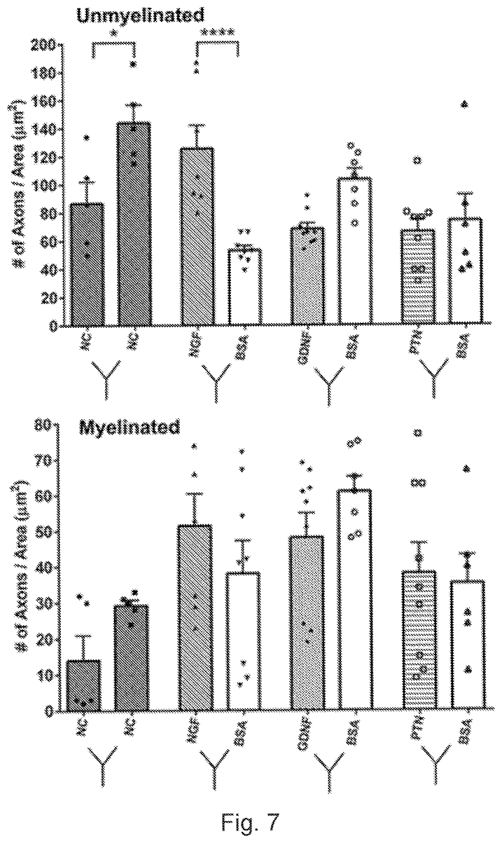

Electron microscopy evaluation of the regenerated fascicles showed normal myelinated and unmyelinated axonal composition (FIG. 6) in all treatment groups. Large myelinated axons were evident in the nerve-caps and less abundant in the NTF treatments, and the myelin thickness appears to be similar among all the groups. The number of unmyelinated axons are more evident in the NGF treatment compared to the other NTF groups. The data implies that axonal composition is differentially affected by the NTF treatments. Quantification of the fibers types revealed a significant effect by the NTF treatment in unmyelinated axon count (one-way ANOVA; P≤0.0001; F (7, 48)=7.986; R²=0.538) (FIG. 7). The NGF compartment (118.1±44.1) had significantly higher number of unmyelinated axon count compared to BSA (66.3±26.8). GDNF and PTN showed no difference in number of unmyelinated axon count when compared to their adjacent BSA compartment. The number of myelinated axons did not change significantly among the groups. Comparison of myelin thickness and fiber diameter between the experimental groups showed no difference (Supplementary FIG. 1).

3. Specific Guidance Molecules can be used to Differentially Modulate the Sensory and Motor Axons into Separate Regenerative Chambers

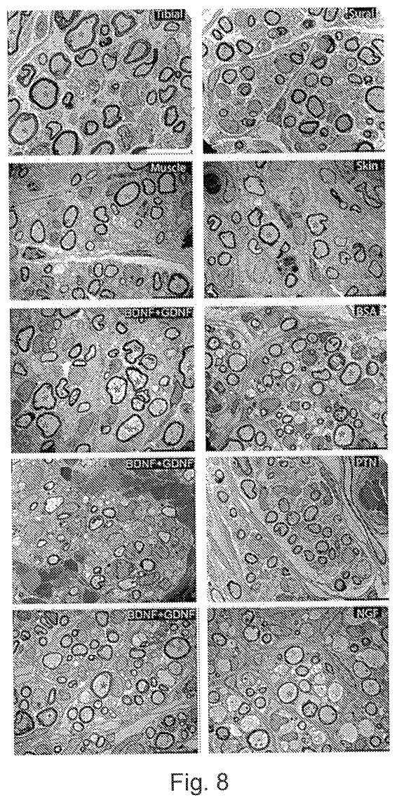

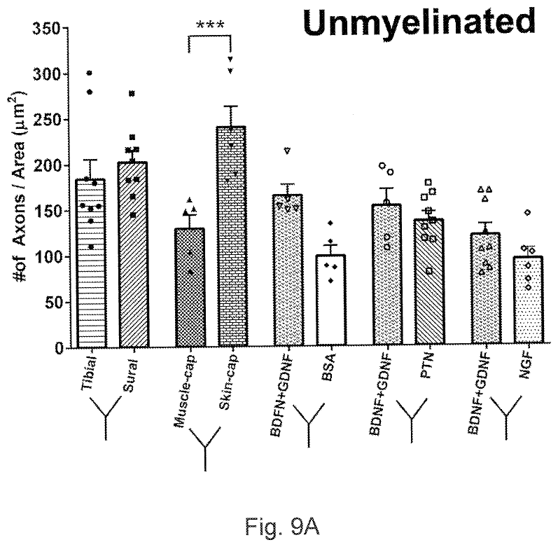

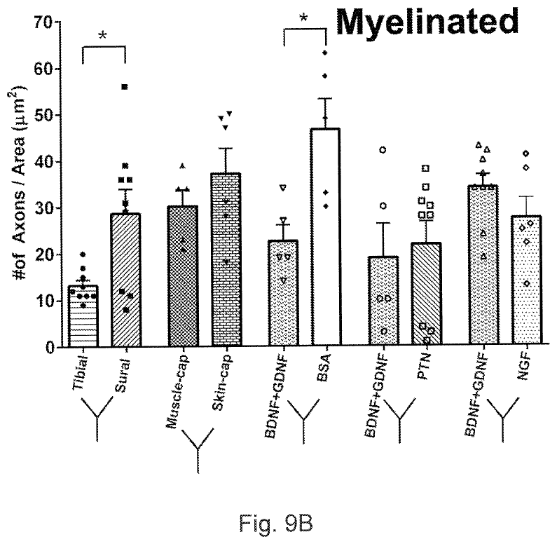

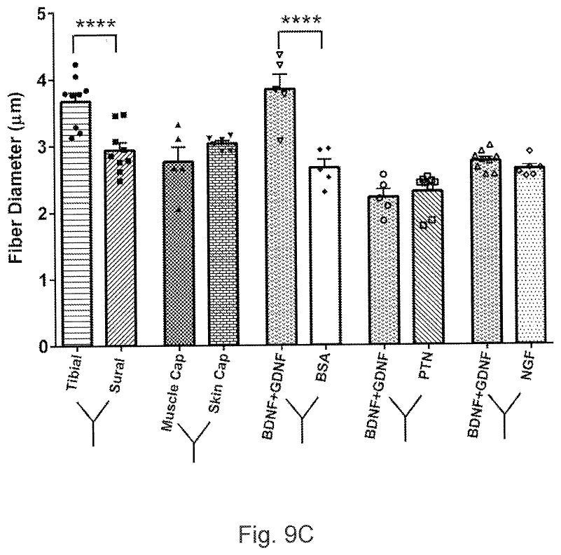

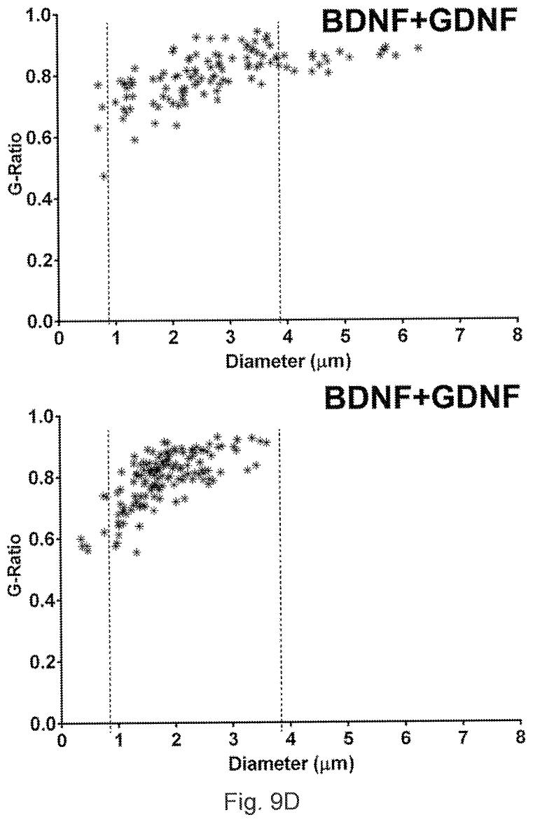

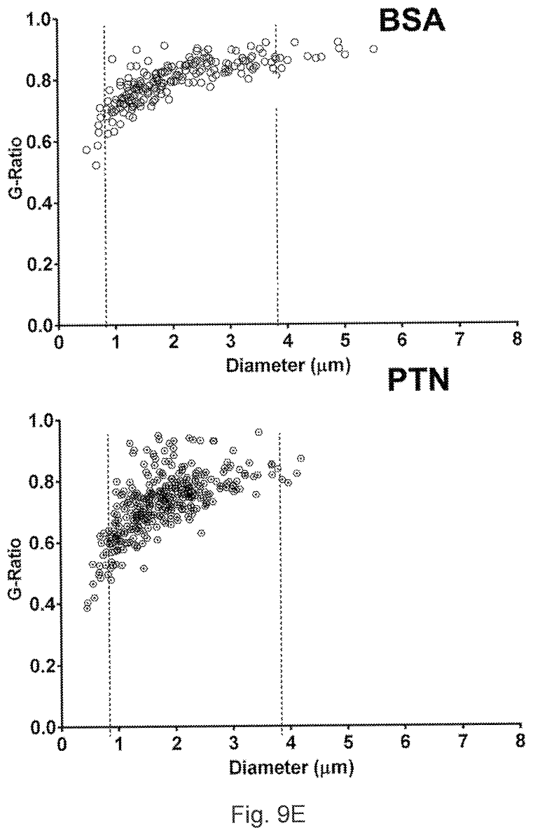

Next, the effect of presenting combination molecular guidance cues was tested in separate compartments of the Y-choice assay (FIG. 8). Large myelinated axons were evident in the tibial and muscle-cap compartments compared to sural and skin-cap targets. The BDNF+GDNF when combined in one compartment and used with BSA in the other showed a clear increase in the large myelinated axons. This effect was less evident when BDNF+GDNF was used against PTN and NGF in the other compartments. Quantification of axon morphology and composition revealed that the skin tissue had a significant effect using a one-way ANOVA (P≤0.0001; F (9, 58)=8.195; R²=0.560) (FIG. 9A). The regenerated nerve fascicle with the skin-cap showed significantly higher number of unmyelinated axon (240.0±56.3) than the muscle-cap (123.4±42.3). However, compartments with tibial-nerve or muscle-cap did not show the expected increase in the total number of myelinated axons (FIG. 9B). The number of myelinated axons in the sural-nerve (28.7±15.7) and BSA (39.0±7.6) compartment was significantly higher compared to the tibial-nerve (13.2±3.5) and BDNF+GDNF (22.6±7.9) compartment respectively (One-way ANOVA; P≤0.01; F (9, 58)=3.604; R² =0.36) (FIG. 9B). When fiber diameter was considered, there was a significant difference between the tibial- and sural-nerve group and the BDNF+GDNF and BSA compartment (One-way ANOVA; P≤0.0001; F (9, 58)=18.89; R²=0.75) (FIG. 9C). The fiber diameter of BDNF+GDNF (3.61±0.78 μm) group was significantly higher compared to BSA (2.60±0.57 μm) compartment, and comparable to the natural motor-related target of tibial-nerve (3.67±0.38). The smaller diameter axons showed higher myelination with g-ratios 0.4 to 0.7, while the larger axons were thinly myelinated with g-ratios of 0.7 to 0.9. (FIG. 9D). Comparison of g-ratio and axon diameter of the BDNF+GDNF target with BSA and PTN revealed two distinct effects. A shift to the left in the myelinated axon diameter was observed in the BDNF+GDNF-PTN group (FIG. 9) compared to the BDNF+GDNF-BSA (FIG. 9C-D).

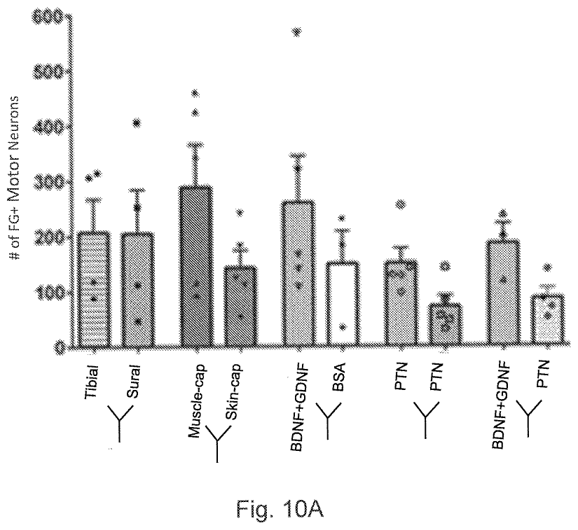

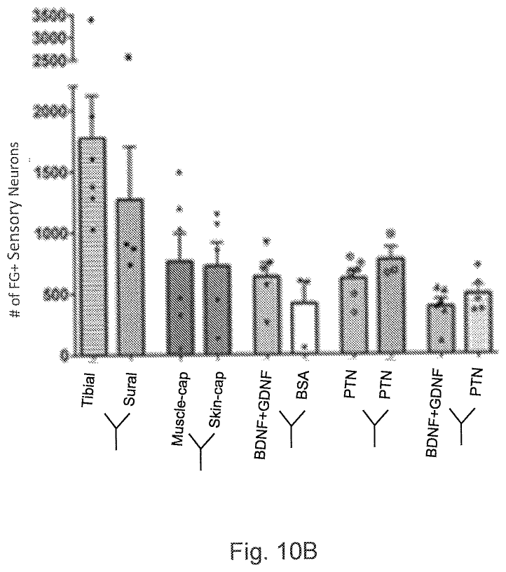

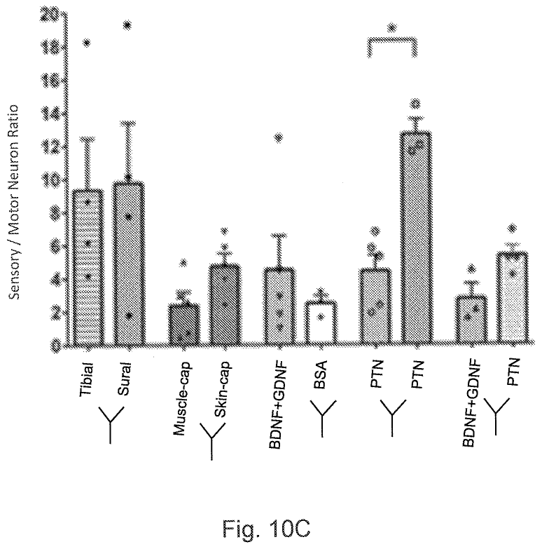

Retrograde labeling of the implants with Y-choice assay showed no specific preferential motor enrichment in the motor-mixed tibial nerve (207.0±120.8 VMN) compared to the sensory sural nerve (204.5±159.5) (FIG. 10A). In contrast, the muscle-cap attracted slightly more motor neurons (288.4±172.8) when compared to the skin-cap (142.4±71.67) without reaching statistical significance. The BDNF+GDNF compartments had increased number of motor neurons for all experimental groups versus PTN, NGF, or BSA control, however, the increase was not statistically significant by a one-ANOVA.

The total number of regenerated sensory neurons showed no significant difference among any of the treatments (FIG. 10B). A one-way ANOVA (P≤0.05; F (9, 31)=3.269; R²=0.49) revealed a significant effect in the comparison of sensory-to-motor (S/M) ratios across groups (FIG. 10C). The tibial-sural choice had comparable S/M ratios of 9.3±3.1 and 9.7±3.6 respectively. As expected the skin-cap and the NGF groups show an increased ratio compared to the muscle-cap and BDNF+GDNF counterpart, albeit not statistically significant. However, when BDNF+GDNF was presented along with PTN, the S/M ratio increased significantly in the PTN branch to 12.7±0.9, suggesting this combination would be effective in asymmetrically modulating the sensory to motor ratio.

### D. Discussion

Interfacing the damaged nerve in amputees in order to record motor intent and selectively stimulate distinct sensory-axons remains a significant challenge, despite the somatotopic organization in nerves⁵¹. Arguably, this is because most nerves are composed of a mixed number of motor, sensory, and autonomic axons. In the rat, the sciatic nerve has only 1,600 motor efferent axons from an approximated population of 27,000 axons, while 17,200 are sensory afferents and 8,200 are autonomic⁴⁷. Thus, the probability of evoking specific sensory modalities using extraneural cuff electrodes, or recording single units from motor axons using indwelling needle electrodes, is very low (P=0.059) compared to that of interfacing with mixed sensory axons (P=0.637).

Described herein is the use of nerve growth factors to selectively attract specific subsets of neurons to distinct chambers to modulate the S/M ratio. Such modulated and guided axonal regrowth could improve the possibility of motor-intent decoding and specific sensory stimulation. Herein, data demonstrates that using exogenous neurotrophic factors in vivo, the mixed rat sciatic nerve can be differentially guided into functional fascicles with normal microanatomy. When nerve-cap control segments were used distally in the arms of the Y-conduit, the number of motor neurons divided efficiently and symmetrically into two fascicles each with 50% of the motor and 29% of the sensory neurons compared to the total population in non-injured animals. The disproportional reduction in the number of DRG neurons compared to the uninjured controls is in agreement with expected cell death in this population after injury⁵⁰. The addition of GDNF into one of the compartments mediated the regeneration of motor population to levels comparable to the nerve-cap and increased the number of sensory neurons. The effect on the sensory axons can be attributed to the increased expression of GDNF receptors GFRα-1 and Ret in the large diameter neurons, and of GFRα-3 in the small diameter sensory neurons after injury⁵⁸. Compared to BSA compartments, GDNF significantly increased the total number of motor neurons 3.7 fold, confirming its ability to modulate the motor content of regenerated nerves. In contrast, neither NGF nor PTN showed a significant effect on attracting motor neurons.

With respect to sensory axons, NGF was more effective in enticing sensory neurons, as the number of unmyelinated axons in the regenerated fascicle was significantly increased compared to PTN and GDNF. This effect was specific, and is consistent with axonal sprouting rather than axonal guidance, which has been recognized in injured nerves⁵⁹. The observation that PTN, a known motor neuron growth factor^{22,39}, did not influence the number of motor neurons, but rather significantly increase the S/M ratio was unexpected. PTN is known to be up-regulated in the DRG satellite cells, Schwann cells, macrophages, and endothelial cells, which express the anaplastic lymphoma kinase (ALK) PTN receptor in the distal portion of the nerve after injury²². It is also reported that TrkA+ nociceptor neurons express the ALK receptor⁶⁰. Moreover, PTN can significantly enhance regeneration of myelinated axons across a nerve graft in adult rats, and this effect can be blocked by ALK antibody, suggesting that ALK is the receptor responsible for PTN's neurotrophic activity³⁹. The results described herein provide support for the use of GDNF in modulating the number of motor axons and of PTN in influencing sensory neurons in their path for separate compartments.

Evaluation of two different attraction Y-choice assay revealed that the tibial-sural and muscle-skin-cap showed comparable motor neuron regeneration. However, in the NTF groups, BDNF+GDNF doubled the number of motor neurons compared to those growing into PTN and NGF compartments. Consistently, the fiber diameter was found to be significantly larger in the BDNF+GDNF compared to BSA. Conversely, the S/M ratio in the PTN compartment was significantly increased. Previously, the BDNF+GDNF combination has been used in other studies to increase the number of regenerated motor neurons⁶¹. However, the present disclosure is the first to show an in vivo Y-choice assay between BDNF+GDNF and PTN to differentially modulate the axonal content in those chambers. The total number of neurons growing into the different compartments using multiple growth factors was reduced approximately 60% compared to the single growth factor treatments. This as an indication that the higher concentration of microparticles might have presented a physical barrier for nerve regeneration. Other methods to produce sustained gradients using polymeric coils in the lumen of micro channels can be used to address this issue⁴³. NTFs delivery methods can thus also be implemented with open lumen for maximal nerve growth.

While this study was successful in modulating the sensory-to-motor ratio of neurons in specific compartments, it was expected for NGF, BDNF, and NT-3 to increase the regeneration of small, medium, and larger diameter sensory neurons, respectively. However, the level of resolution on retrograde-traced sensory subtypes was not definitive. In the samples, FG masked the calcitonin gene-related peptide (CGRP) marker and thus confirmation of specific sensory subtypes was not obtained. Further, molecular guidance cues could be utilized to differentially control the number of rapid adapting (RA) and slow adapting (SA) fibers in a Y-choice nerve conduit in order to encode for specific functions such as form, texture, grip control/motion detection, tool manipulation, and hand conformation⁶². In view of the present disclosure, the goal of achieving regenerative control of mechanoceptive fibers seems feasible. The addition of selective molecular inhibitors could also be used according to the strategy of the present disclosure. Molecular repellents play a crucial role in creating patterns of somatosensory innervation by restricting growth cone extension and branching⁶³. Chemorepellents such as Semaphorins are known to restrict the path of nociceptive regenerating axons both during development and in the adult spinal cord⁶⁴⁻⁶⁶. Recently, the combination of NGF and Sema3A was used in the spinal cord, where NGF attracted TrkA+ pain fibers into the dorsal horn after rhizotomy, and simultaneous expression of Sema3A in the ventral horn restricted neurite extension into that area⁶⁷. Strategies combining attractive and repulsive cues therefore could be utilized to refine axon guidance into separate recording and stimulating compartments, as described herein. In addition to NTFs, neural cell adhesion molecules such as the polysialic acid N-CAM, L1 and N-cadherin can also be used for motor axon guidance⁶⁸.

The present disclosure demonstrates that transected mixed nerves could be regenerated and split into fascicles with differential content of motor and sensory axons. In summary, molecular guidance offers a new approach, which can be added to a comprehensive strategy to obtain directed axonal regeneration in nerve repair, or as a method to influence the axon type content in regenerative neural interfacing.

### E. References

1. Farrell, T. R. & Weir, R. F. The optimal controller delay for myoelectric prostheses. IEEE Trans. Neural Syst. Rehabil. Eng. 15, 111-8 (2007).
2. Cipriani, C., Controzzi, M. & Carrozza, M. C. The SmartHand transradial prosthesis. J. Neuroeng. Rehabil. 8, 29 (2011).
3. Tyler, D. J. Neural interfaces for somatosensory feedback: bringing life to a prosthesis. Curr. Opin. Neurol. 28, 574-581 (2015).
4. Collinger, J. L. et al. High-performance neuroprosthetic control by an individual with tetraplegia. Lancet 381, 557-64 (2013).
5. Flesher, S. N. et al. Intracortical microstimulation of human somatosensory cortex. Sci. Transl. Medicne 1-11 (2016). doi:10.1126/scitranslmed.aaf8083
6. Tabot, G. a et al. Restoring the sense of touch with a prosthetic hand through a brain interface. Proc. Natl. Acad. Sci. U. S. A. 110, 18279-84 (2013).
7. Dhillon, G. S., Lawrence, S. M., Hutchinson, D. T. & Horch, K. W. Residual function in peripheral nerve stumps of amputees: implications for neural control of artificial limbs. J. Hand Surg. Am. 29, 605-15-8 (2004).
8. Kim, Y. & Romero-Ortega, M. I. Material considerations for peripheral nerve interfacing. MRS Bull. 37, 573-580 (2012).
9. Navarro, X. et al. A critical review of interfaces with the peripheral nervous system for the control of neuroprostheses and hybrid bionic systems. J. Peripher. Nerv. Syst. 10, 229-58 (2005).
10. Tan, D. W. et al. A neural interface provides long-term stable natural touch perception. Sci. Transl. Med. 6, 257-138 (2014).
11. Graczyk, E. L. et al. The neural basis of perceived intensity in natural and artificial touch (accepted). Sci. Transl. Med. 142, 1-11 (2016).
12. Raspopovic, S. et al. Restoring natural sensory feedback in real-time bidirectional hand prostheses. Sci. Transl. Med. 6, 222ra19 (2014).
13. Oddo, C. M. et al. Intraneural stimulation elicits discrimination of textural features by artificial fingertip in intact and amputee humans. Elife 5, 1-27 (2016).
14. Davis, T. S. et al. Restoring motor control and sensory feedback in people with upper extremity amputations using arrays of 96 microelectrodes implanted in the median and ulnar nerves. J. Neural Eng. 13, 36001 (2016).
15. Grill, W. M. & Mortimer, J. T. The effect of stimulus pulse duration on selectivity of neural stimulation. IEEE Trans. Biomed. Eng. 43, 161-6 (1996).
16. Grill, W. M., Norman, S. E. & Bellamkonda, R. V. Implanted neural interfaces: biochallenges and engineered solutions. Annu. Rev. Biomed. Eng. 11, 1-24 (2009).
17. Delgado-Martinez, I., Badia, J Pascual-Font, A., Rodriguez-Baeza, A. & Navarro, X. Fascicular Topography of the Human Median Nerve for Neuroprosthetic Surgery. Front. Neurosci. 10, 286 (2016).
18. del Valle, J. & Navarro, X. Interfaces with the peripheral nerve for the control of neuroprostheses. International review of neurobiology 109, (Elsevier Inc., 2013).
19. Garde, K., Keefer, E., Botterman, B., Galvan, P. & Romero, M. I. Early interfaced neural activity from chronic amputated nerves. Front. Neuroeng. 2, 5 (2009).
20. Seifert, J. L. et al. Normal molecular repair mechanisms in regenerative peripheral nerve interfaces allow recording of early spike activity despite immature myelination. IEEE Trans. Neural Syst. Rehabil. Eng. 20, 220-227 (2012).
21. Desai, V. H. et al. Chronic Sensory-Motor Activity in Behaving Animals using Regenerative Multi-electrode Interfaces. 36th Annu. Int. Conf. IEEE Eng. Med. Biol. Soc. 1973-1976 (2014).
22. Hoke, a et al. Schwann cells express motor and sensory phenotypes that regulate axon regeneration. J. Neurosci. 26, 9646-55 (2006).
23. Brushart, T. M. et al. Schwann cell phenotype is regulated by axon modality and central-peripheral location, and persists in vitro. Exp. Neurol. 247, 272-81 (2013).
24. Hsieh, J.-H. et al. Patterns of target tissue reinnervation and trophic factor expression after nerve grafting. Plast. Reconstr. Surg. 131, 989-1000 (2013).
25. Young, H. M., Anderson, R. B. & Anderson, C. R. Guidance cues involved in the development of the peripheral autonomic nervous system. Auton. Neurosci. 112, 1-14 (2004).
26. Dudanova, I., Gatto, G. & Klein, R. GDNF acts as a chemoattractant to support ephrina-induced repulsion of limb motor axons. Curr. Biol. 20, 2150-2156 (2010).
27. Chai, G. et al. Celsr3 is required in motor neurons to steer their axons in the hindlimb. Nat. Neurosci. 17, 1171-9 (2014).
28. Wood, M. D. et al. GDNF released from microspheres enhances nerve regeneration after delayed repair. Muscle Nerve 46, 122-4 (2012).
29. Gordon, T., Sulaiman, 0. & Boyd, J. G. Experimental strategies to promote functional recovery after peripheral nerve injuries. J. Peripher. Nerv. Syst. 8, 236-50 (2003).
30. Boyd, J. G. & Gordon, T. A dose-dependent facilitation and inhibition of peripheral nerve regeneration by brain-derived neurotrophic factor. Eur. J. Neurosci. 15, 613-26 (2002).
31. Fine, E. G., Decosterd, I., Papaloïzos, M., Zurn, A. D. & Aebischer, P. GDNF and NGF released by synthetic guidance channels support sciatic nerve regeneration across a long gap. Eur. J. Neurosci. 15, 589-601 (2002).
32. Bartkowska, K., Turlej ski, K. & Djavadian, R. L. Neurotrophins and their receptors in early development of the mammalian nervous system. Acta Neurobiol. Exp. (Wars). 70, 454-467 (2010).
33. Romero, M. I., Rangappa, N., Garry, M. G. & Smith, G. M. Functional regeneration of chronically injured sensory afferents into adult spinal cord after neurotrophin gene therapy. J. Neurosci. 21, 8408-16 (2001).
34. Hu, X., Cai, J., Yang, J. & Smith, G. M. Sensory axon targeting is increased by NGF gene therapy within the lesioned adult femoral nerve. Exp. Neurol. 223, 153-65 (2010).
35. Oakley, R. a et al. Neurotrophin-3 promotes the differentiation of muscle spindle afferents in the absence of peripheral targets. J. Neurosci. 17, 4262-74 (1997).
36. Kucera, J., Fan, G., Jaenisch, R., Linnarsson, S. & Ernfors, P. Dependence of developing group Ia afferents on neurotrophin-3. J. Comp. Neurol. 363, 307-320 (1995).
37. de Nooij, J. C., Doobar, S. & Jessell, T. M. Etv1 inactivation reveals proprioceptor subclasses that reflect the level of NT3 expression in muscle targets. Neuron 77,1055-68 (2013).
38. Carroll, P., Lewin, G. R., Koltzenburg, M., Toyka, K. V & Thoenen, H. A role for BDNF in mechanosensation. Nat. Neurosci. 1, 42-46 (1998).
39. Mi, R., Chen, W. & Hoke, A. Pleiotrophin is a neurotrophic factor for spinal motor neurons. Proc. Natl. Acad. Sci. U. S. A. 104, 4664-9 (2007).
40. Lotfi, P., Garde, K., Chouhan, A. K., Bengali, E. & Romero-Ortega, M. I. Modality—specific axonal regeneration: toward selective regenerative neural interfaces. Front. Neuroeng. 4, 11 (2011).
41. Dawood, A. F. et al. VEGF Release in Multiluminal Hydrogels Directs Angiogenesis from Adult Vasculature In Vitro. Cardiovasc. Eng. Technol. 2, 173-185 (2011).
42. Punnakitikashem, P., Truong, D., Menon, J. U., Nguyen, K. T. & Hong, Y. Electrospun biodegradable elastic polyurethane scaffolds with dipyridamole release for small diameter vascular grafts. Acta Biomater. 10, 4618-28 (2014).
43. Alsmadi, N. Z. et al. Coiled polymeric growth factor gradients for multi-luminal neural chemotaxis. Brain Res. 1619, 72-83 (2015).
44. Ramanuj an, S. et al. Diffusion and convection in collagen gels: implications for transport in the tumor interstitium. Biophys. 1 83, 1650-1660 (2002).
45. Vasudevan, S., Yan, J.-G., Zhang, L.-L., Matloub, H. S. & Cheng, J. J. A rat model for long-gap peripheral nerve reconstruction. Plast. Reconstr. Surg. 132, 871-6 (2013).
46. Swett, J. E., Torigoe, Y., Elie, V. R., Bourassa, C. M. & Miller, P. G. Sensory neurons of the rat sciatic nerve. Exp. Neurol. 114, 82-103 (1991).
47. Schmalbruch, H. Fiber composition of the rat sciatic nerve. Anat. Rec. (1986).
48. Schnell, S. a., Staines, W. a. & Wessendorf, M. W. Reduction of Lipofuscin-like Autofluorescence in Fluorescently Labeled Tissue. J. Histochem. Cytochem. 47, 719-730 (1999).
49. Abercrombie, M. Estimation of nuclear population from microtome sections. Anat. Rec. (1946).
50. Schmalbruch, H. Loss of sensory neurons after sciatic nerve section in the rat. Anat. Rec. 219, 323-329 (1987).
51. Weber, D. J., Friesen, R. & Miller, L. E. Interfacing the somatosensory system to restore touch and proprioception: essential considerations. J. Mot. Behav. 44, 403-18 (2012).
52. Abernethy, D. a, Rud, a & Thomas, P. K. Neurotropic influence of the distal stump of transected peripheral nerve on axonal regeneration: absence of topographic specificity in adult nerve. J. Anat. 180 (Pt 3, 395-400 (1992).
53. Lundborg, G. Tissue Specificity in Nerve Regeneration. J. Plast. Reconstr. Surg. 20, 279-283 (1986).
54. Politis, M. J., Ederle, K. & Spencer, P. S. Tropism in nerve regeneration in vivo. Attraction of regenerating axons by diffusible factors derived from cells in distal nerve stumps of transected peripheral nerves. Brain Res. 253, 1-12 (1982).
55. Jerregård, H., Nyberg, T. & Hildebrand, C. Sorting of regenerating rat sciatic nerve fibers with target-derived molecules. Exp. Neurol. 169, 298-306 (2001).
56. Uschold, T., Robinson, G. A. & Madison, R. D. Motor neuron regeneration accuracy: Balancing trophic influences between pathways and end-organs. Exp. Neurol. 205, 250-256 (2007).
57. Romero, M. I. et al. Extensive sprouting of sensory afferents and hyperalgesia induced by conditional expression of nerve growth factor in the adult spinal cord. J. Neurosci. 20, 4435-45 (2000).
58. Allodi, I., Udina, E. & Navarro, X. Specificity of peripheral nerve regeneration: interactions at the axon level. Prog. Neurobiol. 98, 16-37 (2012).
59. Gallo, G. & Letourneau, P. C. Localized sources of neurotrophins initiate axon collateral sprouting. J. Neurosci. 18, 5403-5414 (1998).
60. Degoutin, J., Brunet-de Carvalho, N., Cifuentes-Diaz, C. & Vigny, M. ALK (Anaplastic Lymphoma Kinase) expression in DRG neurons and its involvement in neuron-Schwann cells interaction. Eur. J. Neurosci. 29, 275-86 (2009).
61. Boyd, J. G. & Gordon, T. Glial cell line-derived neurotrophic factor and brain-derived neurotrophic factor sustain the axonal regeneration of chronically axotomized motoneurons in vivo. Exp. Neurol. 183, 610-619 (2003).
62. Brushart, T. Nerve Repair. (Oxford University Press, Inc., 2011).
63. Wang, F., Julien, D. P. & Sagasti, A. Somatosensory peripheral axon guidance and morphogenesis. 7,388-394 (2013).
64. Dickson, B. J. Molecular mechanisms of axon guidance. Science 298, 1959-64 (2002).
65. Kaselis, A., Treinys, R., Vosyliute, R. & Satkauskas, S. DRG axon elongation and growth cone collapse rate induced by Sema3A are differently dependent on NGF concentration. Cell. Mol. Neurobiol. 34, 289-296 (2014).
66. Tessier-Lavigne, M. & Goodman, C. S. The Molecular Biology of Axon Guidance. Science (80-.). 274, 1123-1133 (1996).
67. Tang, X.-Q., Tanelian, D. L. & Smith, G. M. Semaphorin3A inhibits nerve growth factor-induced sprouting of nociceptive afferents in adult rat spinal cord. J. Neurosci. 24, 819-27 (2004).
68. Franz, C. K. Polysialylated Neural Cell Adhesion Molecule Is Necessary for Selective Targeting of Regenerating Motor Neurons. J. Neurosci. 25, 2081-2091 (2005).

### EXAMPLE 2

### Asymmetric Nerve Growth

### A. Introduction

The human hand is populated by an estimated 17,000 touch sensing receptors in the skin that provide information about small slips, skin deformation and limb position [24], [157]. These individual nerve fibers are classified based on conduction velocity and axon diameter which include: Type I included muscle spindles (Ia) and tendon organs (Ib;12-20 μm AD and 100 m/s CV), Type II that are mehanoceptors (Aβ; 4-12 μm AD, 60 m/s CV), Type III are delta nociceptors (Aδ; 1-11 μm AD, 60 m/s CV), and Type IV C-pain fibers (0.5-1 μm AD, 1-2 m/s CV) [23]. Moreover, there is overlap in axon diameters among the different modality types resulting in no distinction between proprioceptive and large mechanoceptive afferents [25], [158], [159]. Also, most somatic nerves contain two types of motor axons (α and γ) and five different types of sensory afferents including Aβ proprioceptive axons, high-threshold mechanoceptors (HTMRs), low-threshold mechanoceptors (LTMRs), slow-conducting C-nociceptors, and myelinated pain Aδ fibers, all of which are mixed at various proportions and quantities in different nerves [23], [24], [51]. Clearly, interfacing and stimulating a specific sensory modality from mixed sensori-motor somatic nerves presents a great challenge. This is even further complicated by the fact that large myelinated axons (i.e., proprioceptive and motor) are depolarized with smaller electrical currents, while smaller diameter neurons (i.e., pain fibers) require larger depolarizing stimuli. Thus, when stimulating the small caliber fibers, one can expect to non-specifically recruit large-size axons as well [26], [27], [160], [161].

In addition to attractants, repellents are also key mediators for axon development and guidance. The Semaphorins family of guidance cue are a large class of protein that are most widely studied. During development semaphorins are present as either soluble or membrane bound and emit a long or short range repulsive action. They are large proteins consisting of approximately 500 amino acids and divided into eight subclasses and with their activity being mediated by two receptors, plexin and neuropilin [168]. Most classes of semaphorins bind to plexin directly, while class 3 (Sema3A) binds to neuropilin first with its complex activating plexin and leading to growth cone collapse [40], [169]. In the adult PNS, class 3 semaphorins are upregulated following peripheral nerve injury [170], and interestingly, act as both attractive and repulsive, hence their role has yet to be fully elucidated. However, the role of Sema3A, a member of the semaphorin family, has been shown to induce turning and growth cone collapse of sensory DRG neurons in vitro [29], [171]-[173]. Furthermore, in vivo application of NGF attractant in the dorsal column of the spinal cord and exogenous presence Sema3A on the ventral side induced sprouting of NGF responsive axons to the dorsal side while inhibiting its extension towards the ventral side of the spinal cord [30], [101].

Described herein by the present inventors is the incorporation of both attractive and repulsive cues as a means to refine axon guidance and regeneration. The present Example describes a study to determine whether the application of both attractive and repulsive cues can further modulate the sensory to motor ratio (S/M) and improve the enrichment of axon subtype given two different modality attractants. Herein, it is demonstrated that Semaphorin 3A inhibits small diameter axons in a choice assay with BDNF and NGF molecular attractants.

### B. Materials and Methods

1. PLGA encapsulation of Sema3A

Recombinant human Semaphorin-3A (Sema3A, 87.3 kD) (Novoprotein, Summit, N.J.) protein was encapsulated in biodegradable poly(DL-lactic-co-glycolic acid (PLGA) microparticles using a double emulsion method. Briefly, PLGA 50:50 (Lakeshore Biomaterial, St. Louis, Mo.) was dissolved in dichloromethane (DCM) 200 mg/ml (Sigma-Aldrich, St. Louis, Mo.), and mixed with aqueous solutions of 20 ug/ml of recombinant sema3A protein. This solution was then added to polyvinyl alcohol (20 mg/ml) and emulsified. The MP solution was stirred for 1-2 hours to remove excess DCM, centrifuged at 4000 rpm for 15 minutes to pellet the particles and separated from the supernatant. The resulting MPs were transferred to −20° C. for 2, to −80° C. overnight, freeze dried for 48 hours and stored at −20° C. until used. Loading efficacy was calculated at 67±5%.

### 2. In Vitro Y-Template Fabrication

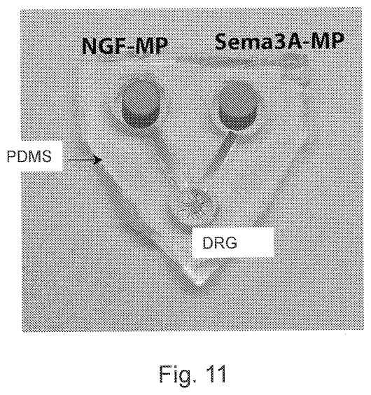

A polydimethysiloxane (PDMS) template in the shape of a “Y” was fabricated to test the bioactivity of the Sema3A-MP. Briefly, the elastomer and the curing agent was mixed in a 10 to 1 ratio, mixed, and cured in the oven at 60° C. for 2 hours. Using a 6.0 mm biopsy punch, two holes were made approximately 1.5cm apart. Another hole, 1.5cm below was also made using a 5.0 mm biopsy punch creating a 60° angle. Finally, ˜2 mm canals were cut joining each of the 6.0 mm holes connecting to the 5.0 mm hole (FIG. 11). The PDMS Y-template was then attached to a glass cover slip following plasmapheresis treatment and sterilized using ethanol and UV radiation.

### 3. Sema3A-MP Bioactivity Assay

NTF-MP containing NGF and Sema3A were mixed in 30 μl of atelomeric chicken collagen (85% type I, 15% type II; Millipore; Temecula, Calif.) and added to the 6.0 mm compartments. The Y-templates were incubated at 37° C. with 5% CO₂ for 15 minutes to allow gelation. Embryonic (E15-E18) mice pups were dissected and whole dorsal root ganglia (DRG) were collected in in L-15 Medium (Leibovitz). The DRGs were cleaned of connective tissue and placed in poly-D-lysine (PDL) coated Y-template cell chamber suspended in 10 μl of atelomeric chicken collagen (FIG. 11). The explants were incubated at 37° C. with 5% CO₂ for 15 minutes to allow gelation before adding 200 μl of Neurobasal A media (Sigma Aldrich) supplemented with 0.5% penicillin/streptomycin.

### 4. DRG Choice Assay Quantification

Seven days following DRG regeneration in the Y-template, the neurite outgrowth site was imaged using the bright field on a Nikon A1R confocal microscope system (Nikon, Inc.). The images were acquired at 20× magnification and the individual images were stitched together within the Nikon's ND Elements software (Nikon, Inc.). Individual axons were traced from the boundary of the DRG to the axon terminal using ImageJ analysis software. Axonal length and degree of turn from each compartment was quantified from 3 DRG treatments.

5. Y-tube implantation and Analysis

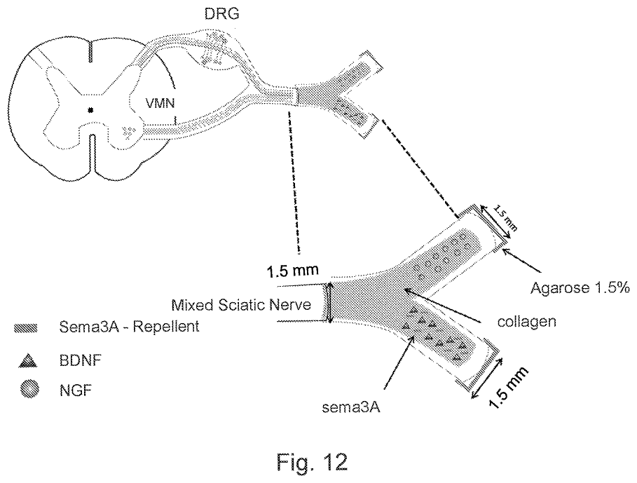

Twenty four adult female Lewis rats were included in the study. The control group received BDNF-MP in one arm and the other NGF-MP (n=12; Retrograde label 6 animals per NTF arm). The experimental group received a combination of BDNF-MP+Sema3A-MP in one arm and NGF-MP in the other (n=12; Retrograde label 6 per NTF arm) (FIG. 12). The surgical implantation, retrograde labeling, CNAP analysis, and quantification were performed similarly as described previously. All animal procedures were performed in accordance with the guidelines of the Institutional Animal Care and Use Committees of the University of Texas at Dallas.

### 6. Statistical Analysis

Changes in axonal length and turning in vitro were analyzed using student t-test. In vivo comparison between experimental groups were compared using one-way ANOVA and Bonferroni's ad-hoc multiple comparison test using Prism 6 software (GraphPad Software Inc.). A p≤0.05 was considered statistical significant. The data is presented as the mean±standard error of mean.

### C. Results

### 1. Axonal Turning in the Presence of Sema3A In Vitro

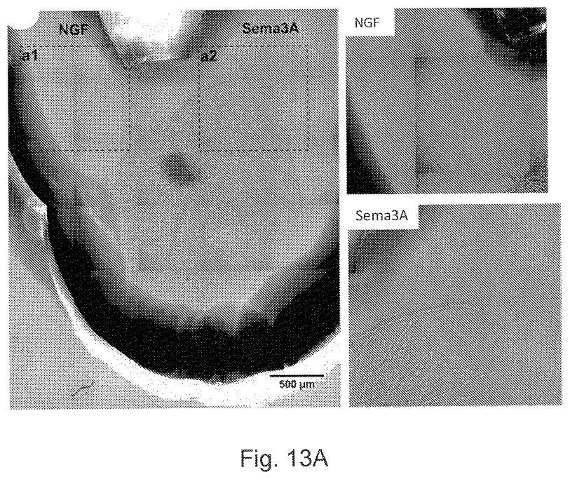

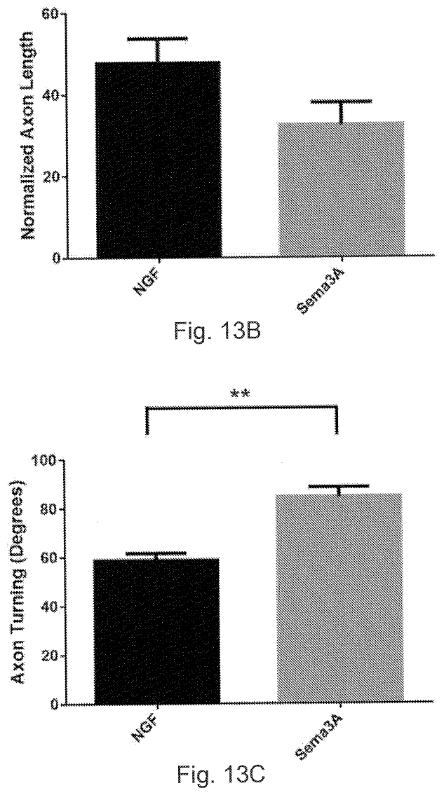

The bioactivity of the Sema3A-MP was tested on DRG explants in a choice assay. The inhibitory effect of the Sema3a in deterring axonal extension towards the chamber was observed (FIG. 13A (a1 & a2). A student t-test analysis showed a statistical significant effect in the axonal turning for NGF versus Sema3A compartment (P≤0.01, Student t-test). The mean axonal length towards the NGF (43.9±21.9%) compartment was higher compared to the Sema3A side (29.9±19.7%) without reaching significance. Similarly, axonal turning was significantly greater in the Sema3A compartment (83.8±32.5 degrees) while the NGF compartment was limited (60.2±23.0 degrees) (FIGS. 13B & 13C).

### 2. Sema3A Retains Functionality of the Regenerated Nerves

CNAP analysis from the regenerated Y-nerves in the presence of a repellent retained electrical functionality. Multiple peaks were observed following the activation of all fiber types and the latency was measured from the start of the stimulus. The conduction velocity (CV) was calculated following spike triggered averaging, and the values were categorized into slow (≤5 m/s), medium (5<x≤30 m/s), and fast (>30 m/s). One hundred percent of the regenerated nerves fascicles show electrical competency with the number of peaks ranging from 1-3. No significant difference in the number of the peaks were observed between the groups.

TABLE 3
CNAP peaks incidences observed within the velocity ranges.

| | Fast | Medium | Slow |
|---|---|---|---|
| | (>30 ms⁻¹) | (5 < x < 30) | (≤5 ms⁻¹) |
| BDNF (6) | ++++ | ++++++ | +++++ |
| NGF (6) | ++++ | ++++++ | ++++ |
| BDNF/Sema3a | ++ | +++++++ | +++++ |
| (6) | | | |
| NGF (6) | + | ++++++ | ++++++ |

‘+’ indicates one peaks observed within the range.
(#) indicates number of animals used to obtain CNAP response.

3. Sema3A does not Modulate the S/M Neuron Ratio

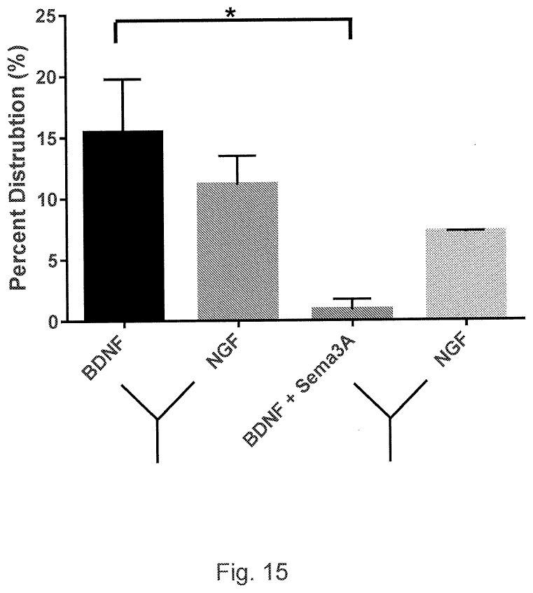

FG+ motor neurons and DRG sensory neurons were quantified from the ipsilateral spinal cord and the L4 and L5 DRGs. The number of regenerated motor neurons ranged from 130-170 using BDNF and NGF attractants, and the number of VMN in the regenerated nerve fascicles were similar in the BDNF vs. NGF and BDNF+Sema3A vs. NGF. The number of regenerated sensory neurons in the BDNF vs. NGF and BDFN/Sema3A vs. NGF groups were quantified and showed similar distribution in both experimental groups (FIG. 15). Additionally, DRG perikaryal size was also quantified and categorized into small (<300 μm²), medium (300 μm²<x<700 μm²), and large (700 μm²<). The small DRG cell size showed all groups with greater than 20% distribution without reaching significance. The medium and large size DRG cell body size ranged from 40-55% and 17-32% respectively. Both groups showed no statistical difference among the arms and across the group.

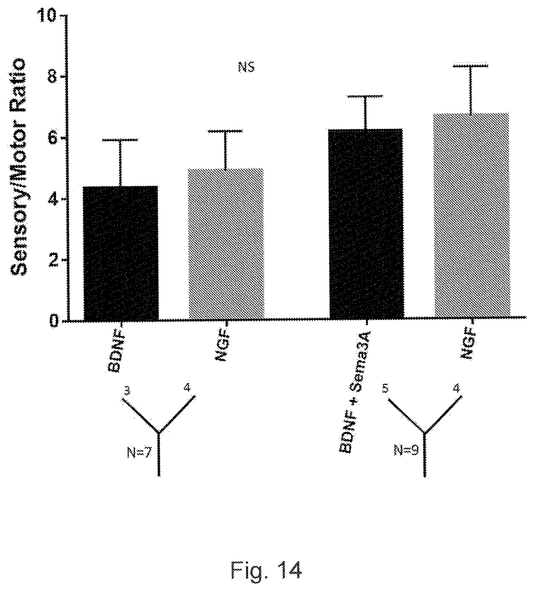

The sensory/motor neuron ratio was determined from the positively labeled VMN and DRG sensory neuron in each compartment (FIG. 14). The mean S/M ratio between the experimental groups ranged from 4.3-6.6. Comparison of the individual fascicles in each group showed no significant effect. Additionally, the ratio of BDNF arm (4.3±2.7) compared to the BDNF+Sema3A (6.1±2.6) also showed no difference in the presence of a molecular repellent.

### 4. Axon Morphology Analysis in the Presence of Sema3A

A subset (n =3 Y-nerves per compartment per group) of the individual arms of the regenerated Y-nerves were analyzed for axon type composition and myelination. Evaluation of each Y-nerve showed the presence of normal myelinated and unmyelinated axons with intact perineurium and epineurium. The number of unmyelinated and myelinated axon count showed no statistical significance within the arms of the group and across experimental groups. Similarly, myelinated axon count showed no difference between the arms and across experimental groups.

5. Sema3A Shows Inhibitor Effect of Myelinated Axons less than 1 μm

G-ratio as a function of axon diameter was plotted for each regenerated NTF-MP arm, and the relation was best fit using linear regression. The BDNF vs. NGF group was showed a steeper slope for NGF with higher myelination for small diameter axons and less myelination for higher axon diameter compared to the BDNF arm. The BDNF+Sema3A vs. NGF group had a similar fit; however, a limited number of small caliber axons was observed in both the arms of the Sema3A group (<1 μm diameter). Percent distribution of axons less than 1 μm showed a significant effect (˜95% decrease) when compared to the BDNF arm without Sema3A using a one-way ANOVA (P≤0.05; F=4.35; R²=0.65). The NGF arm in the Sema3A group had a 41.7% decrease compared to the NGF arm without the Sema3A without reaching statistical significance. Percent distribution of myelinated axon diameter within the medium (1>x≥4 μm) and large diameter (>4 μm) showed no difference.

### D. Discussion

In this Example a combination of chemoattractant and chemorepellent was tested to improve the efficacy of axon subtype enrichment. This was tested using semaphorin3A, a repulsive cue for NGF+ axons that express the receptor neuropilin [169], [172]. Sema3A was introduced into the adjacent compartment of the NGF to direct small diameter axons away from the BDNF+Sema3A chamber. The exogenous application of the Sema3A−MP resulted in a significant reduction in the number of small axons less than 1 μm in diameter. This was verified using axon morphological measurements. However, the effect of Sema3A was also observed in the adjacent arm of the Y-conduit, resulting in a similar response but to a lesser effect in the NGF compartment. Additionally, the presence of Sema3A showed no effect on other axon subtype regeneration or functionality as both experimental groups had comparable values. These findings demonstrate that mature sensory afferents in the peripheral nerve retain their responsiveness to the Sema3A and can have a trophic effect in a choice assay without distal targets.

### 1. Reduced Sprouting in the Presence of Sema3A

During development growth promoting molecules enable axonal elongation to their appropriate target. The selective expression of repellent cues further mediates that accuracy in path finding for the developing axons [29]. In the developing spinal cord, NGF positive C-fiber (nociceptors) respond to Sema3A, and prevent improper targeting in the dorsal laminas by restricting growth of C-fibers past the designated location. The presence of Sema3A in the adult similarly responds to mature NGF+ sensory afferent and has been shown to impede axonal sprouting in the spinal cord [30]. In this Example, a reduced number of small diameter axons following Sema3A expression was observed in the Y-conduits in the peripheral nerve. Scatter plots of g-ratio as a function of axon diameter revealed limited number of axons less than 1 μm in diameter in both the NTF compartments. Conversely, the non-Sema3A group showed no inhibition of small diameter axons in either of the NTF compartments. The reduction in the small diameter myelinated axons suggest that they are Aδ fibers. In previous studies, the expression of Sema3A induced a repulsive effect on small Aδ fibers in the adult cornea [174]. However, this difference in the number of small fibers in the axon morphometric analysis did not show a similar effect in the distribution of DRG neurons. A plausible explanation in the comparable size distribution yet limited small diameter axons could be the inhibition of collateral sprouting from the NGF+ axons. Interestingly, the lack of difference observed in the DRG size distribution suggests that the NTF exerted a strong enough effect to induce axonal elongation, but Sema3A was present to minimize axonal sprouting. A delicate balance between the concentration of both the attractant and the repellent can be important, as demonstrated when higher NGF expression can overcome the inhibitory effect of Sema3A [30]. Additionally, the small diameter axons observed in the NGF compartment are also indicative of the cross-diffusion by Sema3A to the adjacent compartment.

Axonal sprouting in an amputee model has been previously reported by calcitonin gene-related peptide (CGRP) positive cells (marker for small caliber peptidergic nociceptors) and isolectin-B4 positive cells (marker for small caliber non-peptidergic nociceptors) [126], [175]. In the current Example, FG+ cells in the DRG were stained using CGRP marker to find a correlation. However, the CGRP antibodies failed to stain FG+cells in all experimental groups, but axonal staining within the DRG was observed (data not shown). A possible explanation is the presence of FG in the cytoplasm or on the cell surface is limiting the binding of the antibody. Additionally, the number of unmyelinated axon count in both the BDNF and BDNF+Sema3A compartment was comparable. This could be the presence of non-peptidergic IB4 positive DRG neurons, which are unaffected by Sema3A. These particular nociceptors change from NGF+to IB4 expressing during developmental stages. The number of unmyelinated axon count in the NGF arm of the non-Sema3A group was higher compared to the NGF arm of the Sema3A group which could suggest the effect of Sema3A on the peptidergic axon leading to a lower unmyelinated axon count.

2. Limited Specificity with Two Attractants

This Example aimed to increase sensory axon subtype specificity using two specific NTF: BDNF to attract mechanoreceptors and NGF to entice the regeneration of thermoceptive/nociceptive neurons. However, with single NTFs in each compartment, the specificity was still not attained. The motor neuron enrichment from the BDNF compartments saw ˜55% reduction compared to BDNF vs. BSA group. Small caliber DRG sensory neuron enticement with NGF was also reduced by ˜50% compared to the single NTF group from Example 1. These observations suggest the presence of NTFs in both compartment of the Y-conduits, in some cases, results in limited specificity. 3. Conclusion

This Example demonstrates that exogenous chemorepellents present in the developmental stages can be implement following adult peripheral nerve injury to induce an inhibitory effect. Conversely, this inhibitory effect was not observed in motor neurons as they have been shown to moderately upregulate neuropilin and Plex-A1 mRNA level [176]-[178]. The inhibitory effect of Sema3A on a small axon subtype validate its use to further improve upon the sensory/motor ratio for developing better neural interfaces.

### EXAMPLE 3

### Combination of Pleiotrophic and Neurotrophic Factors

In the present non-limiting Example it is demonstrated that multi-luminal conduits with pleiotrophic support enable regeneration across critical nerve gaps.

### A. Summary

Nerve autografts remain the preferred surgical method to bridge peripheral nerve gap injuries longer than 3 cm, though it requires sacrifice of donor nerves and risks deleterious morbidity. Alternatively, hollow conduits, allografts and multi-luminal scaffolds are used for short gap repair, and all have shown increased effectiveness when supplemented with neuron growth factors. However, neuronal support is insufficient to match the recovery observed with autologous nerve grafts. This Example investigated the synergistic effect of trophic supplement, designed to target several cell types at the injury site, in improving nerve regeneration across critical nerve gaps. This example demosntrates that nerve regeneration could be enticed across a 3 cm long gap by sustained release of vascular endothelial growth factor (VEGF) or pleiotrophin (PTenn.) in the lumen of collagen-filled hydrogel microchannels. In vitro evaluation of neurotrophin and pleiotrophic support in spinal cord and dorsal root ganglia revealed the increased potency of combined PTN and glial-derived neurotrophic factor (GDNF) in promoting sensory and motor axonal growth. The PTN/GDNF combination successfully mediated nerve regeneration across a 4 cm gap, axon maturation distal to the repair site, and recovery of toe spread function, albeit with delayed re-myelination. These results reveal the benefit of pleiotrophic support in long gap injury repair and underscore the need for providing re-myelination support distal to the injury site.

### B. Introduction

Despite the regenerative capacity of the adult peripheral nervous system and the routine repair of small nerve defects (<3 cm) using decellularized nerve grafts or nerve conduits, the surgical repair of critical lesions larger than 4 cm remains a significant challenge, with limited expectations for recovery of function even with the use of autologous grafts^{1,2}. The poor success with long nerve gaps can be attributed to a number of factors: gap distance, neuronal intrinsic regenerative capacity, growth substrate, trophic support, axon guidance errors, and re-innervation of dysfunctional targets^{3,4}. Despite the complex nature of this problem most studies propose the use of hollow nerve conduits, processed allografts, and/or single growth factors as repair strategies, with minimal success⁵. Modification of the luminal architecture to increase the growth surface area, addition of extracellular matrix molecules (ECM) such as collagen or fibronectin, and cellular and growth factor support, are reportedly more efficient compared to hollow nerve guides, but fail to match the regenerative capacity offered by autologous grafts⁶.

The advantage of isogenic nerve implants seems to be the release of neurotrophic factors by the denervated Schwann cells⁷, which, in sensory nerves, express insulin growth factor-1 and vascular endothelial growth factor (VEGF), hepatocyte-derived growth factor (HGF), nerve growth factor (NGF), and brain-derived neurotrophic factor (BDNF), whereas the heparin-binding protein pleiotrophin (PTenn.) is expressed primarily in motor fibers. Others neurotrophic factors, such as glial-derived neurotrophic factor (GDNF), insulin growth factor-2 (IGF-2), ciliary neurotrophic factor (CNTF), fibroblast growth factor-2 (FGF-2) and neurotrophin 3 (NT-3) increase after injury in all nerve types^{8,9}. NGF, BDNF and NT-3 are able to increase axonal regeneration both in vitro and in vivo^{10,11}. The stimulatory effect of neurotrophins is mediated through specific tyrosine receptor kinases (trk) and p75 receptors, and activation of the extracellularly regulated kinase (Erk1/2) by the trk receptors endows peripheral neurons with enhanced regenerative capacity¹². However, when applied to the transected peripheral nerve, the beneficial effect of these growth factors is less clear. Previous regenerative strategies are neurocentric and focus primarily on stimulating axonal growth.

This Example illustrates that a growth factor milieu that stimulates regeneration of both non-neuronal and neuronal populations in the peripheral nerve constitutes a more robust and efficient method for repairing critical long gap nerve injuries. This Example describes the effect of multi-luminal delivery of single growth factor (VEGF or PTenn.) for nerve injury at the upper gap limit of simple conduit repair (i.e., 3 cm). This Example further describse the synergistic effects of neurotrophins, glial-derived growth factors and pleiotrophins on motor and sensory axonal growth. This Example illustrates a synergistic effect of GDNF-PTN in vitro, and confirms the beneficial effect of this combination in mediating nerve regeneration across a 4 cm critical nerve gap.

### C. Results

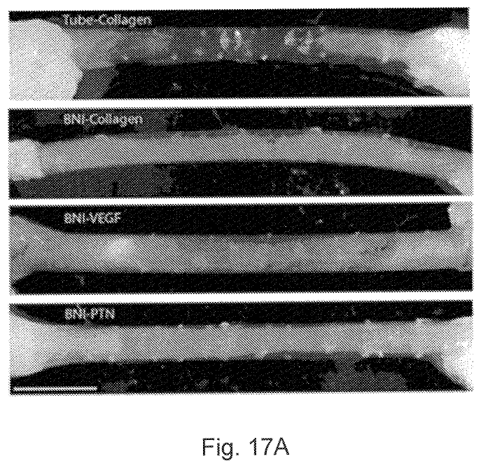

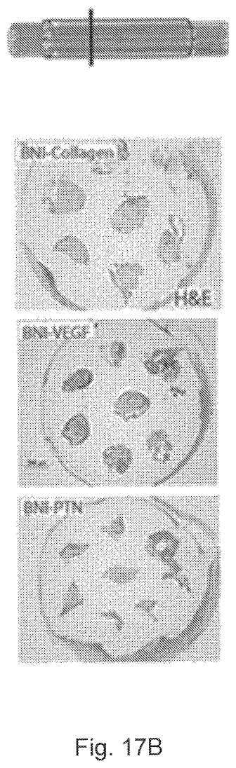

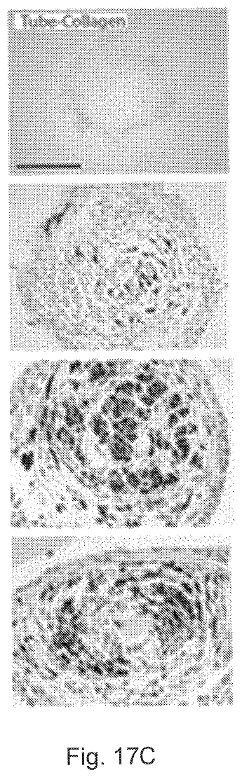

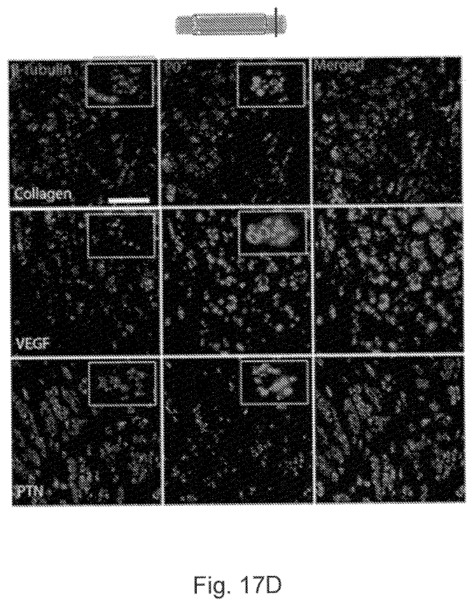

The effect of PTN or VEGF in mediating nerve regeneration across a 3 cm-long nerve gap was evaluated. Gross evaluation of the regenerated nerves 12 weeks after implantation demonstrate successful growth across the implant in animals with either a collagen-filled BNI, or in BNIs with VEGF or PTN sustained release. None of the animals implanted with nerve conduits filled with collagen only (empty tube) showed successful reconnection to the distal end (FIG. 17A). Histological evaluation of the regenerated tissue 1 cm from the proximal end, showed robust axonal regeneration that filled the 7 hydrogel microchannels in the lumen on the BNIs (FIG. 17B). Evaluation of neurofilament-positive fibers in the microchannels increased axonal density in BNIs with growth factor release confirmed H & E staining (FIG. 17C), and quantification of the number of axons per channel confirmed a significant three-fold increase in the number of axons induced by both PTN and VEGF (FIG. 17E). Immunofluorescence visualization of β III tubulin and P0 in the nerve segment distal to the implant confirmed that a significant number of both myelinated and unmyelinated axons were able to grow past the BNIs, compared to collagen-filled nerve conduits (FIG. 17D). Within those repaired with BNIs a trend was observed suggesting that PTN mediated a larger regenerative effect compared to VEGF and collagen filled BNIs, however this change did not reach statistical significance (FIG. 17E).

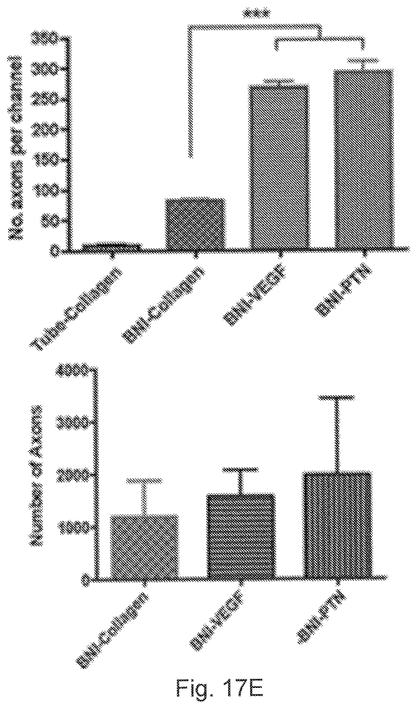

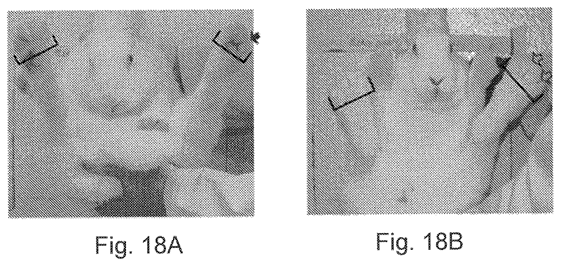

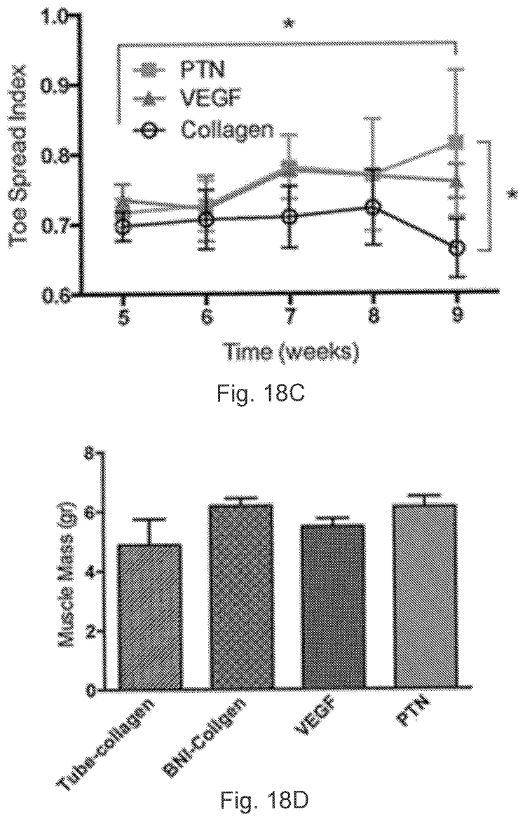

The significance of the observed regeneration was indicated by the recovery of toe-spread function in the BNI implanted animals. All animals were unable to show digit abduction up to 6 weeks after injury repair (FIG. 18A). By week 7, those implanted with VEGF or PTN eluting microparticles showed improvements in the ability for toe spreading, which increased significantly in those implanted with BNI-PTN by week 9 (FIGS. 18B, 18C). However, none of the groups showed a significant recovery in the total muscle mass at this time (FIG. 18D). The result indicated that PTN has nerve-promoting activity on non-critical gap injuries without inducing abnormal proliferation of cells at the injury site.

### 1. PTN-Meurotrophin Synergistic Effect in Neuron Regeneration and Axon Length In Vitro

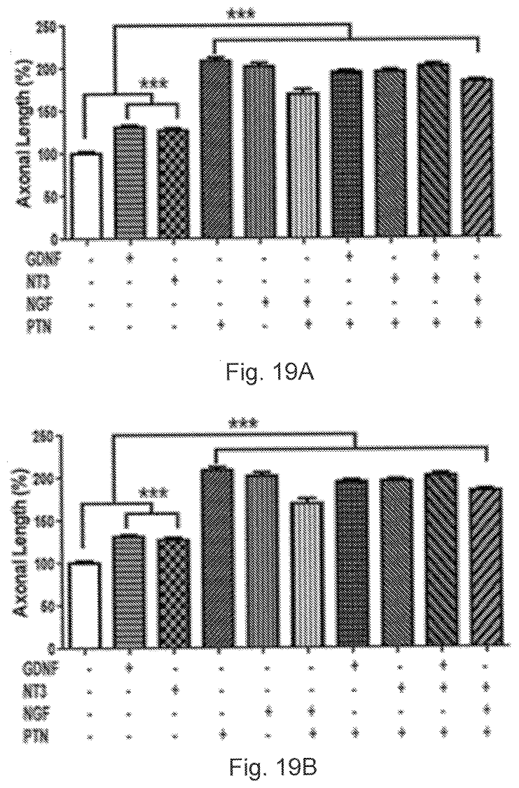

The relative potency of PTN compared to neurotrophins is unknown. This Example used neonatal DRG and spinal cord (SC) explants supplemented with growth factors to determine the regenerative capacity of single growth factors and whether synergistic effects could be elicited by a combinatorial treatment of molecules with neurotrophic and pleiotrophic actions. In the DRG cultures, both the number of axons elongating from the sensory ganglia and the maximum axonal length were influenced by the treatments. Qualitatively, DRGs exposed to single growth factors showed longer axons (GDNF<NT-3<NGF<PTenn.) compared to controls lacking growth promoting supplements. Both the axonal length and density was clearly potentiated when PTN was combined with neurotrophins (NT-3<GDNF<NGF) compared to that of each growth factor alone (FIG. 19A). The number of DRG elongating axons stimulated by GDNF (130.5±2.5 μm) or NT3 (126.7±4.1 μm) was not significantly different compared to DRG explants that received no growth factor support (100±2.3 μm). In contrast, those supplied with NGF (201.8±4.1 μm), PTN (208.4±3.89 μm) or PTN-GDNF (194.5±2.47 μm) significantly doubled their axon length (p<0.001; FIG. 19B). Evaluation of axonal density revealed a mild effect of single growth factors, whereas the combination of PTN either with GDNF, NT-3 or NGF showed a 2-fold increase compared to PTN alone. The addition of three factors in combination yielded no further significant increase in axonal growth or axonal density in DRG explants. To determine the effect of growth factors on axonal growth from motor neurons recombinant BDNF, GDNF, PTN and their combinations were added onto neonatal SC explant cultures (Sup.1A). Compared to controls with no growth factors, those with BDNF and PTN, but not GDNF, showed a trend for increased axon numbers. Interestingly, less growth was observed when BDNF and GDNF were combined, compared to the individual growth factors, whereas the combination of PTN either with BDNF, GDNF or PTN-BDNF-GDNF showed a significant 3-fold increase in axon numbers (Sup.1 C). Evaluation of axon growth in the GDNF-PTN group showed an increase by 50% (184.8±11.3) compared to the negative control (100.2±6.9; p<0.05), or individual PTN, GDNF and BDNF (136±6.1, 113.4±9.4, and 120±4.1 respectively). A significant effect was observed in axonal length when BDNF, GDNF and PTN were combined (Sup.1). However, considering both motor and sensory the data indicated a synergistic effect of PTN with GDNF on sensory neurons for maximal axonal length and density, and in the number of motor axons from the SC, and suggested the use of this dual growth factor treatment, as a promising strategy for the repair of a 4 cm critical nerve gap.

2. A BNI with Multi-Luminal PTN-GDNF Mediates Nerve Regeneration Across a 4 cm Gap.

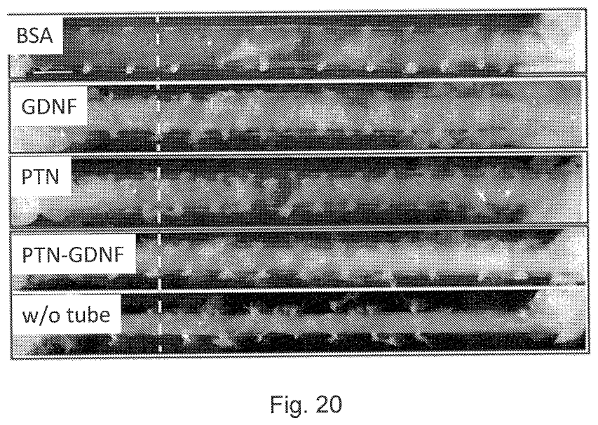

When nerve regeneration was evaluated in animals with a 4 cm nerve injury gap repaired with BNIs filled with either with GDNF, PTN or a PTN-GDNF MPs 5 months after implantation, they showed tissue growth along the BNIs connecting the proximal and distal nerve stump. Qualitative evaluation of the regenerated nerves was based on the apparent tissue density and indicated that the amount of nerve growth was better in GDNF<PTN<PTN-GDNF compared to the BSA controls (FIG. 20). Double immunofluorescence of tissue sections taken 1 cm from the proximal end, showed larger myelinated axons in the cut-resuture control compared to those with BSA, where most of the β-tubulin positive axons were not labeled with the P0 myelin marker. Those with sustained release of GDNF showed larger axons but with limited myelination. Conversely, those with PTN and with PTN-GDNF showed large myelinated axons and clusters of unmyelinated axons similar to those observed in the cut-resuture controls.

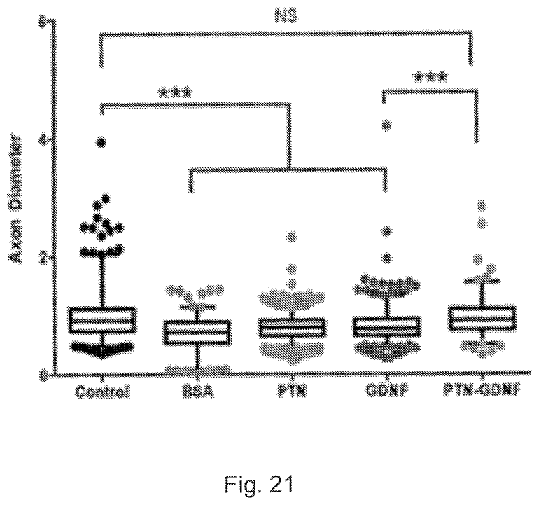

Electron microscopy was used to evaluate the number of axons that regenerated 2 mm distal to the implant. In the cut-resuture control large, medium and small myelinated axons were observed, and some unmyelinated axons in the process of being segregated from the Remak bundles (FIG. 21). In sharp contrast, those repaired with BNIs showed mostly unmyelinated axons of small diameter all within Schwann cell Remak bundles. Compared to the BSA group, those with GDNF and PTN showed an apparent increase in the number of axons and in axon diameter (FIG. 21). Large diameter axons were also observed in the PTN-GDNF group within Remak ensheathing; however, some re-myelination of large axons was also observed (FIG. 21). Evaluation of the axon diameter distribution in these samples revealed that the mean fiber size in the cut-resuture control was 1.01±0.47 μm, with large axons up to 7 μm in diameter. Those with BSA-filled BNIs averaged axon diameter of 0.70±0.30 μm and no axons larger than 2 μm were observed. The addition of MPs releasing GDNF or PTN increased the mean axon diameter to 0.84±0.48 μm, and 0.80±0.21 μm, respectively. The larger axons in these two groups measured 7 μm and 2.3 μm, respectively. In contrast, animals implanted with BNIs releasing a PTN-GDNF combination showed an average axon diameter (0.96±0.35) μm similar to the cut-resture control and statistically larger compared to the BSA and individual growth factor treatments (FIG. 21). This result indicated that while axons did regenerate across the BNI the resident Schwann cells failed to support the maturation and re-myelination of those with luminal BSA, and that while those with either GDNF, PTN or their combination were able to show an increase in axon number. Axon diameter and re-myelination were only moderately influenced by the PTN-GDNF treatment, further suggesting a limitation in the growth-promoting milieu provided by the distal stump to the regenerating axons.

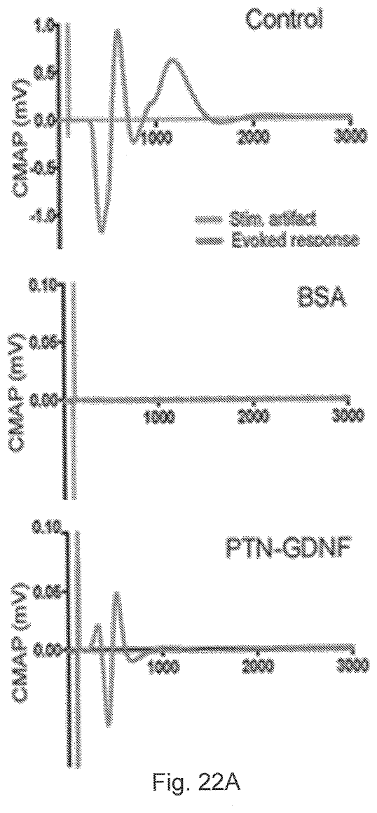

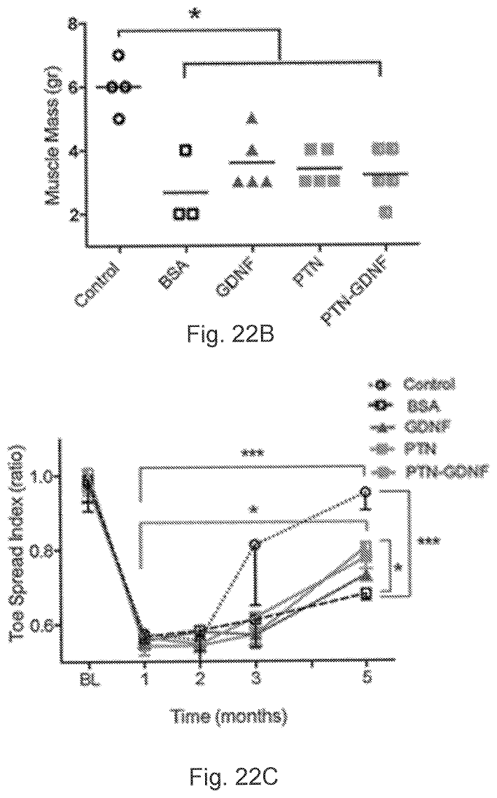

The electrophysiological evaluation of compound motor action potentials on the tibialis anterior muscle in the injured leg five months after implantation, confirmed that electrical stimulation of the nerve proximal to the repair site, evoked an action potential peak (0.70±0.20 mV) at 3× threshold in the cut-resuture group (FIG. 22A). Conversely, no CMAP activity was evoked in animals implanted with BSA, GDNF or PTN (FIG. 22B). Only 1 of 5 rabbits repaired with PTN-GDNF showed evoked CMAP, with only 10%(0.05 mV) of the amplitude compared to cut-resuture controls (FIG. 22C).

Evaluation of the recovery of function by toe-spread index, assessed during five months after the 4 cm gap repair, revealed that the ability for toe extension dropped 40% immediately after injury (Sup. 2 C) as compared to baseline (Sup.2 B) and improved over the course of 5 months (Sup.2 D). Signs of functional recovery were first observed in the cut-resuture group at 3 months after injury, whereas those with BNI showed signs of improving two months later. At month 5, animals implanted with PTN-GDNF were significantly different compared to those with BSA, and showed a 76% improvement compared to the estimated 95% function of the cut-resuture control group. Additionally, we tested the ability of all animals to sense an irritant (formalin) on the dorsal part of their foot. Because of the caustic nature of this test, it was performed only once, at 5 months after injury. The animals that did not respond to the injections were removed from the analysis. Although there was no statistical difference between all groups, the data indicated that the BSA group had the highest lick count suggesting hyperalgesia (Sup. 3 A-B). Finally, the tibialis anterior muscle mass was measured and was significantly larger in the cut-resuture control (6±0.41 g), compared to that in BNI implanted animals with growth factors (3.2-3.6±0.4 g) and those treated with BSA, which were not significantly different form one another (2.67±0.7 g) (FIG. 22). Together, our findings demonstrated that the topographical multiluminal design of the BNI with the co-administration of PTN-GDNF allowed the regeneration of axons across a challenging 4 cm critical gap and restoration of partial function after common peroneal nerve lesion in rabbits.

### D. Discussion

This Example used multi-luminal BNIs filled with growth factor-encapsulated PLGA microparticles suspended in collagen type I to test the effect of pleiotrophins, glial derived growth factors and neurotrophins on the functional repair of 4 cm critical injury gap. It was first confirmed that PTN was able to entice nerve growth across a 3 cm nerve gap without inducing abnormal growth. The relative potency of single and combined growth factors was evaluated in stimulating the regeneration of sensory and motor neurons in vitro. The most effective treatment was PTN-GDNF. It was confirmed in the rabbit common peroneal model that this neurotrophin-pleiotrophin combination is effective in mediating nerve regeneration across a 4 cm critical injury compared to those with BSA, GDNF and PTN alone indicated by the gross anatomy and histology. The observed nerve regeneration 2 mm distal to the implant and partial behavioral recovery supports the notion that sustained release of growth factors with broad cellular target range, including Schwann cells, macrophages, fibroblasts, and endothelial cells, provides a more effective regenerative strategy compared to hollow conduits.

While several combinations of growth factors have shown to be synergistic, the Example herein demonstrates that the combination of PTN with GDNF results in the highest number of responding neurons and the longest axon length in vitro. Not intending to be bound by theory, it is believed that GDNF and PTN can be synergistic not only by activating different cell types in the injured nerve, but also by potentiating the response of individual cells. In summary, the results of this Example demonstrate that the topographical multiluminal structural design of the BNI with sustained PTN-GDNF release successfully entice functional nerve regeneration across a 4 cm long gap defect.

### E. Materials and Methods

### 1. Dorsal Root Ganglia and Spinal Cord Explant Cultures

Neonatal (P0-P4) dorsal root ganglia (DRG) and spinal cord were dissected from CD1 mice as previously described⁴⁸. The DRG explants and SC sections were placed onto poly-D-lysine (PDL) coated wells, immobilized with collagen (85% type I, 15% type II; Millipore; Temecula, Calif.), and incubated at 37° C. with 5% CO₂ in neurobasal-A media (Sigma Aldrich; St. Louis, Mo.) supplemented with 2% B27, 0.5% penicillin/streptomycin, and 0.75% L-glutamine. The media was then supplemented with one or more of the following molecules: a) neurotrophins: BDNF, NGF or/and NT3, b) Glial-derived: GDNF, and c) pleiotrophins: PTN or/and VEGF (5-100 ng/mLPeproTech Inc., Rock Hill, NJ). Growth factors were added individually or in combination 24 hours after plating. The controls received media without growth factors. After 3 days in vitro, the explants were fixed in 4% paraformaldehyde (PFA). Using standard immunohistochemistry techniques, the DRG were labeled with mouse anti-β tubulin III antibody (1:400; Sigma Aldrich, St. Louis, Mo.) and the SC explants with mouse anti RT97 antibody (1: 200, Santa Cruz Biotech, Dallas, Tex.) to label the neurites from sensory and motor neurons, respectively. Axonal length and density were visualized using 10× objectives on a Zeiss confocal microscope (Zeiss Axioplan 2 LSM 510 META) for both DRG explants and spinal cord sections. Axonal length was measured from the ganglia to the most distal end of the axons using Axiovision LM software (CarlZeiss, Axiocam version 4.2.0.1). Axonal density was estimated using optical densitometry and analyzed using the histogram selection in an average of 50 axons per explant in three different experiments performed in triplicate (n=9).

2. Biosynthetic Nerve Implants

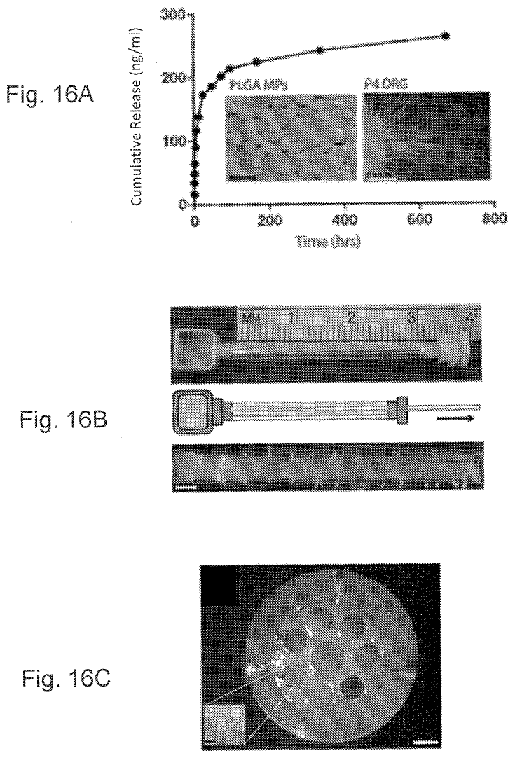

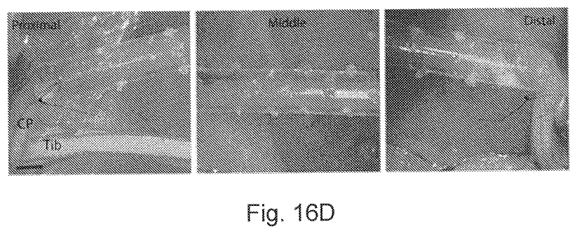

The biosynthetic nerve implant (BNI) conduits consisted of a transparent polyurethane conduit (Micro-Renathane®; Braintree Scientific, Inc., OD 3 mm, ID 1.75 mm and length of 3 or 4 cm) with 8 microchannels casted in the lumen using 1.5% agarose. Each micro-channel was filled with type I collagen mixed with either BSA, PTN, VEGF, GDNF, or PTN-GDNF (DL-lactic-co-glycolic acid) encapsulated PLGA microparticles (MPs; FIG. 16A). The MPs were prepared using double emulsion as previously described⁴⁹. We added 10 μg/mL of BSA (Sigma-Aldrich, St Louis, Mo.), 20 μg/μL of PTN, 5 μg/μL of VEGF (Invitrogen, Carlsbad, Calif.), or 20 μg/μL of GDNF (PeproTech Inc., Rocky Hill, N.J.) to 50:50 PLGA, sonicated and lyophilized. The MP size was determined by scanning electron microscopy (SEM, Hitachi S-3000 N Variable Pressure) and zeta potential size analyzer. The average size of particles were measured to be 1.2±0.06 μm in diameter. PTN particles release showed an initial burst within the first 24 hours followed by a sustained release of 85% over 28 days using ELISA assay (TSZ ELISA, HU9951). The bioactivity of the encapsulated protein was evaluated in vitro by determining the axonal growth of DRG. The average axonal length was significantly higher when treated with PTN MPs (744.6±133 μm) in comparison to the negative control (542.4±166.9 μm) and BSA (552.7±156.5 μm) treated groups (FIG. 16B). Casting the microchannels and loading the growth factor MPs was done simultaneously as described⁵⁰. Briefly, a custom made casting scaffold was used to guide metal fibers in the lumen of the conduit and melted agarose was injected into the lumen covering the metal fibers. After polymerization, the collagen-MPs was loaded into a chamber in which the fibers were previously inserted. Pulling the fibers out casted the microchannels and filled the lumen with the collagen-MPs (FIG. 16C-D).

### 3. Animal Surgery

A total of 49 adult New Zealand white rabbits (Myrtle Rabbitry, Tenn.) weighing between 1.5 to 1.8 kilograms were used in this study. The first cohort of 25 animals were used to evaluate the effect of BNI-VEGF (n=7) and BNI-PTN (n=7) in a 3 cm gap compared to BNI-collagen (n=7) or collagen-filled nerve guides (n=4; negative controls). Two rabbits from each group were used for the H&E staining. A second cohort of 24 animals were used to compare the effect of BNIs filled with either GDNF (n=6), PTN (n=6) or a combination of PTN-GDNF (n=5) compared to BNI-BSA (n=3; negative control) and cut-resuture (n=4; positive control) in a 4 cm critical gap. The peroneal nerve was exposed through a muscle-sparing incision along the sciatic vein between the semitendinosus and the biceps muscles. The two muscles were gently spread to expose the proximal part of the undivided peroneal nerve. A 20-40 mm segment was removed, and the proximal and distal nerve stumps were sutured to the conduit or BNI (either 3 or 4 cm long) and secured it to the underlying muscle with absorbable sutures (FIG. 16). The muscle was sutured and the skin stapled. Topical antibiotic ointment was applied prophylactically. All animals received antibiotic (trimethoprim-sulfa; 0.5 mg/kg oral) and analgesic (sustained release Buprenorphine; 1 mg/kg, SC) treatments post-surgery. The surgeon and researchers were blinded to the BNI treatments.

All animal procedures were performed in accordance with the guidelines of the Institutional Animal Care and Use Committee of the University of Texas at Arlington according to the NIH guide for the care and use of laboratory animals.

4. Toe spread index

The toe-spread index (TSI) was used to evaluate the deep peroneal nerve reinnervation to the tibialis anterior, the extensor hallucis longus, the extensor digitorum longus, and the fibularis tertius muscles located in the anterior compartment of the leg. TSI is a sensitive indicator of the onset of motor recovery after peroneal injury where the animal will spread their toes reflexively in an attempt to maximize the surface area of the foot for a safer landing⁵¹. The rabbits were held by the loose skin on their back and suddenly lowered a few times to evoke a startle response characterized by toe extension. For the 4cm gap study, the toe spread index was measured using a custom designed apparatus (Sup. 2A) to ensure stability and to eliminate extraneous variables related to human imprecision. The rabbit was secured to the hook via a harness placed at the top of a mobile metal element which itself was attached to the wooden base. By removing the metal element, the rabbit will fall freely and come to a halt after a specific distance. Multiple frames from video recordings were selected to measure the distance between the first and the fourth toes during the startle response using the ImageJ software. Measurements were taken prior to surgery and monthly for the duration of the study. The toenails were colored to facilitate their visualization. The index was calculated as a ratio of the toe-spread of the healthy foot to that of the injured foot (Sup. 2B-D).

### 5. Formalin Test

The animals were tested for sensory reinnervation by evaluating the “itch” response to formalin test. A 0.1 ml subcutaneous injection of 2% formalin in PBS on the dorsal part of the injured foot between the two inner most toes, approximately 1 cm in the dorsal anterior portion of the foot over the common peroneal sensory dermatome was administered. The injection causes skin irritation, that when perceived by the animal, results in a licking behavior on the injected area. The total number of “licks” were recorded.

### 6. Electrophysiological Evaluation

Five months after implantation, the common peroneal nerves of the left (injured) and right (uninjured) legs were re-exposed under anesthesia. Bipolar hook electrodes were placed proximal to the BNI and constant voltage biphasic pulses were applied using the AM systems Isolated Stimulator. The motor recruitment from the tibialis anterior muscle was recorded using a second set of bipolar needle electrodes via the Biopack MP36 system. Signal amplitudes for the treatment groups were compared to the cut-resuture control to evaluate strength of evoked responses.

7. Immunohistochemistry Following perfusion, the BNI conduits, the cut-resuture control and the healthy nerve were carefully dissected and the tissue was post-fixed in 4% PFA. The polyurethane tubes were separated from the regenerated nerve tissue and divided into 3 different regions, proximal, middle and distal and embedded in paraffin. Ten micron sections were labeled using antibodies for NFP, β tubulin III (1:400; Sigma Aldrich) and P0 (1:200; Millipore) overnight at 4° C. The tissue was then incubated for 1 hour with Cy2-and Cy3 conjugated secondary antibodies (1:400; Sigma Aldrich, St. Louis, Mo.). The slides were counterstained and coverslipped. A Zeiss confocal microscope (Zeiss Axioplan 2 LSM 510 META) was used to evaluate the tissue.

### 8. Electron Microscopy

A nerve segment 2.5 mm distal to the repair site was post-fixed in 2.5% glutaraldehyde in 0.1 M cacodylate buffer. The tissue was then embedded in epoxy resin and 1 μm thin sections cut and stained with osmium and examined at 6000× magnification using a JEOL LEM 1200 EX II system. Ten regions across each sample were randomly selected for quantifying axon number, axon diameter, and g-ratio using the ImageJ software. 9. Statistical Analysis

Analysis of Variance was used to test for statistical significance followed by post hoc Tukey's or Dunn's multiple comparison tests using Prism 7 software (GraphPad, Inc). Data collected from the study are represented as the average of the mean±standard deviation unless otherwise noted. P values<0.05 were considered significant.

### F. References

1 Hoke, A. & Brushart, T. Introduction to special issue: Challenges and opportunities for regeneration in the peripheral nervous system. Exp Neurol 223, 1-4, doi:10.1016/j.expneurol.2009.12.001 (2010).
2 Grinsell, D. & Keating, C. P. Peripheral nerve reconstruction after injury: a review of clinical and experimental therapies. Biomed Res Int 2014, 698256, doi:10.1155/2014/698256 (2014).
3 Fu, S. Y. & Gordon, T. Contributing factors to poor functional recovery after delayed nerve repair: prolonged axotomy. J Neurosci 15, 3876-3885 (1995).
4 Krarup, C., Archibald, S. J. & Madison, R. D. Factors that influence peripheral nerve regeneration: an electrophysiological study of the monkey median nerve. Ann Neurol 51, 69-81 (2002).
5 Moore, A. M. et al. Limitations of conduits in peripheral nerve repairs. Hand (NY) 4, 180-186, doi:10.1007/s11552-008-9158-3 (2009).
6 Pabari, A., Lloyd-Hughes, H., Seifalian, A. M. & Mosahebi, A. Nerve conduits for peripheral nerve surgery. Plast Reconstr Surg 133, 1420-1430, doi:10.1097/PRS.0000000000000226 (2014).
7 Gordon, T., Tyreman, N. & Raji, M. A. The basis for diminished functional recovery after delayed peripheral nerve repair. J Neurosci 31, 5325-5334, doi:10.1523/JNEUROSCI.6156-10.2011 (2011).
8 Hoke, A. et al. Schwann cells express motor and sensory phenotypes that regulate axon regeneration. J Neurosci 26, 9646-9655, doi:10.1523/JNEUROSCI.1620-06.2006 (2006).
9 Brushart, T. M. et al. Schwann cell phenotype is regulated by axon modality and central-peripheral location, and persists in vitro. Exp Neurol 247, 272-281, doi:10.1016/j.expneurol.2013 .05.007 (2013).
10 Romero, M. I., Rangappa, N., Garry, M. G. & Smith, G. M. Functional regeneration of chronically injured sensory afferents into adult spinal cord after neurotrophin gene therapy. The Journal of neuroscience: the official journal of the Society for Neuroscience 21, 8408-8416, doi:21/21/8408 [pii] (2001).
11 Gordon, T. The role of neurotrophic factors in nerve regeneration. Neurosurg Focus 26, E3, doi:10.3171/FOC.2009.26.2.E3 10.3171/FOC.2009.26.2.E3 [pii] (2009).
12 Romero, M. I. et al. Deletion of Nf1 in neurons induces increased axon collateral branching after dorsal root injury. The Journal of neuroscience: the official journal of the Society for Neuroscience 27, 224-2134, doi:27/8/2124 [pii] 10.1523/JNEUROSCI.4363-06.2007 (2007).
13 Patel, M., Mao, L., Wu, B. & Vandevord, P. J. GDNF-chitosan blended nerve guides: a functional study. J Tissue Eng Regen Med 1, 360-367, doi:10.1002/term.44 (2007).
14 Wood, M. D. et al. GDNF released from microspheres enhances nerve regeneration after delayed repair. Muscle Nerve 46, 122-124, doi:10.1002/mus.23295 (2012).
15 Tajdaran, K., Gordon, T., Wood, M. D., Shoichet, M. S. & Borschel, G. H. A glial cell line-derived neurotrophic factor delivery system enhances nerve regeneration across acellular nerve allografts. Acta Biomater 29, 62-70, doi:10.1016/j.actbio.2015.10.001 (2016).
16 Boyd, J. G. & Gordon, T. Glial cell line-derived neurotrophic factor and brain-derived neurotrophic factor sustain the axonal regeneration of chronically axotomized motoneurons in vivo. Exp Neurol 183, 610-619 (2003).
17 Magill, C. K. et al. The differential effects of pathway—versus target-derived glial cell line-derived neurotrophic factor on peripheral nerve regeneration. J Neurosurg, doi:10.3171/2009.10.JNS091092 (2009).
18 Madduri, S., Feldman, K., Tervoort, T., Papaloizos, M. & Gander, B. Collagen nerve conduits releasing the neurotrophic factors GDNF and NGF. J Control Release, doi: S0168-3659(09)00862-1 [pii] 10.1016/j.jconre1.2009.12.017 (2009).
19 Fang, W., Hartmann, N., Chow, D. T., Riegel, A. T. & Wellstein, A. Pleiotrophin stimulates fibroblasts and endothelial and epithelial cells and is expressed in human cancer. J Biol Chem 267, 25889-25897 (1992).
20 Blondet, B., Carpentier, G., Lafdil, F. & Courty, J. Pleiotrophin cellular localization in nerve regeneration after peripheral nerve injury. J Histochem Cytochem 53, 971-977, doi:10.1369/jhc.4A6574.2005 (2005).
21 Xu, C., Zhu, S., Wu, M., Han, W. & Yu, Y. Functional receptors and intracellular signal pathways of midkine (MK) and pleiotrophin (PTenn.). Biol Pharm Bull 37, 511-520 (2014).
22 Kadomatsu, K. & Muramatsu, T. Midkine and pleiotrophin in neural development and cancer. Cancer Lett 204, 127-143, doi:10.1016/S0304-3835(03)00450-6 (2004).
23 Ochiai, K., Muramatsu, H., Yamamoto, S., Ando, H. & Muramatsu, T. The role of midkine and pleiotrophin in liver regeneration. Liver Int 24, 484-491, doi:10.1111/j.1478-3231.2004.0990.x (2004).
24 Blondet, B., Carpentier, G., Ferry, A. & Courty, J. Exogenous pleiotrophin applied to lesioned nerve impairs muscle reinnervation. Neurochem Res 31, 907-913, doi:10.1007/s11064-006-9095-x (2006).
25 Mi, R., Chen, W. & Hoke, A. Pleiotrophin is a neurotrophic factor for spinal motor neurons. Proc Natl Acad Sci U S A 104, 4664-4669, doi:0603243104 [pii] 10.1073/pnas.0603243104 (2007).
26 Santos, D., Giudetti, G., Micera, S., Navarro, X. & del Valle, J. Focal release of neurotrophic factors by biodegradable microspheres enhance motor and sensory axonal regeneration in vitro and in vivo. Brain Res 1636, 93-106, doi:10.1016/j.brainres.2016.01.051 (2016).
27 Madduri, S., di Summa, P., Papaloizos, M., Kalbermatten, D. & Gander, B. Effect of controlled co-delivery of synergistic neurotrophic factors on early nerve regeneration in rats. Biomaterials 31, 8402-8409, doi:10.1016/j.biomaterials.2010.07.052 (2010).
28 de Boer, R. et al. Short- and long-term peripheral nerve regeneration using a poly-lactic-co-glycolic-acid scaffold containing nerve growth factor and glial cell line-derived neurotrophic factor releasing microspheres. J Biomed Mater Res A 100, 2139-2146, doi:10.1002/jbm.a.34088 (2012).
29 Stoica, G. E. et al. Midkine binds to anaplastic lymphoma kinase (ALK) and acts as a growth factor for different cell types. J Biol Chem 277, 35990-35998, doi:10.1074/jbc.M205749200 (2002).
30 Muramatsu, T. Midkine and pleiotrophin: two related proteins involved in development, survival, inflammation and tumorigenesis. J Biochem 132, 359-371 (2002).
31 Degoutin, J., Brunet-de Carvalho, N., Cifuentes-Diaz, C. & Vigny, M. ALK (Anaplastic Lymphoma Kinase) expression in DRG neurons and its involvement in neuron-Schwann cells interaction. Eur J Neurosci 29, 275-286, doi:10.1111/j.1460-9568.2008.06593.x (2009).
32 Bonanomi, D. et al. Ret is a multifunctional coreceptor that integrates diffusible- and contact-axon guidance signals. Cell 148, 568-582, doi:10.1016/j.ce11.2012.01.024 (2012).
33 Baudet, C. etal. Positive and negative interactions of GDNF, NTN and ART in developing sensory neuron subpopulations, and their collaboration with neurotrophins. Development 127, 4335-4344 (2000).
34 Luo, W. et al. A hierarchical NGF signaling cascade controls Ret-dependent and Ret-independent events during development of nonpeptidergic DRG neurons. Neuron 54, 739-754, doi:10.1016/j.neuron.2007.04.027 (2007).
35 Fontana, X. et al. c-Jun in Schwann cells promotes axonal regeneration and motoneuron survival via paracrine signaling. J Cell Biol 198, 127-141, doi:10.1083/jcb.201205025 (2012).
36 Paveliev, M. et al. Sensory neurons from N-syndecan-deficient mice are defective in survival. Neuroreport 19, 1397-1400, doi:10.1097/WNR.0b013e32830d1486 (2008).
37 Murakami, K. & Yoshida, S. Nerve injury induces the expression of syndecan-1 heparan sulfate proteoglycan in peripheral motor neurons. Neurosci Lett 527, 28-33, doi:10.1016/j.neulet.2012.08.043 (2012).
38 Murakami, K., Tanaka, T., Bando, Y. & Yoshida, S. Nerve injury induces the expression of syndecan-1 heparan sulfate proteoglycan in primary sensory neurons. Neuroscience 300, 338-350, doi:10.1016/j.neuroscience.2015.05.033 (2015).
39 Meng, K. etal. Pleiotrophin signals increased tyrosine phosphorylation of beta beta-catenin through inactivation of the intrinsic catalytic activity of the receptor-type protein tyrosine phosphatase beta/zeta. Proc Natl Acad Sci U S A 97, 2603-2608, doi:10.1073/pnas.020487997 (2000).
40 Tchetchelnitski, V., van den Eijnden, M., Schmidt, F. & Stoker, A. W. Developmental co-expression and functional redundancy of tyrosine phosphatases with neurotrophin receptors in developing sensory neurons. Int J Dev Neurosci 34, 48-59, doi:10.1016/j.ijdevneu.2014.01.005 (2014).
41 Harroch, S. et al. A critical role for the protein tyrosine phosphatase receptor type Z in functional recovery from demyelinating lesions. Nat Genet 32, 411-414, doi:10.1038/ng1004 (2002).
42 Pariser, H., Ezquerra, L., Herradon, G., Perez-Pinera, P. & Deuel, T. F. Fyn is a downstream target of the pleiotrophin/receptor protein tyrosine phosphatase beta/zeta-signaling pathway: regulation of tyrosine phosphorylation of Fyn by pleiotrophin. Biochem Biophys Res Commun 332, 664-669, doi:10.1016/j.bbrc.2005.05.007 (2005).
43 Fujikawa, A. & Noda, M. Role of pleiotrophin-protein tyrosine phosphatase receptor type Z signaling in myelination. Neural Regen Res 11, 549-551, doi:10.4103/1673-5374.180761 (2016).
44 Feltri, M. L., Poitelon, Y. & Previtali, S. C. How Schwann Cells Sort Axons: New Concepts. Neuroscientist 22, 252-265, doi:10.1177/1073858415572361 (2016).
45 Shin, Y. K. et al. Grb2-associated binder-1 is required for neuregulin-l-induced peripheral nerve myelination. J Neurosci 34, 7657-7662, doi:10.1523/JNEUROSCI.4947-13.2014 (2014).
46 Fricker, F. R. et al. Axonally derived neuregulin-1 is required for remyelination and regeneration after nerve injury in adulthood. J Neurosci 31, 3225-3233, doi:10.1523/JNEUROSCI.2568-10.2011 (2011).
47 Stassart, R. M. et al. A role for Schwann cell-derived neuregulin-1 in remyelination. Nat Neurosci 16, 48-54, doi:10.1038/nn.3281 (2013).
48 Alsmadi, N. Z. et al. Coiled polymeric growth factor gradients for multi-luminal neural chemotaxis. Brain Res 1619, 72-83, doi:10.1016/j.brainres.2015.01.055 (2015).
49 Dawood, A. L., P.; Dash SN.; Kona, SK.; Nguyen, KT.; Romero-Ortega, MI. EGF Release in Multiluminal Hydrogels Directs Angiogenesis from Adult Vasculature In Vitro. Cardiovascular Engineering and Technology 2, 173 (2011).
50 Tansey, K. E., Seifert, J. L., Botterman, B., Delgado, M. R. & Romero, M. I. Peripheral nerve repair through multi-luminal biosynthetic implants. Annals of biomedical engineering 39, 1815-1828, doi:10.1007/s10439-011-0277-6 (2011).
51 Schmitz, H. C. & Beer, G. M. The toe-spreading reflex of the rabbit revisited—functional evaluation of complete peroneal nerve lesions. Lab Anim 35, 340-345, doi:10.1258/0023677011911930 (2001).

### EXAMPLE 4

### Myelination of Nerves

In the present non-limiting Example it is demonstrated that neuregulin 1 type III (NRG1-III) promotes re-myelination in long gap nerve injuries.

Neuregulin 1 (NRG1) a signaling molecule that belongs to an epidermal growth factor like family, also known as GGF; HGL; HRG; NDF; ARIA; GGF2; HRG1; HRGA; SMDF; MST131; MSTP131; NRG1-IT2. This molecule is membrane glycoprotein that mediates cell-cell signaling and plays a critical role in the growth and development of multiple organ systems by binding to tyrosine kinase ErbB receptors. It has been involved in neurite outgrowth, adhesion, apoptosis, neuron migration, and differentiation of glial cells including astrocyte, oligodendrocytes, and Schwann cells. A large variety of different isoforms are produced from this gene through alternative promoter in a tissue-specific manner, classified as types I, II, III, IV, V and VI.

Of these, NRG1 Type II has been implicated in Schwann cell maturation and myelination of peripheral nerves, during development and after injury, and NRG1 Type III promotes the conversion of pre-cursor Schwann cells into pro-myelinating or Remak type. It also regulates the ensheathment of axons and myelin sheath thickness After injury, this protein signals the Schwann cells to incorporate laminin along the regenerative bridge facilitating axonal growth.

Previous Examples report the use of glial-derived neurotrophic factor (GDNF) and pleiotrophin (PTenn.) stimulated the successful regeneration of the peripheral nerve across a 40 mm nerve gap. Although the combination of these growth factors resulted in maximum functional recovery and highest number of axons as compared to the negative control, there was minimal re-myelination, which is required to regain function of motor and some sensory neurons. Electron microscopy showed the regenerated nerves did not re-myelinate due to abnormal axonal sorting, as large axons were arrested within a Remak bundles. Axonal sorting and subsequent re-myelination depends on the expression of NRG1 type-III by the regenerated axons. NRG1-type-III also known as sensory and motor neuron-derived factor (SMDF). This is a pro-neuregulin-1 that remains tethered to the axonal membrane through a hydrophobic N-terminal cysteine-rich domain (CRD). This was done as it is known that truncated NRG1 isoforms in tissue extracts has suggested that proteolytic processing of full-length NRG1 isoforms is needed for its activity. The fragment used in the present example contained a C-terminal EGF-like domain (β-variant) and a unique N terminal sequence that lacks an Ig-like domain and the transmembrane domain and the cytoplasmic tail, and was used to stimulate Schwann cells to make myelin in nerves that were enticed to regenerated across a long gap injury by the use of growth factors.

### 1. Encapsulation

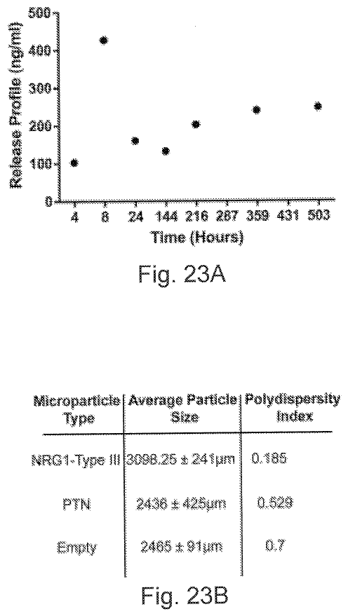

Recombinant Human NRG1 Isoform SMDF Protein was encapsulated to test if this molecule combined with PTN would allow escape of axons from the Remak bundle and promote remyelination. NRG1-type III was encapsulated in polylactic-co-glycolic acid (PLGA) microspheres as described before. These microspheres provided a sustained released of the protein over a period of twenty eight days, and its bioactivity was confirmed in vitro using dorsal root ganglia (DRG) explants (FIG. 23).

2. Effective Nerve Repair with Re-Myelination Over 4 cm Gap

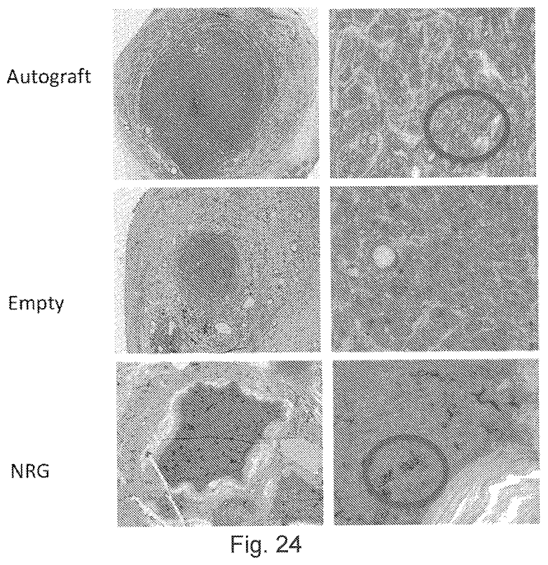

The microspheres were then loaded in the Biosynthetic Nerve Implant (BNI), previously described, and used to repair 40 mm gap in the peroneal nerve of rabbits. After 26 weeks of recovery, the nerve was dissected and evaluated for the quality of nerve regeneration. FIG. 24 shows examples of regenerated nerves and qualitatively showed better nerve regeneration in the NRG and NRG-PTN groups compared to those with empty particles. Histological staining of the tissue in the conduits confirmed the presence of axons and, most importantly, re-myelination was confirmed for the first time in these long gap regenerated nerves (FIG. 24).

4. Increased Recovery of Function by Combined Growth Factor (PTenn.) with a Re-Myelination Promoting Molecule (NRG1-III).

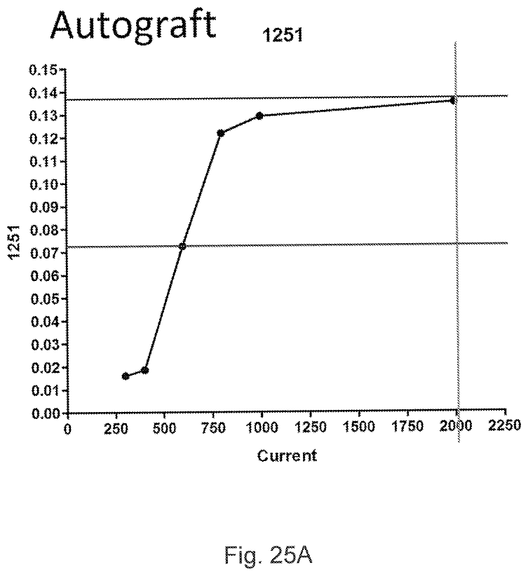

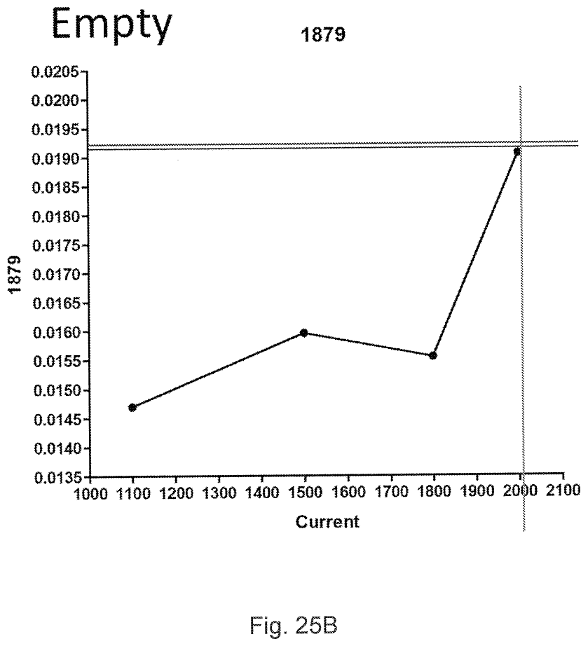

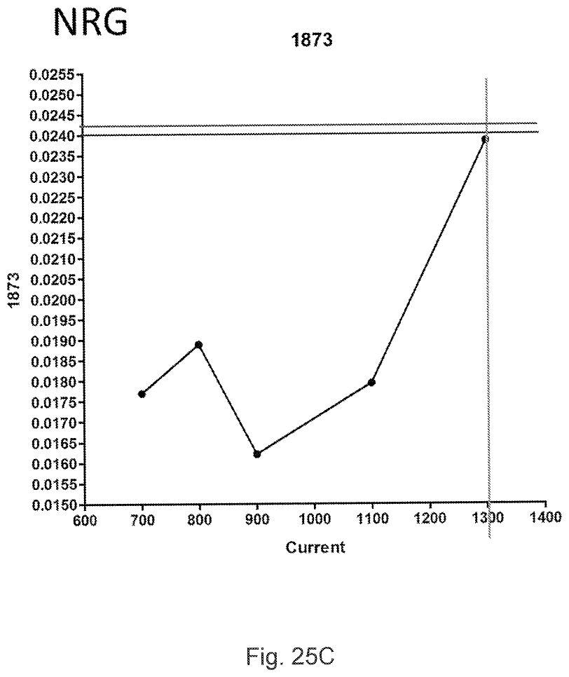

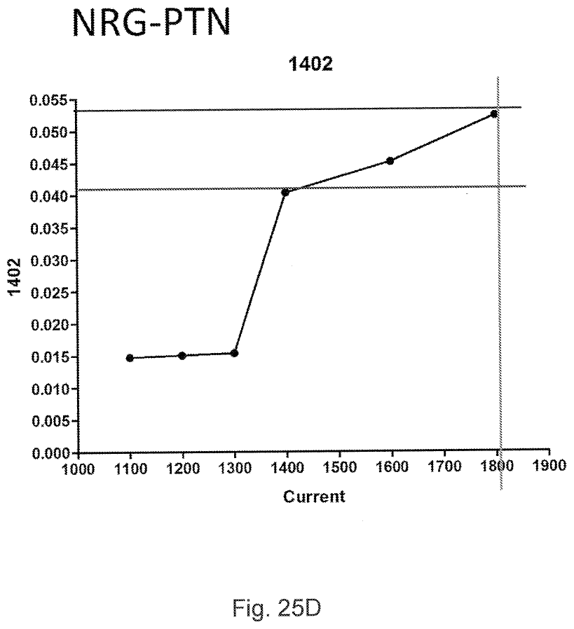

The functionality of the regenerated nerve was tested by electrically stimulating it before the injury site and recording the evoked compound action potential. Compared to the autograft that responded with 500 μA with a 0.07 μV signal (threshold), the BNI with no growth factors needed 2000 μA and evoked only a 0.02 μV signal. Adding NRG1-III decreased the threshold to 1300 μA compared to the empty microparticles but only evoked a 0.024 μV signal. In contrast, the combination of both PTN and NRG1-III showed a 1400 μA with a signal of 0.04 μV (FIG. 25).

In summary, nerve regeneration is composed of at least two processes: one is the regeneration of the axons across the injury gap, and second is the re-myelination of the regenerating axons. This last step is critical for enable “fast conduction” which is require for motor recovery and some large sensory neurons. This Example illustrates the second requirement.

Additional exemplary embodiments are provided below.
Embodiment 1. A method of promoting asymmetric nerve growth, the method comprising:
   exposing a population of transected nerves to a first molecular growth cue and to a second molecular growth cue,
   wherein the population of transected nerves comprises one or more nerves of a first nerve type and one or more nerves of a second nerve type differing from the first nerve type;
   wherein the first molecular growth cue preferentially promotes growth of the first nerve type, as compared to the second nerve type;
   wherein the second molecular growth cue preferentially promotes growth of the second nerve type, as compared to the first nerve type; and
   wherein the first molecular growth cue is spatially separated from the second molecular growth cue.
Embodiment 2. The method of embodiment 1, wherein:
   nerves of the first nerve type preferentially grow toward a first spatial region comprising the first molecular growth cue; and
   nerves of the second nerve type preferentially grow toward a second spatial region comprising the second molecular growth cue.
Embodiment 3. The method of embodiment 2, wherein the first spatial region is defined by a first lumen and the second spatial region is defined by a second lumen differing from the first lumen.
Embodiment 4. The method of any preceding embodiments, wherein:
   the nerves of the first nerve type are motor nerves; and
   the nerves of the second nerve type are sensory nerves or autonomic nerves.
Embodiment 5. The method of any preceding embodiment, wherein:
   the population of transected nerves comprises peripheral nerves.
Embodiment 6. The method of any of the preceding embodiment, wherein:
   the population of transected nerves comprises axons from neurons in the central nervous system.
Embodiment 7. The method of any preceding embodiment, wherein:
   the population of transected nerves comprises somatic nerves or autonomic nerves.
Embodiment 8. The method of any preceding embodiment, wherein:
   the first molecular growth cue comprises an attractive molecular growth cue for the first nerve type; and
   the second molecular growth cue comprises an attractive molecular growth cue for the second nerve type.
Embodiment 9. The method of any preceding embodiment, wherein:
   the first molecular growth cue comprises an attractive molecular growth cue for the first nerve type; and
   the second molecular growth cue comprises a repulsive molecular growth cue for the first nerve type.
Embodiment 10. The method of any preceding embodiment, wherein:
   the first molecular growth cue comprises a repulsive molecular growth cue for the second nerve type; and
   the second molecular growth cue comprises a repulsive molecular growth cue for the first nerve type.
Embodiment 11. The method of any preceding embodiment, wherein:
   the first molecular growth cue comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).
Embodiment 12. The method of any preceding embodiment, wherein:
   the second molecular growth cue comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).
Embodiment 13. The method of any preceding embodiment, wherein:
   the first molecular growth cue and/or the second molecular growth cue comprises a netrin, Slit protein, ephrin, semaphorin, cell adhesion molecule, or a combination of two or more of the foregoing.
Embodiment 14. The method of embodiment 13, wherein the first molecular growth cue or the second molecular growth cue comprises Semaphorin 3A.
Embodiment 15. The method of embodiment 4, wherein:
   the first molecular growth cue comprises GDNF; and
   the second molecular growth cue comprises PTN.
Embodiment 16. The method of any preceding embodiment further comprising exposing the nerves of the first nerve type and/or the nerves of the second nerve type to a third molecular growth cue.
Embodiment 17. The method of embodiment 16, wherein the third molecular growth cue promotes myelination of the nerves of the first nerve type and/or the nerves of the second nerve type.
Embodiment 18. The method of embodiment 16 or 17, wherein the third molecular growth cue stimulates Schwann cells.
Embodiment 19. The method of any one of embodiments 16-18, wherein the third molecular growth cue is provided in microparticles.
Embodiment 20. The method of embodiment 19, wherein the microparticles have an average diameter between 1500 and 3500 μm.
Embodiment 21. The method of any one of embodiments 16-20, wherein the step of exposing the nerves of the first nerve type and/or the nerves of the second nerve type to a third molecular growth cue comprises using a sustained-release profile for the third molecular growth cue for at least 20 days.
Embodiment 22. The method of any one of embodiments 16-21, wherein the third molecular growth cue comprises a neuregulin (NRG).
Embodiment 23. The method of embodiment 22, wherein the third molecular growth cue comprises neuregulin 1 type III (NRG1-III).
Embodiment 24. A device for promoting asymmetric nerve growth, the device comprising:
   a lumen having a proximal end and a distal end; and
   a matrix material disposed in the lumen;
   wherein the proximal end of the lumen comprises a proximal opening operable to receive nerve tissue;
   wherein the distal end of the lumen comprises a distal opening operable to receive nerve tissue;
   wherein the matrix material defines one or more first microchannels and one or more second microchannels extending from the proximal end of the lumen toward the distal end of the lumen, the first microchannels differing from the second microchannels;
   wherein a first molecular growth cue is disposed within the first microchannels;
   wherein a second molecular growth cue is disposed within the second microchannels, the first molecular growth cue differing from the second molecular growth;
   wherein the first molecular growth cue preferentially promotes growth of a first nerve type, as compared to a second nerve type differing from the first nerve type; and
   wherein the second molecular growth cue preferentially promotes growth of the second nerve type, as compared to the first nerve type.
Embodiment 25. The device of embodiment 24, wherein the distal end of the lumen is bifurcated into a first branch and a second branch.
Embodiment 26. The device of embodiment 25, wherein the first microchannels are disposed in the first branch and the second microchannels are disposed in the second branch.
Embodiment 27. The device of any one of embodiments 24-26, wherein:
   the first molecular growth cue comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).
Embodiment 28. The device of any one of embodiments 24-27, wherein:
   the second molecular growth cue comprises one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), and persephin (PSPN).
Embodiment 29. The device of any one of embodiments 24-28, wherein:
   the first molecular growth cue comprises GDNF; and
   the second molecular growth cue comprises PTN.
Embodiment 30. The device of any one of embodiments 24-29, wherein:
   the first molecular growth cue comprises GDNF; and
   the second molecular growth cue comprises BDNF or PTN and Sema3A.
Embodiment 31. The device of any one of embodiments 24-29, wherein a third molecular growth cue is disposed within the first microchannels and/or second microchannels.
Embodiment 32. The device of embodiment 31, wherein the third molecular growth cue promotes remyelination of the first nerve type and/or the second nerve type.
Embodiment 33. The device of embodiment 31 or 32, wherein the third molecular growth cue comprises a neuregulin (NRG).
Embodiment 34. The device of embodiment 33, wherein the third molecular growth cue comprises neuregulin 1 type III (NRG1-III).
Embodiment 35. A method of regenerating a transected nerve, the method comprising: exposing the transected nerve to a combination of a pleiotrophic growth factor and a neurotrophic growth factor.
Embodiment 36. The method of embodiment 35, wherein the pleiotrophic growth factor comprises PTN.
Embodiment 37. The method of embodiment 35 or 36, wherein the neurotrophic growth factor comprises GDNF.
Embodiment 38. The method of any one of embodiments 35-37, further comprising exposing the transected nerve to a myelination-promoting factor.
Embodiment 39. The method of embodiment 38, wherein the myelination-promoting factor comprises NRG1-III. 0

Embodiment 40. The method of any one of embodiments 35-39 further comprising functionally restoring the transected nerve, wherein the transected nerve is functionally restored by at least 50 percent.
Embodiment 41. A device for regenerating a transected nerve, the device comprising:
   a lumen having a proximal end and a distal end; and
   a matrix material disposed in the lumen;
   wherein the proximal end of the lumen comprises a proximal opening operable to receive nerve tissue;
   wherein the distal end of the lumen comprises a distal opening operable to receive nerve tissue;
   wherein the matrix material defines one or more microchannels extending from the proximal end of the lumen toward the distal end of the lumen;
   wherein a combination of a pleiotrophic growth factor, a myelination-promoting factor, and a neurotrophic growth factor is disposed within the microchannels.
Embodiment 42. A method of promoting nerve myelination, the method comprising: exposing a population of unmyelinated nerves to a myelination-promoting factor.
Embodiment 43. The method of embodiment 42, wherein the exposure is continuous for 1-20 days.
Embodiment 44. The method of embodiment 42 or 43, wherein the unmyelinated nerves are damaged.
Embodiment 45. The method of any one of embodiments 42-44, wherein the unmyelinated nerves are sensory nerves or motor nerves.
Embodiment 46. The method of any one of embodiments 42-45, wherein the unmyelinated nerves are peripheral nerves.
Embodiment 47. The method of any one of embodiments 42-46, wherein the unmyelinated nerves are somatic nerves.
Embodiment 48. The method of any one of embodiments 42-47, wherein the unmyelinated nerves comprises axons from neurons in the central nervous system.
Embodiment 49. The method of any one of embodiments 42-48, wherein the myelination-promoting factor stimulates Schwann cells.
Embodiment 50. The method of any one of embodiments 42-49, wherein the myelination-promoting factor comprises a neuregulin (NRG).
Embodiment 51. The method of embodiment 50, wherein the myelination-promoting factor comprises neuregulin 1 type III (NRG1-III).
Embodiment 52. The method of any one of embodiments 42-51, wherein the myelination-promoting factor is provided in a microparticle.
Embodiment 53. A device for promoting myelination of a nerve, the device comprising:
   a lumen having a proximal end and a distal end; and
   a matrix material disposed in the lumen;
   wherein the proximal end of the lumen comprises a proximal opening operable to receive nerve tissue;
   wherein the distal end of the lumen comprises a distal opening operable to receive nerve tissue;
   wherein the matrix material defines one or more microchannels extending from the proximal end of the lumen toward the distal end of the lumen;
   wherein a myelination-promoting factor is disposed within the microchannels.
Embodiment 54. The method of any one of embodiments 1-23, wherein the first molecular growth cue preferentially stimulates neuronal cells and the second molecular growth cue preferentially stimulates an endothelial cell, a fibroblast, a Schwann cell, a perineural cell, or any combination thereof.
Embodiment 55. The method of any one of embodiments 1-23, wherein the first and/or second and/or third molecular growth cues are provided in microparticles (such as wherein the first molecular growth cue is provided in first microparticles, the second molecular growth cue is provided in second microparticles, and the third molecular growth cue is provided in third microparticles (which first, second, and third microparticles can be formed from the same or different materials and/or have the same or different average sizes compared to one another, in any combination), or when two or all three of the molecular growth cues are provided in the same microparticles).
Embodiment 56. The method of embodiment 55, wherein the microparticles (e.g., the first microparticles or the second microparticles) have an average diameter between 1 and 3500 um.
Embodiment 57. The method of any one of embodiments 1-23, wherein the first molecular growth cue and/or the second molecular growth cue and/or the third molecular growth cue includes a plurality of growth cue species, and the plurality of growth cue species potentiate one another or otherwise provide a synergistic effect (e.g., enhanced nerve growth or enhanced remyelination) as compared to a single species alone.

1. A method of promoting asymmetric nerve growth, the method comprising:
exposing a population of transected nerves to a first molecular growth cue and to a second molecular growth cue,
wherein the population of transected nerves comprises one or more nerves of a first nerve type and one or more nerves of a second nerve type differing from the first nerve type;
wherein the first molecular growth cue preferentially promotes growth of the first nerve type, as compared to the second nerve type;
wherein the second molecular growth cue preferentially promotes growth of the second nerve type, as compared to the first nerve type; and
wherein the first molecular growth cue is spatially separated from the second molecular growth cue.

2. The method of claim 1, wherein:
nerves of the first nerve type preferentially grow toward a first spatial region comprising the first molecular growth cue; and
nerves of the second nerve type preferentially grow toward a second spatial region comprising the second molecular growth cue.

3. The method of claim 2, wherein the first spatial region is defined by a first lumen and the second spatial region is defined by a second lumen differing from the first lumen.

4. The method of claim 1, wherein:
the nerves of the first nerve type are motor nerves; and
the nerves of the second nerve type are sensory nerves.

5. The method of claim 1, wherein:
the population of transected nerves comprises peripheral nerves.

6. The method of claim 1, wherein:
the population of transected nerves comprises axons from neurons in the central nervous system.

7. The method of claim 1, wherein:
the population of transected nerves comprises somatic nerves.

8. The method of claim 1, wherein:
the first molecular growth cue comprises an attractive molecular growth cue for the first nerve type; and
the second molecular growth cue comprises an attractive molecular growth cue for the second nerve type.

9. The method of claim 1, wherein:
the first molecular growth cue comprises an attractive molecular growth cue for the first nerve type; and
the second molecular growth cue comprises a repulsive molecular growth cue for the first nerve type.

10. The method of claim 1, wherein:
the first molecular growth cue comprises a repulsive molecular growth cue for the second nerve type; and
the second molecular growth cue comprises a repulsive molecular growth cue for the first nerve type.

11. The method of claim 1, wherein:
the first molecular growth cue and the second molecular growth cue comprises comprise one or more of neurotrophin-3 (NT-3), neurotrophin-4/5 (NT-4/5), nerve growth factor (NGF), brain-derived neurotrophic factor (BDNF), glial cell derived neurotrophic factor (GNDF), insulin-like growth factors-1/2 (IFG1, IGF2), pleiotrophin (PTenn.), fibroblast growth factor (FGF), vascular endothelial growth factor (VEGF), leukemia inhibitory factor (LIF), ciliary neurotrophic factor (CNTF), interleukin-6 (IL-6), neurturin (NRTenn.), artemin (ARTenn.), persephin (PSPN)_,
wherein the first molecular growth cue and the second molecular growth cue differ from each other.

12. (canceled)

13. The method of claim 1, wherein:
the first molecular growth cue and/or the second molecular growth cue comprises anetrin, Slit protein, ephrin, semaphorin, cell adhesion molecule, or a combination of two or more of the foregoing.

14. The method of claim [[16]] 13, wherein the first molecular growth cue or the second molecular growth cue comprises Semaphorin 3A.

15. The method of claim 4, wherein:
the first molecular growth cue comprises GDNF; and
the second molecular growth cue comprises BDNF or PTN and Semaphorin 3A.

16. The method of claim 1 further comprising exposing the nerves of the first nerve type and/or the nerves of the second nerve type to a third molecular growth cue.

17. The method of claim 16, wherein the third molecular growth cue:
promotes myelination of the nerves of the first nerve type and/or the nerves of the second nerve type;
stimulates Schwann cells; or
a combination of both.

18. (canceled)

19. The method of claim 16, wherein the third molecular growth cue is provided in microparticles having an average diameter between 1500 and 3500 μM.

20. (canceled)

21. The method of claim 16, wherein the step of exposing the nerves of the first nerve type and/or the nerves of the second nerve type to a third molecular growth cue comprises using a sustained release profile for the third molecular growth cue for at least 20 days.

22. The method of claim 16, wherein the third molecular growth cue comprises a neuregulin (NRG), neuregulin 1 type III (NRG1-III), or both.

23. (canceled)

24. A device for promoting asymmetric nerve growth, the device comprising:
a lumen having a proximal end and a distal end; and
a matrix material disposed in the lumen;
wherein the proximal end of the lumen comprises a proximal opening operable to receive nerve tissue;
wherein the distal end of the lumen comprises a distal opening operable to receive nerve tissue;
wherein the matrix material defines one or more first microchannels and one or more second microchannels extending from the proximal end of the lumen toward the distal end of the lumen, the first microchannels differing from the second microchannels;
wherein a first molecular growth cue is disposed within the first microchannels;
wherein a second molecular growth cue is disposed within the second microchannels, the first molecular growth cue differing from the second molecular growth;
wherein the first molecular growth cue preferentially promotes growth of a first nerve type, as compared to a second nerve type differing from the first nerve type; and
wherein the second molecular growth cue preferentially promotes growth of the second nerve type, as compared to the first nerve type.

25-34. (canceled)
